SEQUENCE LISTING (194 sequences)

SEQ ID NO: 1 - DNA, 666 bp, Homo sapiens
  CDS (1)..(663)
atgagaaggtgtagaataagtgggaggcccccggcgccccccggtgtccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagggctgctactccccaccaccgtccccagccccgttctgttccgggctgggactctgcccccggagcaccctccccagctgacatcacccaatcccactccagccccaggcccctctgcccacgctgcacccagcaccaccagtgccctgacccccggacctgccgctgccgctgtcgacgccgcagcttcctccgttgtcaagggcggggcttagagctcaacccagacacctgcaggtgccggaagctgcgaaggtga

SEQ ID NO: 2 - protein, 221 aa, Homo sapiens
MRRCRISGRPPAPPGVPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITQSHSSPRPLCPRCTQHHQCPDPRTCRCRCRRRSFLRCQGRGLELNPDTCRCRKLRR

SEQ ID NO: 3 - protein, 215 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 4 - protein, 14 aa, Homo sapiens
  UNSURE (2): May be any amino acid
PXCVXXXRCXGCCN

SEQ ID NO: 5 - DNA, 29 bp, Artificial Sequence
  Description of Artificial Sequence DNA Primer
gactgcatgcaccagaggaaagtggtgtc

SEQ ID NO: 6 - DNA, 29 bp, Artificial Sequence
  Description of Artificial Sequence DNA Primer
gactagatctccttcgcagcttccggcac

SEQ ID NO: 7 - DNA, 29 bp, Artificial Sequence
  Description of Artificial Sequence DNA Primer
gcatggatcccagcctgatgcccctggcc

SEQ ID NO: 8 - DNA, 30 bp, Artificial Sequence
  Description of Artificial Sequence DNA Primer
gcattctagaccctgctgagtctgaaaagc

SEQ ID NO: 9 - DNA, 733 bp, Homo sapiens
gggatccggagcccaaatcttctgacaaaactcacacatgcccaccgtgcccagcacctgaattcgagggtgcaccgtcagtcttcctcttccccccaaaacccaaggacaccctcatgatctcccggactcctgaggtcacatgcgtggtggtggacgtaagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaacccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccgcgactctagaggat

SEQ ID NO: 10 - protein, 5 aa, Homo sapiens
  UNSURE (3): May be any amino acid
WSXWS

SEQ ID NO: 11 - DNA, 86 bp, Artificial Sequence
  Description of Artificial Sequence DNA Primer
gcgcctcgagatttccccgaaatctagatttccccgaaatgatttccccgaaatgatttccccgaaatatctgccatctcaattag

SEQ ID NO: 12 - DNA, 27 bp, Artificial Sequence
  Description of Artificial Sequence DNA Primer
gcggcaagctttttgcaaagcctaggc

SEQ ID NO: 13 - DNA, 271 bp, Artificial Sequence
  Description of Artificial Sequence PCR fragment
ctcgagatttccccgaaatctagatttccccgaaatgatttccccgaaatgatttccccgaaatatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 14 - DNA, 32 bp, Homo sapiens
gcgctcgagggatgacagcgatagaaccccgg

SEQ ID NO: 15 - DNA, 31 bp, Homo sapiens
gcgaagcttcgcgactccccggatccgcctc

SEQ ID NO: 16 - DNA, 12 bp, Artificial Sequence
  Description of Artificial Sequence DNA Primer
ggggactttccc

SEQ ID NO: 17 - DNA, 73 bp, Artificial Sequence
  Description of Artificial Sequence DNA Primer
gcggcctcgaggggactttcccggggactttccggggactttccgggactttccatcctgccatctcaattag

SEQ ID NO: 18 - DNA, 256 bp, Artificial Sequence
  Description of Artificial Sequence PCR Fragment
ctcgaggggactttcccggggactttccggggactttccgggactttccatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 19 - DNA, 663 bp, Homo sapiens
  CDS (1)..(618)
atgagaaggtgtagaataagtgggaggcccccggcgccccccggtgtccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagggctgctactccccaccaccgtccccagccccgttctgttccgggctgggactctgcccccggagcaccctccccagctgacatcacccatcccactccagccccaggcccctctgcccacgctgcacccagcaccaccagtgccctgacccccggacctgccgctgccgctgtcgacgccgcagcttcctccgttgtcaagggcggggcttagagctcaacccagacacctgcaggtgccggaagctgcgaaggt

SEQ ID NO: 20 - protein, 206 aa, Homo sapiens
MRRCRISGRPPAPPGVPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAVDAAASSVVKGGA

SEQ ID NO: 21 - DNA, 531 bp, Homo sapiens
aattcggaacgatgaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgcctgacgatggcctggagtgtgtgcccactggcagcaccaagtccggatgcagatcctcatgatccggtacccgagcagtcagctgggggagatgtccctggaagaacacagccagtgtgaatgcagacctaaaaaaaaggacagtgctgtgaaccagacagcccaggccctctgcccacgctgcacccagcaccaccagcgccctgacccccggacctgccgctgccgctgccgacgccgcagcttcctccgttgccaagggcggggcttagagctcaacccagacacctgcaggtgccggaagctgcgaaggtgacacatggcttttcagactcagcagggtgacttgcctcagaggctatatcccagt

SEQ ID NO: 22 - DNA, 445 bp, Homo sapiens
aacagctggtgcccagctgcgtgactgtgcacgctgtggtggctgctgcctgacgatggcctggagtgtgtgcccactgcagcaccaagtccggatgcagatcctcatgatccggtacccgagcagtcagctgggggagatgtccctggaagaacacagccagtgtgaatgcagacctaaaaaaaaggacagtgctgtgaaccagacagggctgccactccccaccaccgtccccagccccgttctgttccgggctgggactctgcccccggagcaccctccccagctgacatcacccatcccactccagccccaggcccctctgcccacgctgcacccagcaccaccagcgccctgacccccggacctgccgctgccgctgccgacgccgcagcttcctccgttgccaagggcggggcttagagctcaacccagacacctgcag

SEQ ID NO: 23 - DNA, 415 bp, Homo sapiens
  unsure (85): May be any nucleic acid
cctggagtgtgtgcccactgggcagcaccaagtccggatgcagatcctcatgatccggtacccgagcagtcagctgggggagatntccctggaagaacacagccagtgtgaatgcagacctaaaaaaaaaaggacagtgctgtnaagccagacagggctgccactccccaccaccgtccccagccccgttctgttccgggctgggactctgcccccggagaaccctncccagctgacatcacccatcccactccagccccaggnccctctgcccacgntgcacccagcaccaccagngncctgacccccggacctnncgctgccgctgccgacgncgcagcttcctccgttgccaagggcggggcttagagctnaacccagacacctgnaggtgccggaagctgcgaaggtgaca

SEQ ID NO: 24 - DNA, 423 bp, Mus musculus
gcacgctgtggtggctgctgccctgacgatggcctggaatgtgtgcccactgggaacaccaagtccgaatgcagatcctcatgatccagtacccgagcagtcagctgggggagatgtccctggaagaacacagccaatgtgaatgcagaccaaaaaaaaaggagagtgctgtgaagccagacagccccaggatcctctgcccgccttgcacccagcgccgtcaacgccctgacccccggacctgccgctgccgctgcagacgccgccgcttcctccattgccaagggcggggcttagagctcaacccagacacctgtaggtgccggaagccgcgaaagtgacaagctgctttccagactccatgggcccggctgcttttatggccctgcttcacagggagaagagtggagcacaggcgaacct

SEQ ID NO: 25 - DNA, 341 bp, Homo sapiens
  unsure (129): May be any nucleic acid
ccactccccaccaccggtccccagccccgttctgttccgggctgggactctgcccccggagcaccctccccagctgacatcacccatcccactccagccccagggccctctgcccacgctgcacccagnancaccagcgccctgacccccggacctgccgctgccgctgccgacgccgcagcttcctccgttgccaagggggggcttagagctcaacccagacacctgcaggtgccngaagctgcgaaggtgacanatggcttttcagactcagcagggtgacttgcctnagaggctatatcccagtgggggaacaaagaggagcctngtaaaaagcagcc

SEQ ID NO: 26 - DNA, 312 bp, Homo sapiens
  unsure (160): May be any nucleic acid
gtccccagccccgttctgttccgggctgggactctgcccccggagcaccctccccagctgacatcacccatcccactccagccccaggcccctctgcccacgctgcacccagcaccaccagcgccctgacccccggacctgccgctgccgctgccgacgncgcagcttcctccgttgccaagggggggcttagagctcaacccagacacctgcaggtgccggaagctgcgaaggtgacacatgggttttcagactcagcagggtgacttgcctnagaggntatatcccagttgggggaacaaagagggagcc

SEQ ID NO: 27 - DNA, 553 bp, Homo sapiens
  unsure (100)..(101): May be any nucleic acid
acccatcccactccagccccaggcccctctgcccacgctgcacccagcaccaccagcgccctgacccccggacctgccgctgccgctgccgacgccgcannttcctccgttgccaaggnggggcttagagctcaacccagacacctgcaggtgccggaagctgcgaaggtgacacatggcttttcagactcagcagggtgacttgcctcagaggctatatcccagtgggggaacaaagaggagcctggtaaaaaacagccaagcccccaagacctcagcccaggcagaagtgctctaggacctgggcctctcagagggctcttctgccatcccttgtctccctgaggccatcatcaaacaggacagagttggaagaggagactgggangcagcaagaggggtccataccagctcaggggagaatggagtactgtctcagtttctaancactctgtgcaagtaagcatcttacaactggctcttcttcccctcactaagaaganccaaactttgataatgggattgggtttgtacaagaatgtgancccaancc

SEQ ID NO: 28 - DNA, 231 bp, Mus musculus
aaaaggagagtgctgtgaagcagacagcccaggatcctctgcccgccttgcacccagcgccgtcaacgccctgacccccggacctgccgctgccgctgcagacgccgccgcttcctccattgccaagggcggggcttagagctcaacccagacacctgtaggtgccggaagccgcgaaagtgacaagctgctttccagactccacgggcccggctgcttttatggccctgc

SEQ ID NO: 29 - DNA, 523 bp, Homo sapiens
  unsure (8): May be any nucleic acid
ctctgccnacgctgcacccagcaccaccagcgccctgacccccggacctgccgctgccgctgccgacgccgcagcttcctccgttgccaaggnggggcttagagctcaacccagacacctgcaggtgccggaagctgcgaaggtgacacatggcttttcagactcagcagggtgacttgcctcagaggctatatcccagtgggggaacaaagaggagcctggtaaaaaacagccaagcccccaagacctcagcccaggcagaaagctgctctaggacctgggcctctcagagggctcttctgccatcccttgtctccctgaggccatcatcaaacaggacagagttggaagaggagactnggaggcagcaagaggggtnacataccagctcaggggagaattggagtactgtctcagttttctaaccacttttgtgccaagtaaagcatcttacaacttggctcttcntccccttgacttnagaagacccaaanctctngcataaatggggattttgggcntt

SEQ ID NO: 30 - DNA, 225 bp, Homo sapiens
  unsure (64): May be any nucleic acid
cccacgctgcacccagcaccaccagcgccctgacccccggacctgccgctgccgctgccgacgncgcagcttcctccgttgccaagggcggggcttagagctcaacccagacacctgcagctgctggcacctggagctgtataagcaagtccaacattctaactcaggagtcagatacaggagggacaatgccagcagaggatgtcaagggctgtggtgaggggt

SEQ ID NO: 31 - DNA, 118 bp, Homo sapiens
ccagcgccctgacccccggacctgccgctgccgctgccgacgccgcagcttcctccgttgccaagggcggggcttagagctcaacccagacacctgcaggtgccggaagctgcgaagg

SEQ ID NO: 32 - DNA, 565 bp, Homo sapiens
tttttttttccttccatctcttttatcagggttgggggtcacagttcttgtaccaaagcccaaatcccattatgcagaggtttgggtcttcttagtgaggggaggaagagccagttgtaagatgcttacttgcacagagtggttagaaactgagacagtactccattctcccctgagctggtatgtgacccctcttgctgcctcccagtctcctcttccaactctgtcctgtttgatgatggcctcagggagacaagggatggcagaagagccctctgagaggcccaggtcctagagcagcttctgcctgggctgaggtcttgggggcttggctgttttttaccaggctcctctttgttcccccactgggatatagcctctgaggcaagtcaccctgctgagtctgaaaagccatgtgtcaccttcgcagcttccggcacctgcaggtgtctgggttgagctctaagccctgcccttggcaacggaggaaactgcggcgtcggcagcggcagtacagagtccgggggtcagggcgcttgtggtgctgggtgcagcgtgagggcaa

SEQ ID NO: 33 - DNA, 374 bp, Homo sapiens
  unsure (88): May be any nucleic acid
cccacgctgcacccagcaccaccagcgccctgacccccggacctgccgctgccgctgccgacgccgcagcttcctccgttgccaaggnggggcttagagctcaacccagacacctgcaggtccggaagctgcgaaggtgacacatggcttttcagactcagcagggtgacttgcctcagaggctatatcccagtggggaacaaagaggagcctggtaaaaaacagccaagcccccaagacctcagcccaggcagaagctgctctaggacctgggcctctcagagggctcttctgccatcccttgtctccctgagggcatcatcaaacaggacagagttggaagatgagactggggaggcagcaagaggggtcac

SEQ ID NO: 34 - DNA, 502 bp, Homo sapiens
ttttttccttccatctcttttatcggggttgggggtcacagttcttgtaccaaagcccaaatcccattatgcagaggtttgggtcttcttagtgaggggaggaagagccagttgtaagatgcttacttgcacagagtggttagaaactgagacagtactccattctcccctgagctggtatgtgacccctcttgctgcctcccagtctcctcttccaactctgtcctgtttgatgatggcctcagggagacaagggatggcagaagagccctctgagaggcccaggtcctagagcagcttctgcctgggctgaggtcttgggggcttggctgttttttaccaggctcctctttgttcccccactgggatatagcctctgaggcaagtcaccctgctgagtctgaaaagccatgtgtcaccttcgcagcttccggcacctgcaggtgtctgggttgagctctaagccccgcccttggcaacggaggaagctgcggcgtcagca

SEQ ID NO: 35 - DNA, 493 bp, Homo sapiens
ttttttccttccatctcttttatcagggttgggggtcacagttcttgtaccaaagcccaaatcccattatgcagaggtttgggtcttcttagtgaggggaggaagagccagttgtaagatgcttacttgcacagagtggttagaaactgagacagtactccattctcccctgagctggtatgtgacccctcttgctgcctcccagtctcctcttccaactctgtcctgtttgatgatggcctcagggagacaagggatggcagaagagccctctgagaggcccaggtcctagagcagcttctgcctgggctgaggtcttgggggcttggctgttttttaccaggctcctctttgttcccccactgggatatagcctctgaggcaagtcaccctgctgagtctgaaaagccatgtgtaaccttcgcagcttccggcacctgcaggtgtctgggttgagctctaagccccgcccttggcaacggaggaagctgcg

SEQ ID NO: 36 - DNA, 381 bp, Mus musculus
cgccgcttcctccattgccaagggcggggcttagagctcaacccagacacctgtaggtgccggaagccgcgaaagtgacaagctgctttccagactccacgggcccggctgcttttatggccctgcttcacagggagaagagtggagcacaggcgaacctcctcagtctgggaggtcactgccccaggacctggaccttttagagagctctctcgccatcttttatctcccagagctgccatctaacaattgtcaaggaacctcatgtctcacctcaggggccagggtactctctcacttaaccaccctggtcaagtgagcatcttctggctggctgtctcccctcactatgaaaaccccaaacttctaccaataacggga

SEQ ID NO: 37 - DNA, 490 bp, Homo sapiens
gtttctccttccatctcttttatcagggttgggggtcacagttcttgtaccaaagcccaaatcccattatgcagaggtttgggtcttcttagtgaggggaggaagagccagttgtaagatgcttacttgcacagagtggttagaaactgagacagtactccattctcccctgagctggtatgtgacccctcttgctgcctcccagtctcctcttccaactctgtcctgtttgatgatggcctcagggagacaagggatggcagaagagccctctgagaggcccaggtcctagagcagcttctgcctgggctgaggtcttgggggcttggctgttttttaccaggctcctctttgttcccccactgggatatagcctctgaggcaagtcacccctgctgagtcgaaaagccatgtgtcaccttcgcaggtttcggcacctgcaggtgtctgggttgagctctaagccccggcctttgcaacggaagaagct

SEQ ID NO: 38 - DNA, 297 bp, Homo sapiens
  unsure (56): May be any nucleic acid
cagcttcctccgttgccaagggggggcttagagctcaacccagacacctgcaggtnccggaagctgcgaaggtgacacatggcttttcagactcagcagggtgacttgcctcagaggctatatcccagtgggggaacaaagaggagcctggnaaaaaacagccaagcccccaagacctnagcccagggagaagctgctctaggacctgggcctntcagagggctcttcttgccatnccttgtcttcctgagggcatcattaaacaggacagagtttggagaggagattnggaggcaa

SEQ ID NO: 39 - DNA, 469 bp, Homo sapiens
tttttttccttccatctcttttatcagggttgggggtcacagttcttgtaccaaagcccaaatcccattatgcagaggtttgggtcttcttagtgaggggaggaagagccagttgtaagatgcttacttgcacagagtggttagaaactgagacagtactccattctcccctgagctggtatgtgacccctcttgctgcctcccagtctcctcttccaactctgtcctgtttgatgatggcctcagggagacaagggatggcagaagagccctctgagaggcccaggtcctagagcagcttctgcctgggctgaggtcttgggggcttggctgttttttaccaggctcctctttgttcccccactgggatatagcctctgaggcaagtcaccctgctgagtctgaaaagccatgtgtcaccttcgcagcttccggcacctgcaggtgtctgggttgagctctaagcccc

SEQ ID NO: 40 - DNA, 210 bp, Mus musculus
cccgagcagtcagctgggggagatgtccctggaagaacacagccaagtgtgaatgcaggttgcaaccgaggccaggtcacaaagtgtagcatggggtgggggtacaggcatgggcttgtaccttacttagtagggaccaggtgactgagagtagagccagaagtagtaggcatggatcctgggacaagagtgcaggaggcttgggtttct

SEQ ID NO: 41 - DNA, 289 bp, Homo sapiens
tagagctcaacccagacacctgcaggtgccggaagctgcgaaggtgacacatggcttttcagactcagcagggtgacttgcctcagaggctatatcccagtggggacacacagaggagcctggtaaaaaacagccaagcccccaagacctcagcccaggcagaagcttgctctaggacctcggcctctcagagggctcttctgccatcccctgtctccctgaggccatcatcaacaggacagagttggagaggagactcggaggcagcagagggtcacataccactcag

SEQ ID NO: 42 - DNA, 281 bp, Homo sapiens
ctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccgggcccccccggtgtccccgcgaggccccggggcggggtccgcggcccctgcgcgcccgggtgaaaataccactactctgatcgttttttcactgacc

SEQ ID NO: 43 - DNA, 284 bp, Homo sapiens
ctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggtcccccccggtgtccccgcgatgcccggggcggggtccctggccctgcgggctcggtgaaataccactactctgatcgttttttcactgacccggtgag

SEQ ID NO: 44 - DNA, 301 bp, Homo sapiens
ctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccgggcccccccggtgtccccgcgaggcctcggggcggggtccgcggcctctgcgcggccaggtgaaataccactactctgatcgttttttcactgacccggtgaggcgggggggcgagc

SEQ ID NO: 45 - DNA, 326 bp, Homo sapiens
cttaaggtagccaaatgcctcgtcatctaattattgacgcgcatgaatggatgaacgagattcccactgtccctacctactatccagcgaaaccacagccaagggaacgggcttggcggaatcagccgggaaagaagaccctgttgagcttgactctagtctggcacggtgaagagacatgagaggtgtagaataagtgggaggcccccgggcccccccggtgtccccgcgagtcccggggcggggtccgcggcctctgcggcacggttgtaaataccactactctgatcgttttttcactgacccggtgaggcgggggggcgagc

SEQ ID NO: 46 - DNA, 285 bp, Homo sapiens
  unsure (202): May be any nucleic acid
tttttgctgcatgattttattactataaatatacagtaaaaacgaaccaacgatgagcccatctgaacacatcagacggcagaacatgggagtcccagcggaccactctgcggcacgaacttcacgcaaagctctggcaccaggactgatggccagagcgtggggccttggtggtatttcaccggcggcccgcaggaacacnccgccaccaggcacccatcgcggggacaccgggggggcgccggggcctcccacttattctacacctctcatgtctcttcaccg

SEQ ID NO: 47 - DNA, 82 bp, Mus musculus
catgagaggtgtagaataagtgggaggcccccggcgcccggccccgtcctcgcgtcggggtcggggcacgccggcctcgcgg

SEQ ID NO: 48 - DNA, 320 bp, Homo sapiens
caaatgcctcgtcatctaattagtgacgcgcatgaatggatgaacgagattcccactgtccctaccatacaaaccagcgaaaccacagcacaagggaacgggcttggcggaatcagcggggaaagaagaccctgttgagcttgactctagtctggcacggtgaagagacatgagaggtgtagaataagtgggaggcccccggcgcccccccggtgtccccgcgaggcaccggggcggggtccgcggcctctgcgcgcactaggtgaaataccactactctgatcgttttttcactgacccggtgaggcgggggggcgagc

SEQ ID NO: 49 - DNA, 38 bp, Homo sapiens
  unsure (11): May be any nucleic acid
ctgctgccctnacgatggcctggagtgtgtgcccactg

SEQ ID NO: 50 - DNA, 499 bp, Rattus sp.
  unsure (47): May be any nucleic acid
gcagcgaaaccacagccaagggaacgggcttggcggaatcagcgggngaaagaagaccctgttgagcttgactctagtctggcacggtgaagagacatgagaggtgtagaataagtgggaggcccccggcgcccccccgttccccgcgaggggtcggggcggggtccgccggcctcgcgggccgccggtgaaataccactactctcatcgttttttcactgacccggtgaggcgggggggcgagccccgaggggctctcgcttctggcgccaagcgcccgtccgcgcgcgcgggcgggcgcgacccgctccggggacagtgccaggtggggagtttgactggggcggtacacctgtcaaacggtaacgcatgtgtccacaccaagacctgcangccgggagtcaggattcctccttccctgaggcactgaacacccgcggcacctccccacagcatgtctcaccacaatcctgttgctacatcagagtgtatttttgta

SEQ ID NO: 51 - DNA, 301 bp, Homo sapiens
  unsure (242): May be any nucleic acid
ctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccgggtcccccccggtgtccccgcgaggcccgggtcggggttccgcggccctgcggctcgngatgaaataccactactctgatcgttttttcactgacccggtgaggcgggggggcgagc

SEQ ID NO: 52 - DNA, 611 bp, Rattus sp.
  unsure (551): May be any nucleic acid
tcgtcctcccccttccccctccgcggggtcgggggttcccggggttcggggttctcctccgcgtcggcggttcccccgccgggtgcgccccccgggccgcggtttcccgcgcggcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccccccgttccccgcgaagggtcggngcggggtccgccggcctcgcgggccgccggtgaaataacactactctcatcgttttttca

SEQ ID NO: 53 - DNA, 655 bp, Rattus sp.
  unsure (597): May be any nucleic acid
tcccggggagcccggcgggtcgccggcgcggggttttcctccggcctcgtcctcccccttccccctccgcggggtcgggggttcccggggttcggggttctcctccgcgtcggcggttcccccgccgggtgcgccccccgggccgcggtttcccgcgcggcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccccccgttccccgcgaggggtcggngcggggtccgccggcctcgcgggccgccggtgaaataacactactctcatcgtttttt

SEQ ID NO: 54 - DNA, 655 bp, Rattus sp.
tcccggggagcccggcgggtcgccggcgcggggttttcctccggcctcgtcctcccccttccccctccgcggggtcgggggttcccggggttcggggttctcctccgcgtcggcggttcccccgccgggtgcgccccccgggccgcggtttcccgcgcggcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccccccgttccccgcgaagggtcggggcggggtccgccggcctcgcgggccgccgggtgaaataccactactctcatcgttttt

SEQ ID NO: 55 - DNA, 661 bp, Rattus sp.
tcccggggagcccggcgggtcgccggcgcggggttttcctccggcctcgtcctcccccttccccctccgcggggtcgggggttcccggggttcggggttctcctccgcgtcggcggttcccccgccgggtgcgccccccgggccgcggtttcccgcgcggcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccccccgttccccgcgaggggtcggggccgggtccgccgggctcgcgggccgccggtgaaataccactactctcatcgttttttcactga

SEQ ID NO: 56 - DNA, 251 bp, Homo sapiens
ctttttttttttctcttcttttttcttcttttttttttttttttctcaccgggtcagtgaaaaaacgatcagagtagtggtatttcaccggcggcccgcacggggctcccagccccgggcccctcgcggggacaccgggggggcgccgggggcctcccacttattctacacctctcatgtctcttcaccgtgccagactagagtcaagctcaacagggtcttctttccccgctgattccgccaagcccgtt

SEQ ID NO: 57 - DNA, 426 bp, Homo sapiens
ctaattagtgacgcgcatgaatggatgaacgagattcccactgtccctacctactatccagcgaaaccacagccaagggaacgggcttggcggaatcagccgggaaagaagaccctgttgagcttgactctagtctggcacggtgaagagacatgagaggtgtagaataagtgggaggcccccggcgcccccccggtgtccccgcgatggcccggggcggggtcacgcggccgtgcggccacggtgaaataccactactctgatcgttttttcactgacccggtgaggcgggggggcgagccccgaggggtctacgcttctggcgccaagccgcggaccgcgcgccgagcacacagtcttcccggggggaacaagtttgccaggtgggggagtttgactgggggcggtacacctgtcaaacggtaa

SEQ ID NO: 58 - DNA, 115 bp, Homo sapiens
cggggaaagaagaccctgttgagcttgactctagtctggcacggtgaagagacatgagaggtgtagaataagtgggaggcccccggcgccccccccggtgtccccgtgtcgacgc

SEQ ID NO: 59 - DNA, 540 bp, Homo sapiens
  unsure (12): May be any nucleic acid
ctggagtgtgtncccactgaggagtccaacatcaccatgcagattatgcggntcaaacctcaccaaggccagcacataggagagatgagcttcctacagcacaacaaatgtgaatgcagaccaaagaaagatagagcaagacaagaaaaatgtgacaagccgaggcggtgagccgtgcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatat

SEQ ID NO: 60 - DNA, 215 bp, Homo sapiens
ccggtcgacgccggggacaccgggggggcgccgggggctcccacttattctacacctctcatgtctcttcaccgtgccagactagagtcaagctcaacagggtcttctttccccgctgattccgccaagcccgttcccttggctgtggtttcgctggatagtaggtagggacagtgggaatctcgttcatccattcatgcgcgtcactaattaga

SEQ ID NO: 61 - DNA, 250 bp, Homo sapiens
ttgcagggccggcggacccgcccgggcccctcgcgggacaccgggggggcgccggggcctcccacttattctacacctctcatgtctcttcaccgtgccagactagagtcaagctcaacagggtcttctttccccgctgattccgccaagcccgttcccttggctgtggtttcgctggatagtaggtagggacagtgggaatctcgttcatccattcatgcgcgtcactaattagatgacgaggcatttg

SEQ ID NO: 62 - DNA, 303 bp, Homo sapiens
ctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccgggccccccccggtgtccccgcgaggcccaggggcggggtccgcggccactgcaacgcctggtcgaaataccactactctgatcgttttttcactgacccggtgaggcgggggggcgagc

SEQ ID NO: 63 - DNA, 201 bp, Homo sapiens
ctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcccgccccggggtcga

SEQ ID NO: 64 - DNA, 224 bp, Homo sapiens
gtcgacaattcaatgaagcgcggggacaccggggggacgcctggggctcccacttattctacacctctcatgtctcttcaccgtgccagactagagtcaagctcaacagggtcttctttccccgctgattccgccaagcccgttcccttggctgtggtttcgctggatagtaggtagggacagggggaatctcgttcatccattcatgcgcgtcactaattaga

SEQ ID NO: 65 - DNA, 283 bp, Homo sapiens
gatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccccccggtgtccccgctgaggcccggggcggggtccgtggccctgcggccctggtgaaataccactactctgatcgttttttcactgacccggtgaggcgggggggcgagctcgagg

SEQ ID NO: 66 - DNA, 321 bp, Homo sapiens
  unsure (97)..(98): May be any nucleic acid
gccagaagcgagacccctcgggcgctccccccgcctcaccgggtcagtgaaaaaacgatcagagtagtggtatttcaccggcggcccgcacgcagcnnacccgccccgggcccctcgcgggtacaccgggggggcgccgggggcctcccacttattctacacctctcgtttctcttcagcgtgccagactagagtcaagctcaacagggtcttctttccccgctgattccgccaagcccgttcccttggctgtggtttcgctggatagtaggtagggacagtgggaatctcgttcatccattcatgcgcgtcactaattag

SEQ ID NO: 67 - DNA, 231 bp, Mus musculus
ctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccggccccgtcctcgcgtcggggtcggggcacgccggcctcgc

SEQ ID NO: 68 - DNA, 193 bp, Homo sapiens
ctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccccc

SEQ ID NO: 69 - DNA, 306 bp, Mus musculus
tcggatccaagcggaccttgcgccagaagcgagacccctcggcgcgcccccccgcctcaccgggtcagtgaaaaaacgatgagagtagtggtatttcaccggcggcccggagccgcgtgccccgaccccgactgcgaggacggggccgggcgccgggggcctcccacttattctacacctctcatgtctcttcaccgtgccagactagagtcaagctcaacagggtcttctttccccgcatcattctcgcaagcccgttcccttggctgtggtttcgctggatagtaggtagggacagtgggaatc

SEQ ID NO: 70 - DNA, 308 bp, Mus musculus
cccagtcaaactccccacctggcactgtccccggagcgggtcgcgcccgcccgcacgcgcggagcggacgcttggcgccagaagcgagagccctcggcgcgcccccccgcctcaccgggtcagtgaaaaaacgatgagagtagtggtatttcaccggcggcccgcgaggccggcgtgccccgaccccgacgcgaggacggggccgggcgccgggggcctcccacttattctacacctctcatgtctcttcaccgtgccagactagagtcaagctcaacagggtcttctttccccgctgattccgccaa

SEQ ID NO: 71 - DNA, 332 bp, Mus musculus
attgggatccaacgccagaagcgagacccctcggcgcgcccccccgcctcaccgggtcagtgaaaaaacgatgagagtagtggtatttcaccggcggcccgcgaggccggcgtgccccgaccccgacgcgaggacggggccgggcgccgggggcctcccacttattctacacctctcatgtctcttcaccgtgccagactagagtcaagctcaacagggtcttctttccccgctgattcatcttagcccgttcccttggctgtggtttcgctggatagtaggtagggacagtgggaatctcgttcatccattcatgcgcgtcactaattaga

SEQ ID NO: 72 - DNA, 340 bp, Mus musculus
ctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccggccccgtcctcgcgtcggggtcggggcacgccggcctcgcgggccgccggtgaaataccactactctcatcgttttttcactgacccggtgatgcgggggggcgtagccgagggctctcgcttctggcgccaacgtccgtcccgcgcgt

SEQ ID NO: 73 - DNA, 343 bp, Mus musculus
cgcccgcacgccgacaggacgcttggcgccagaagcgagacccctcgcgccccccccgcctcaccgggtcagtgaaaaaacgatgagagtagtggtatttcaccggcggcccggaggcacgctgcgcccgaccccgacgcgaggactgggccgggcgccgggggcctcccacttattctacacctctcatgtctcttcaccgtgccagactagagtcaagctcaacagggtcttctttccccgctgattccgccaagcccgttccattggctgtggtttcgctggatagtaggtagggacagtgggaatctcgttcatccattcatgcgcgtcactaattaga

SEQ ID NO: 74 - DNA, 424 bp, Mus musculus
attcggatccaacttcgtactgaaaatcaagatcaagcgagcttttgcccttctgctccacgggaggtttctgtcctccctgagctcgccttaggacacctgcgttaccgtttgacaggtgtaccgctcccagtcaaactccccacctggcactgtccccggagcgggtcgccccgccgcacgcgcgcacggacgcttgggccagaagcgagagccctcgggtgcccccccgcctcaccgggtcagtgaaaaaacgatgagagtagtggtatttcaccggcggcccgcgagccggcgtgccccgaccccgacgcgaggacggggccgggcgccgggggcctcccacttattctacacctctcatgtctcttcaccgtgccagactagagtcaagctcaacagggtcttctttccccgctgattc

SEQ ID NO: 75 - DNA, 487 bp, Mus musculus
gggaacgggcttggcggaatcagcggggaaagaagaccctgttgagcttgactatagtctggcacggtgaagagacatgagaggtgtagaataagtgggaggcccccggcgcccggccccgtcctcgcgtcggggtcggggcacgccggcctcgcgggccgccggtgaaataccactactctcatcgttttttcactgacccggtgaggcgggggggcgagcccggagggcgtctcgcttctggagcacgcgctattgctcacaggcgatggcttggctcacctgcaggctgtggaccccagcagctgctctgcatcatccctcatgtgcaggtgtttgccgtgtagcccctaagcagaaggaatttgtcatcactagcctgaaggagcgtggttatgtgacccttatgtgtggagatggcaccaacgatgttggcgcactgaagcacgctgaatgtggtgtagcgctcctggccaatggccctgag

SEQ ID NO: 76 - DNA, 508 bp, Mus musculus
aattcggatccatcgcggtgaccttgaagcctaggcgcgggccgggtggagccgccgcagtgcagatcttggtggtagtagcaaatattcaaacgagaactttgaaggccgaagtggagaagggttccatgtgaacagcagttgaacatgggtcagtcggtcctgagagatgggcgagtgccgttccgaaggacgggcgatggcctccgttgccctcggccgatcgaaagagagcccctcgggcgtcgcccccccagcctcaccgggtcagtgaaaaaacgatgagagtagtggtatttcaccggcggcccgcgaggccggcgtgccccgaccccgacgcgaggacggggccgggcgccgggggcctcccacttattctacacctctcatgtctcttcaccgtgccagactagagtcaagctcaacagggtcttctttccccgctgattccgccaagcccgttcccttggctgtggtttcgctggatagtaggtagggacagtgggaa

SEQ ID NO: 77 - DNA, 585 bp, Mus musculus
cttgactctagtctggcacggtgaagagacatgagaggtgtagaataagtgggaggcccccggcgcccggccccgtcctcgcgtcggggtcggggcacgccggcctcgcgggcgccggtgaaataccactactctcatcgttttttcactgacccggtgatgcgggggggcgagcccgaggggctctcgcttctggcgccaagcgtccgtcccgcgcgtgcgatgggcgcgaccctgtccggggacagtgccaggtgtggagtttgactggggcggtacacctgtcaaacggtaacgcaggtgtccttaggcgagctcagggaggacagaaacctcccgtggagctcaagagcaaccctaccttgaacgagtaccacaccagggccaaaacccacctgaagacacttggcgagaaagccagacctgcgctggaggacctgcgccatagtctgatgcccatgctggagacgcttaagacccaagtcgagagtgtgatcgacaaggccagcgagactctgactgcccagtgagttgccgctttcactcccacccgcgaattggtttcttcgataagctttcagaatg

SEQ ID NO: 78 - DNA, 149 bp, Homo sapiens
ccagcgaaaccacagccaagggaacgggcttggcggaatcagcggggaaagaagaccctgttgagcttgactctagtctggcacggtgaagagacatgaggggtgtagaataagtgggaggcccccggcgcccccccggtgtccccgcg

SEQ ID NO: 79 - DNA, 231 bp, Homo sapiens
gccacagccaagggaacgggcttggcggaatcagcggggaaagaagaccctgttgagcttgactctagtctggcacggtgaagagacatgagaggtgtagaataagtgggaggcccccgggccccccccggtgtccccgcgagtcccgggcggggtcccgacccctgcgcgccgggtgaaataccactactctgatcgttttttcactgacccggtgaggcgggggggcga

SEQ ID NO: 80 - DNA, 300 bp, Homo sapiens
ctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccgggacgcccccggtgtccccgcgaggcccggggcggggttccgcggccctgcggcccggtggaagataccactactctgatcgttttttcactgacccggtgaggcgggggggcgag

SEQ ID NO: 81 - DNA, 314 bp, Mus musculus
aagcgagacccctcggcgcgcccccccgcctcaccgggtcagtgaaagaacgatgagagtagtggtatttcaccggcggcccgcgaggccggcgtgccccgaccccgactcgaggacggggccgggcgccgggggcctcccacttattctacacctctcatgtctcttcaccgtgccagactagagtcaagctcaacagggtcttctttccccgctgattccgccaagcccgttcccttggctgtggtttcgctggatagtaggtagggacagtgggaatctcgttcatccattcatgcgcgtcactagttaga

SEQ ID NO: 82 - DNA, 171 bp, Homo sapiens
attagtgacgcgcatgaatggatgaacgagattcccactgtccctacctactatccagcgaaaccacagccaagggaacgggcttggcggaatcagcggggacaccggaggggcgccgggggcctcccacttattctacacctctcatgtctcttcgtcgacgcggccgca

SEQ ID NO: 83 - DNA, 412 bp, Mus musculus
ctcaacccagacacctgtaggtgccggaagccgcgaaagtgacaagctgctttccagactccacgggcccggctgcttttatggccctgcttcacagggagaagagtggagcacaggcgaacctcctcagtctgggaggtcactgccccaggacctggaccttttagagagctctctcgccatcttttatctcccagagctgccatctaacaattgtcaaggaacctcatgtctcacctcaggggccagggtactctctcacttaaccaccctggtcaagtgagcatcttctggctggctgtctcccctcactatgaaaaccccaaacttctaccaataacgggatttgggttctgttatgataactgtgacacacacacacactcacactctgataaaagagatggaagac

SEQ ID NO: 84 - DNA, 415 bp, Mus musculus
ctcaacccagacacctgtaggtgccggaagccgcgaaagtgacaagctgctttccagactccacgggcccggctgcttttatggccctgcttcacagggagaagagtggagcacaggcgaacctcctcagtctgggaggtcactgccccaggacctggaccttttagagagctctctcgccatcttttatctcccagagctgccatctaacaattgtcaaggaacctcatgtctcacctcaggggccagggtactctctcacttaaccaccctggtcaagtgagcatcttctggctggctgtctcccctcactatgaaaaccccaaacttctaccaataacgggatttgggttctgttatgataactgtgacacacacacacactcacactctgataaaagagatggaagacact

SEQ ID NO: 85 - DNA, 356 bp, Mus musculus
tcggcacagggcgccgcgcacgcgcgacggaccttggcgccagaagcgagacccctcgggcgcccccccgcctcaccgggtcagtgaaaaaaacgatgagagtagtggtatttcaccggcggcccgcgaggccgcgtgccccgaccccgacgcgaggatcgggccgggcgccgggggcctcccacttattctacacctctcatgtctcttcaccgtgccagactagagtcaagctcaacagggtcttctttccccgctgattccgccaagcccgttcccttggctgtggtttcgcaggttcgtaggtagggacagtgggaatctcgttcatccattcatgcgcgtcactaattaga

SEQ ID NO: 86 - DNA, 418 bp, Mus musculus
  unsure (86): May be any nucleic acid
aacgagggtttgacaggtgtaccgccccagtcaaactccccagctggcactgtccccggagcggtcgcgcccgccgcacgcgcgancggacgcttgggccagaagcgagacccactcggggcaccccccgcctcaccgggtcagtgaaaaaacgatgagagtagtggtatttcaccggcggcccgcgaggaggcagnccccttacccactgccgcgaggacggggccgggcgccaggggcctcccacttattctacacctctcatgtctcttcaccgtgccagactagagtcaagctcaacagggtcttctttccccgctgattccgccaagcccgttcccttggctgtggtttcgctggatagtaggtagggacagtgggaatctcgttcatccattcatgcgcgtcactaattaga

SEQ ID NO: 87 - DNA, 463 bp, Mus musculus
ttcggatcatctttctgtcctccctgagctcgccttaggacacctgcgttaccgtttgacaggtgtatctccccagtcaaactccccacctggcactgtccccggagcgggtcgccccgccgcacgcgcggacggacgcttggcgccagaagcgagagccctcgggtgttccccccgcctcaccgggtcagtgactcaacgatgagagtagtggtatttcaccggcggcccgcgagaccggcgtgccccgaccccgacgcgaggacggggccgggcgccgggggcctcacacttattctacacctctcatgtctcttcaccgtgccagactagagtcaagctcaacagggtcttctttccccgctgattccgccaagcccgttcccttggctgtggtttcgctggatagtaggtagggacagtgggaatctcgttcatccattcatgcgcgtcactaattaga

SEQ ID NO: 88 - DNA, 560 bp, Rattus sp.
gggggttcccggggttcggggttctcctccgcgtcggcggttcccccgccgggtgcgccccccgggccgcggtttcccgcgcggcgcctcgcctcggccggcgcctagcagccgacttagaactggtgcggaccacgggaatccgactgtgtaattaaaacaaagcatcgcgaaggcccgcggcgggtgttgacgcgatgtgatttctgcccagtgctctgaatgtcaaagtgaacaaattcaatgaagcgcgggtaaacggcgggagtaactatgactctcttaaggtagccaaatgcctcgtcagctaattactgactcgcatgaatggatgaacgagattccactgtccctacctactatccaccgaaatcacagccacaggaacgggcttggcagaatcagcggggaaagaacacctgttgagcttgactctactctgggcacgtgaagacacatcacaggtgtagaataagtgggaggcccccggcacaccccgtttcgcgcgaggggtcaggcggggtccgcgagatcgcgggccccgatgaaatacactatcc

SEQ ID NO: 89 - DNA, 294 bp, Mus musculus
aaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccggccccgggtcctccagcgttgaacacttccttgcttttttcacatgttttatggaattgttcacatgatttgaaataataaaatgtaga

SEQ ID NO: 90 - DNA, 308 bp, Homo sapiens
  unsure (63): May be any nucleic acid
ctaattagtgacgcgcatgaatggatgaacgagattcccactgtccctacctactatccagcngaaaccacagccaagggaacgggcttggcggaatcagcggggaaagaagaccctgttgagcttgactctagtcttggcacgggtgaagagacatgagaggtgtagaataagtgggaggcccccgnncccccccaggtgtccccgcggagccacagggccggggtccncggcccttgcgncgcnggttgaaataccactactctgatcgttttttcactgacccggtgatgngaggggacgagcca

SEQ ID NO: 91 - DNA, 591 bp, Rattus sp.
gaaaaagcttgcggccgatcccggggagcccggcgggtcgccggcgcggggttttcctccggcctcgtcctcccccttccccctccgcggggtcgggggttcccggggttcggggttctcctccgcgtcggcggttcccccgccgggtgcgccccccgggccgcggtttcccgcgcggcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtaaaataagtgggaggcccccggcgccc

SEQ ID NO: 92 - DNA, 264 bp, Homo sapiens
  unsure (6): May be any nucleic acid
tctccngatgatgatgcacagccttcancgggggacatttaagacgcagaacaccaggtccaggctgcagctgcgggactcagaggcgaagttgaggggctcaggaaggacgaagaaccacccttnagagaagaggcagcagcagcggcggcagcagcagcggcagganccccaccactgccacatttnccaggaaacaatgctgctagcgacattcaagctgtgcgctgggagctcctacagacacatgcgcaacatgaaggg

SEQ ID NO: 93 - DNA, 207 bp, Homo sapiens
ctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggaccccgccccgggcgtcgacgc

SEQ ID NO: 94 - DNA, 203 bp, Homo sapiens
ctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggccccggcccgccccgggttcgacgc

SEQ ID NO: 95 - DNA, 566 bp, Rattus sp.
gtagcccggcgggtcgccggcgcggggttttcctccggcctcgtcctcccccttccccctccgcggggtcgggggttcccggggttcggggttctcctccgcgtcggcggttcccccgccgggtgcgccccccgggccgcggtttcccgcgcggcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggggcc

SEQ ID NO: 96 - DNA, 572 bp, Rattus sp.
tcccggggagcccggcgggtcgccggcgcggggttttcctccggcctcgtcctcccccttccccctccgcggggtcgggggttcccggggttcggggttctcctccgcgtcggcggttcccccgccgggtgcgccccccgggccgcggtttcccgcgcggcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtacaataagtgggaggcccccggcgcc

SEQ ID NO: 97 - DNA, 627 bp, Rattus sp.
ccggcgcggggttttcctccggcctcgtcctcccccttccccctccgcggggtcaggggttcccggggttcggggttctcctccgcgtcggcggttcccccgccgggtgcgccccccgggccgcggtttcccgcgcggcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacagtgaagagacatgagaagtgtagaataactgggaggcccctggagccccaccgttccccgcgatgggtccgggcagggtccgccggcctcgcgggccgccggtgaaataccactactctcatcg

SEQ ID NO: 98 - DNA, 297 bp, Mus musculus
catctaattagtgacgcgcatgaatggatgaacgagattcccactgtccctacctactatccagcgaaaccacagccaagggaacgggcttggcggaatcagcggggaaagaagaccgtgttgagcttgactctagtctggcacggtgaagagacatgagaggtgtagaataagtgggaggccccgcgcccggccccgtcctcgcgtcggggtcggggcacgccggcctcgcggccgccggtgaaataccactactctcatcgttttttcactgacccggtgaggcgggggggcgag

SEQ ID NO: 99 - DNA, 206 bp, Homo sapiens
ctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggccccggaccccgccccgggctacgacgc

SEQ ID NO: 100 - DNA, 578 bp, Rattus sp.
gcggacgctcccgaggagcccggcgggtcgccggcgcggggttttcctccggcctcgtcctcccccttccccctccgcggggtcgggggttcccggggttcggggttctcctccgcgtcggcggttcccccgccgggtgcgccccccgggccgcggtttcccgcgcggcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcctcgtcatctaattagtgacgcgcatgaatggatgaacgagattcccactgtccctacctactatccagcgaaaccacagccaagggaacgggcttggcggaaacagcggggaaagaagacctgttgagcttgactctagtctggcacggtgaagagacatgagatgtgtagaataagtgggaggcccccggcgc

SEQ ID NO: 101 - DNA, 215 bp, Homo sapiens
ctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggccccgggacccccacttattctaacctctcagtcgac

SEQ ID NO: 102 - DNA, 215 bp, Homo sapiens
gcctcgtcgacagaatattttgggggtcgccggggtcctctgacttattctacacctctcatgtttcttcatcgtgccagactagattcaagctcaacagggtcttctttccccgctgattccgccaagcccgttcccttggctgtggtttcgctggatagtaggtagggacagtgggaatctcgttcatccattcatgcgcgtcactaattaga

SEQ ID NO: 103 - DNA, 322 bp, Mus musculus
attagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggccccgcgcccggccccgtcctcgcgtcggggtcgggcacgccgcctcgcgggcgccggtgaaataccactactctcatcgttttttcactgacccggtgaggcgggggggcgagcccgaggcgtctcgcttctggcgccaacgtccg

SEQ ID NO: 104 - DNA, 391 bp, Mus musculus
attagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccgggcccggccccgtcctcgcgtcggggtcggggcacgccggcctcgcgggcgccgtgtgaaataccactactctcatcgttttttcactgacccggtgaggcgggggggcgagcctgaggggtctcgcttctggcgccaaggtccgtctcgcgcgtggctgggccgatcccgtctcggggacagtgccaggtgtggagtttgactggggcg

SEQ ID NO: 105 - DNA, 485 bp, Mus musculus
  unsure (459): May be any nucleic acid
cacagggagaggtgtagaataagtgggaggcccccgggcccggccccgtcctcgcgtcggggtcggggcacgccggcctcgcgggcgccgggtgaaataccactactctcatcgttttttcactgacccggtgatgcgggggggcgagctcgaggggtatctgcttctggcgccaacgtccgtcccgcgcgtgctgcgggcgcgaccccgtccggggacagtgccaggtgggactgtttgactggggcggtacacctgtcaaacggtaacgcaggtgtcctaaggcgagctcagggaggacagaaacctcccgtggagcagaagggcaaaagctcgcttgatcttgattttcagtacgaatacagaccgtgaaagccgggcctcacgatccttctgaccttttgggttttaagcaggaggtgtcagaaaagttaccccaggctgctttctgcttcacangtctctagccagtccctccacaaaca

SEQ ID NO: 106 - DNA, 255 bp, Homo sapiens
  unsure (4): May be any nucleic acid
gcgntcgacggcgctgcggcgacgcgtgaaataccactactctgatcgttttttcactgacccggtgaggcgggggcctcccacttattctacacctctcatgtctcttcaccgtgccagactagagtcaagctcaacagggtcttctttccccgctgattccgccaagcccgttcccttggctgtggtttcgctggatagtaggtagggacagtgggaatctcgttcatccattcatgcgcgtcactaattaga

SEQ ID NO: 107 - DNA, 163 bp, Homo sapiens
gatgaacgagattcccactgtccctatcctactatccagcgaaaccacagccaagggaacgggcttggcggaatcagcggggaaagaagaccctgttgagcttgactctagtctggcacggtgaagagacatgagaggtgtagaataagtgggaggcccccgg

SEQ ID NO: 108 - DNA, 207 bp, Homo sapiens
ccggccccttcagtccccagcccctgccccaactccgactcctgcacccagcccggcttcagccccgattccgactcccaccccggcaccagcccctgccccagctgcagccccagccggcagcacagggactggggggcccggggtaggaagtgggggggccgggagcgggggggatccggctcgacctggccttagccagcagca

SEQ ID NO: 109 - DNA, 506 bp, Mus musculus
caggggacagcccccactggctttgtctttggctcttctaccacctctgctccgtccaccggctccactgggttctcattcaccagtggcagtgcatcccagcctggagcctctggtgtcagccttggctctgtgggtagctctgcccagcccacagcactgtctggctctcccttcacaccagccactctggtgactactacagcaggagctacacagccagctgctgctgcacccactgctgccaccaccagtgcagggtctacactctttgcttccatagctgctgctcctgcctcatccagtgctacagggctctccctcccagctccggtgacaactgcagcaactcctagtgctgggactttgggcttcagctcaaggcccctggagcagctcctggtgcctccaccaccagcaccaccactaccactaccaccactactaccactgctgctgccgctgccgcctctaccacaaccactggctttgcttaagtctgaaac

SEQ ID NO: 110 - DNA, 938 bp, Mus musculus
  unsure (192): May be any nucleic acid
ggtgtagaataagtgggaggcccccgggcccggccccgtcctcgcgtcggggtcggggcacgccggcctcgcgggcgccggtgaaataccactactctcatcgttttttcactgacccggtgaggcgggggggcgagcccgagggctctcgcttctggcgccaacgtccggtccgcgcgtgcgcgcgccgancccccctcccggagaacagtgccaggtggggagtctgactggggcggtacacctgtcaaacggtaacgcaggtgtcctaaggcgagctcaaggaggacagaaacctcccgtggagcagaagggcaaaagctcgcttgatcttgattttcagtacgaatacagaccgtgaaagcggggcctcacgatccttctgaccttttgggttttaagcaggaggtgtcagaaaagttaccacagggataactggcttgtggcggccaagcgttcatagcgacgtcgctttttgactcttcgatgtcggctcttcctatcattgtgaagcagaattcaccaagcgttggattgttcagccactaactaggaacgtgaactgcgactaagacgtcgtgagaaggttagttttaccctactgatgatgtgttgttgccatggtaatctgctcagtaacagacgaacccagctagacactttgtgcaatggcttgctcaagagcaatgggcgactaccaacccttggaataagaatgaacgctctaaatcagaacccgccagggaaacgatacgcaagccacgagcttcggttggcccgaaaaacggtcccgtaagtcccgtcgggtaaacccgtactcacccggggggggataacccgcggggctcgaacgggttcgtccaaaacctccttttgaaacggctcgccaaaagcgcgctttgcagtcacttaacagtgcttgaactcggcaaaattca

SEQ ID NO: 111 - DNA, 239 bp, Homo sapiens
tcgacacggggacaccggggggggcgccgggggctcccacttattctacacctctcatgtctcttcaccgtgccagactagagtcaagctcaacagggtcttctttccccgctgattccgccaagcccgttcccttggctgtggtttcgctggatagtaggtagggacagtgggaatctcgttcatccattcatgcgcgtcactaattagatgacgaggcatttggtcgacgcggccgc

SEQ ID NO: 112 - DNA, 184 bp, Homo sapiens
ctaattagtgacgcgcatgaatggatgaacgagattcccactgtccctacctactatccagcgaaaccacagccaagggaacgggcttggcggaatcagcgaggaaagaagaccctgttgagcttgactctagtctggcacggtgaagagacatgagaggtgtagaataagtgggaggcccccg

SEQ ID NO: 113 - DNA, 184 bp, Homo sapiens
ctaattagtgacgcgcatgaatggatgaacgagattcccactgtccctacctactatccagcgaaaccacagccaagggaacgggcttggcggaatcagcctggaaagaagaccctgttgagcttgactctagtctggcacggtgaagagacatgagaggtgtagaataagtgggaggcccccg

SEQ ID NO: 114 - DNA, 240 bp, Mus musculus
ccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccgcgcccggccccgtcctcgcgtcggggtcggggcacgccgg

SEQ ID NO: 115 - DNA, 488 bp, Homo sapiens
  unsure (135): May be any nucleic acid
accatgcagattatgcggatcaaacctcaccaaggccagcacataggagagatgagcttcctacagcacaacaaatgtgaatgcagaccaaagaaagatagagcaagacaagaaaaatgtgacaagccgaggcgntgagcgngcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggaggcgagactccggcggaagatttcccgggcgggttgacccagcatggtccntttgggattgggtttcgccntttatttttntttgntgttaantcaccgaggcccggaagtttaggagattttattttttngggtttcncttaggacacanccacccac

SEQ ID NO: 116 - DNA, 498 bp, Homo sapiens
  unsure (134): May be any nucleic acid
ccatgcagattatgcggatcaaacctcaccaaggccagcacataggagagatgagcttcctacagcacaacaaatgtgaatgcagaccaaagaaagatagagcaagacaagaaaaatgtgacaagccgaggcgntgagcgngcaggaggaaggagcctccctcagggtttcgggaaccagatctctcaccaggaaagactgatacagaacgatcgatacagaaaccacgctgccgccaccacaccatcaccattcgacagaacagtccttaatccagaaacctgaaattgaaggaagagggagactcttgcgcagagcacttttggggtccggagggcggagattccggcgggaagattttcccgggcgggttgacccaggcntggtccnttttnggatttngggttttcgccnttttatttttttcttggnttgtttaaattcaccngaggcccggaagtttaggaganttttntttttttgggtttcccttagatt

SEQ ID NO: 117 - DNA, 348 bp, Homo sapiens
cagcacataggagagatgagcttcctacagcacaacaaatgtgaatgcagaccaaagaaagatagagcaagacaagaaaagtaagtggccctgactttagcacttctccctctccatggccggttgtcttggtttggggctcttggctacctctgttgggggctcccatagcctccctgggtcagggacttggtcttgtgggggacttgtggtggcagcaacaatgggatggagccaactccaggatgatggctctagggctagtgagaaaacatagccaggagcctggcacttcctttggaagggacaatgccttctgggtctccagatcattcctgaccaggactt

SEQ ID NO: 118 - DNA, 197 bp, Homo sapiens
cccccctcgaccggggggcctcccacttattctacacctctcatgtctcttcaccgtgccagactagagtcaagctcaacagggtcttctttccccgctgattccgccaagcccgttcccttggctgtggtttcgctggatagtaggtagggacagggggaatctcgttcatccattcatgcgcgtcactaattaga

SEQ ID NO: 119 - DNA, 154 bp, Homo sapiens
gacggggggcgccgggggctcccacttattctacacctctcatgtctcttcaccgtgccagactagagtcaagctcagcagggtcttctttccccgctgattccgccaagcccgttcccttggctgtggtttcgctggatagtaggtagggaca

SEQ ID NO: 120 - DNA, 263 bp, Homo sapiens
tttaacgtagactttcagctgcatgacctggtcccagccctgtagctcggaagccccaggaggtgaggttcacacccttagggctgggactctctgagccctaaggatgagattctaagttcagagatgagttgagggcttccctgaagggaccaaggaagtccctgtggcagaaacaataggagacaagaggtcagtggtgcggaggggaggggatggtacagaggggcctggggctggatcggagggttttggttgggagg

SEQ ID NO: 121 - DNA, 183 bp, Homo sapiens
ctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggccccc

SEQ ID NO: 122 - DNA, 304 bp, Homo sapiens
  unsure (5): May be any nucleic acid
ccggnagtgtatttaancggttctnttctgtcctctccaccacccccacccccctccctccggtgtgtgtgccgctgccgctgttgccgccgcagcctcgtcagcctgcgcagcccctcacaggaggcccagcccgagtgcagtccagaagcccccccagcggaggcggccagagtaaaagagcaagcttttgtgaagataatcgaagaacttttctcccccgtttgtttgttggagtggtgccaggtactggtntttggagaacttgtctacaaccagggattgattttaaagatgtcttttt

SEQ ID NO: 123 - DNA, 311 bp, Homo sapiens
ctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggccgccccctcgcccgtcacgcaccgcacgttcgtggggaacctggcgctaaaccattcgtagacgacctgcttctgggtcggggtttcgtacgtagcagagcagctccctcgctgcgatctattggtcgacgc

SEQ ID NO: 124 - DNA, 347 bp, Homo sapiens
  unsure (5): May be any nucleic acid
ccggnagtgtatttaatcggttctgtnctgtcctctccaccacccccacccccctccctccggtgtgtgtgccgctgccgctgttgccgccgcagcctcgtcagcctgcgcagcccctcacaggaggcccagcccgagtgcagtccagaagcccccccagcggaggcggcagagtaaaagagcaagcttttgtnagataatcgaagaacttttctcccccgtttgtttgttggagtggtgccaggtactggttttggagaacttgtctacaaccagggattgnttttaaaagatgtcttttttttaattttactttttttttaagcaccaaattttggttgtttttt

SEQ ID NO: 125 - DNA, 261 bp, Homo sapiens
  unsure (168): May be any nucleic acid
gacaggcagaggacaggtccagtctctgtggggactaacgaaagacatcatcatgatggaggacctgcctggtctagccccaggcccagcccccagcccagcccccagccccacagtagcccctgacccagccccagacgcctaccgtccagtggggctgaccaaggncgngctgtccctgcacacgtagaaggaagagcaagccttcctnagccgnttccgagacctgggcaggctgcgtggactngacagctcttncac

SEQ ID NO: 126 - DNA, 153 bp, Mus musculus
cagggaaagaagaccctgttgagcttgactctagtctggcacggtgaagagacatgagaggtgtagaataagtgggaggccccggcacccggccccgtcctcgcgtcggggtcggggcacgccggcctcgcgggcgcacggtgaaataccact

SEQ ID NO: 127 - DNA, 224 bp, Homo sapiens
ctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggccccgccgcgccccggggtcgacgcggccgcaatttagtagtagta

SEQ ID NO: 128 - DNA, 268 bp, Mus musculus
aacgatgagagtagtggtatttcaccgacggcccgcgaggccggcgtgcccccgacccccgacgcgaggcacggggccgggcgccggggcctcccacttattctacacctctcatgtctcttcaccgtgccagactagagtcaagctcaacagggtcttctttccccgctgattccgccaagcccgttcccttggctgtggtttcgctggatagtaggtagggacagtgggaatctcgttcatccattcatgcgcgtcactaattaga

SEQ ID NO: 129 - DNA, 138 bp, Rattus norvegicus
gcggccgcggcggcggcggcagcggcagcggcagctcagagcgcgcagcagcaacagcagcagcaggcgccgcagcagcaggcgccgcagctgagcccggtggcggacggccagccctcaaggggcggtcacaagtca

SEQ ID NO: 130 - DNA, 184 bp, Homo sapiens
ctaattagtgacgcgcatgaatggatgaacgagattccccctgtccctacctactatccagcgaaaccacagccaagggaacgggcttggcggaatcagcggggaaagaagaccctgttgagcttgactctagtctggcacggtgaagagacatgagaggtgtagaataagtgggacgcccccg

SEQ ID NO: 131 - DNA, 190 bp, Homo sapiens
ctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggccccggcgccgt

SEQ ID NO: 132 - DNA, 227 bp, Mus musculus
aaaaacgatgagagtagtggtatttcaccggcggcccgcgaggcagcgtgccccgaccccgacgcgaggacgggccgggcgccgggggctcccacttattctacacctctcatgtctcttcaccgtgcaagactagagtcaagctcaacagggtcttctttccccagctgattccgccaaggctcgttccattggctggtggttttcgctggatagtagggtaggga

SEQ ID NO: 133 - DNA, 182 bp, Homo sapiens
ctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccc

SEQ ID NO: 134 - DNA, 183 bp, Homo sapiens
  unsure (99): May be any nucleic acid
ctaattagtgacgcgcatgaatggatgaacgagattcccactgtccctacctactatccagcgaaaccacagccaagggaacgggcttggcggaatcancggggaaagaagaccctgttgagcttgactcgtagtctggcacggtgaagagacatganaggtgtagaataagtgggaggcccc

SEQ ID NO: 135 - DNA, 183 bp, Homo sapiens
ctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggccccg

SEQ ID NO: 136 - DNA, 191 bp, Homo sapiens
ctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccaggtcgacgc

SEQ ID NO: 137 - DNA, 294 bp, Rattus norvegicus
gcggccgcggcggcggcggcagcggcagcggcagctcagagcgcgcagcagcaacagcagcagcaggcgccgcagcagcaggcgccgcagctgagcccggtggcggacggccagccctcagggggcggtcacaagtcagcggccaagcaggtcaagcgccagcgctcgtcctctcccgaactgatgcgctgcaaacgccggctcaacttcagtggcttcggctacagccttccacagcagcagccggcagccgtggcgcgccgcaacgagcgcgagcgcaaccgggtcaagttg

SEQ ID NO: 138 - DNA, 362 bp, Rattus norvegicus
gcggccgcggcggcggcggcagcggcagcggcagctcagagcgcgcagcagcaacagcagcagcaggcgccgcagcagcaggcgccgcagctgagcccggtggcggacggccagccctcagggggcggtcacaagtcagcggccaagcaggtcaagcgccagcgctcgtcctctcccgaactgatgcgctgcaaacgccggctcaacttcagtggcttcggctacagccttccacagcagcagccggcagccgtggcgcgccgcaacgagcgcgagcgcaaccgggtcaagttggttaacctgggctttgccaccctccgggagcatgtccccaacggcgctgccaacaagaagatgagcaa

SEQ ID NO: 139 - DNA, 389 bp, Mus musculus
ctgctccgtcgcctgctgcttgttgcactgctgcagctggctcgcacccaggcccctgtgtcccagtttgatggccccagcaccagaagaaagtggtgccatggatagacgtttatgcacgtgccacatgccagcccagggaggtggtggtgcctctgagcatggaactcatgggcaatgtggtcaaacaactagtgcccagctgtgtgactgtgcagcgctgtggtggctgctgccctgacgatggcctggaatgtgtgcccactgcgaacaccaagtccgaatgcagatcctcatgatccagtacccgagcagtcagctgggggagatgtcctggaagaacacagccaatgtgaatgcagaccaaaaaaaaaggagagtgctgtgaa

SEQ ID NO: 140 - DNA, 491 bp, Mus musculus
  unsure (227): May be any nucleic acid
gagagtgctgtgaaccagacagggttgccataccccaccaccgtccccagccccgctctgttccgggctgggactctaccccgggagcatcctccccagctgacatcatccatcccactccagccccagggtcctctgcccgccttgcacccagcgccgtcaacgccctgacccccggacctgccgctgccgctgcagacgccgccgcttcctccattgccaaggcnggggcttagagctcaacccagacacctgtaggtgccggaagccgcgaaagtgacaagctgctttccagactccacgggcccggctgcttttatggccctgcttcacagggagaagagtggagcacaggcgaacctcctcagtctgggaggtcactgccccaggacctggaccttttagagagctctctcgccatcttttatctcccagagctgccatctaacaattgtcaaggaacctcatgtctcacctcaggggccagggta

SEQ ID NO: 141 - DNA, 410 bp, Mus musculus
ccaccaccgtccccagccccgctctgttccgggctgggactctaccccgggagcatcctccccagctgacatcatccatcccactccagccccaggctgctctgcccgccttgcacccagcgccgtcaacgccctgacccccggacctgccgctgccgctgcagacgccgccgcttcctccattgccaagggcggggcttagagctcaacccagacacctgtaggtgctggaagccgcgaaagtgacaagctgctttccagactccacgggcccggctgcttttatggccctgcttcacagggagaagagtggagcacaggcgaacctcctcagtctgggaggtcactgccccaggacctggaccttttagagagctctctcgccatcttttatctccagagctgcatct

SEQ ID NO: 142 - DNA, 566 bp, Homo sapiens
  unsure (508): May be any nucleic acid
ttccttcacatctcttttatcagggttgggggtcacagttcttgtaccaaagcccaaatcccattatgcagaggtttgggtcttcttagtgaggggaggaagagccagttgtaagatgcttacttgcacagagtggttagaaactgagacagtactccattctcccctgagctggtatgtgacccctcttgctgcctaccagtctcctcttccaactctgtcctgtttgatgatggcctcagggagacaagggatggcagaagagccctctgagaggcccaggtcctagagcagcttctgcctgggctgaggtcttgggggcttggctgtttaataccaggctcctctttgttcccccactgggatatagcctctgaggcaagtcaccctgctgagtctgaaaagccatgtgtcaccttcgcagcttcaggcacctgcaggtgtctaggttgagcactaagccccgcccttgcgaacggaggaagctgcggcgtcggcagcgcagtcnagagtccgggggtcagggcgctggatgatctgggtgcaggtgggcagaggggcctggg

SEQ ID NO: 143 - DNA, 88 bp, Homo sapiens
ttcgcagcttccggcacatgcaggtgtctgggttgagctctaagccccgcccttggcaacggaggaagctgcggttccggcagcggca

SEQ ID NO: 144 - DNA, 301 bp, Homo sapiens
tttttttttttttttttttttttttttttttttttttttttgccgcccactcaaactttattaaaagaccaggaaggggcgcgccaaaagcgaaagcccctcggggctcgcccccccgcctcaccgggtcagggaaaaaacgatcagagtaggggtatttcaccgggggcccgcagggccgggggaccccgccccgggccccttggggggacaccgggggggcgccgggggcctcccacttattctacacctttcatgttttttcaccgggccaaactagagtcaagctcaacagggtctt

SEQ ID NO: 145 - DNA, 594 bp, Rattus sp.
tcccggggagcccggcgggtcgccggcgcggggttttcctccggcctcgtcctcccccttccccctccgcggggtcgggggttcccggggttcggggttctcctccgcgtcggcggttcccccgccgggtgcgccccccgggccgcggtttcccgcgcggcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccccccgttccccgcgaggggtc

SEQ ID NO: 146 - DNA, 667 bp, Rattus sp.
cgctcccggggcagcccggagggtcgccggcgcggggttttcctccggcctcgtcctcccccttccccctccgcggggtcgggggttcccggggttcggggttctcctccgcgtcggcggttcccccgccgggtgcgccccccgggccgcggtttcccgcgcggcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaacaaattcaatgaagcgcgggtaaacggcgggagtaactatgactctcttaaggtagccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccccccgttccccgcgaggggtcggggcggggtccgccggccttcgcggccgccggtgaaataccactactctcatcgttttttcactgacc

SEQ ID NO: 147 - DNA, 522 bp, Rattus sp.
cctccgcggggtcgggggttcccggggttcggggttctcctccgcgtcggcggttcccccgccgggtgcgccccccgggccgcggtttcccgcgcggcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaagtgtagaataagtgggaggcccccggcgcccccccgttccccg

SEQ ID NO: 148 - DNA, 582 bp, Rattus sp.
cccggcgggtcgccggcgcggggttttcctccggcctcgtcctcccccttccccctccgcggggtcgggggttcccggggttcggggttctcctccgcgtcggcggttcccccgccgggtgcgccccccgggccgcggtttcccgcgcggcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtacaataagtgggaggcccccggcgcccccccgttccccgcgaaggg

SEQ ID NO: 149 - DNA, 586 bp, Rattus sp.
cacccggggagcccggcgggtcgccggcgcggggttttcctccggcctcgtcctcccccttccccctccgcggggtcgggggttcccggggttcggggttctcctccgcgtcggcggttcccccgccgggtgcgccccccgggccgcggtttcccgcgcggcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcctcgtcatctaattagtgacgcgcatgaatggatgaacgagattcccactgtccctacctactatccagcgaaaccacagccaagggaacaggcttggcggaatcagcggggaaagaagaccctgttgagcttgactctagtctggcacggtgaagagacatgagaggtgtagaataagtgggaggcccccggcgctcccccgttccccg

SEQ ID NO: 150 - DNA, 325 bp, Mus musculus
cggacgcttgggccagaagcgagagccctcgcgtgcgcccccccgcctcaccgggtcagtgaaaaaacgatgagagtagtggtattcaccggcggcccgagcgcgtgccccgaccccgacgcgaggacggggccgggcgccgggggcctcccacttattctacacctctcatgtctcttcaccgtgccagactagagtcaagctcaacagggtcttctttccccgctgattccgccaagcccgttcccttggctgtggtttcgctggatagtaggtagggacagtgggaatctcgttcatccattcatgcgcgtcactaattaga

SEQ ID NO: 151 - DNA, 331 bp, Mus musculus
gacggacgcttggcgccagaagcgagacccctcggccgcccccccgcctcaccgggtcagtgaaaaaacgatgagagtagtggtatttcaccggcggcccgcgaggccgcgtgccccgaccccgacgcgaggacggggccgggcgccgggggcctcccacttattctacacctctcatgtctcttcaccgtgccagactagagtcaagctcaacagggtcttctttccccgctgattccgccaagcccgttcccttggctgtggtttcgctggatagtaggtagggacagtgggaatctcgttcatccattcatgcgcgtcactaattaga

SEQ ID NO: 152 - DNA, 399 bp, Homo sapiens
cgagataagtgggaggcccccggcgcccccccggtgtccccgcgaggggcccggggcggggtccgccggccctgcgggccgccggtgaaataccactactctgatcgttttttcactgacccggtgaggcgggggggcgagccccgaggggctctcgcttctggcgccaagcgcccggccgcgcgccggccgggcgcgacccgctccggggacagtgccaggtggggagtttgactcctaggatttcagcggtctgtgggccagaaagcaggcaccagggctgacctcaaggccgtatcagagggccaagcagagatcttttggatacctgcttttcatcccacagggccttagagtcagaggtaaggtagcaacagagctagaatggggcaatgcact

SEQ ID NO: 153 - DNA, 489 bp, Homo sapiens
gcggccgccgctgccgctgtcgccgccgccgccgccaccgcgccaggttccggccgcggccaccctccgccgtccagggcccctccgtctcggccccgggaccccggctccccgccagccccggcccggccccggcaccatgtcggagaaaagcgtggaggcagcggccgagttgagcgccaaggacctgaaggagaagaaggagaaggtggaggagaaggcaagccggaaagagcgaaagaaagaagtggtggaggaggaggagaacggggctgaggaggaagaagaagaaactgccgaggatggagaggaggaagatgaaggggaagaagaagatgaggaagaagaagaagaggatgatgaagggcccgcgctgaagagagctgccgaagaggaggatgaagcggatcccaaacggcagaagacagaaaatggggcatcggcgtgagcccctgccaacaggctggggttgggaggcctctctgggcc

SEQ ID NO: 154 - protein, 17 aa, Homo sapiens
MSPLLRRLLLAALLQLA

SEQ ID NO: 155 - protein, 16 aa, Homo sapiens
MSPLLRRLLLAALLQL

SEQ ID NO: 156 - protein, 15 aa, Homo sapiens
MSPLLRRLLLAALLQ

SEQ ID NO: 157 - protein, 14 aa, Homo sapiens
MSPLLRRLLLAALL

SEQ ID NO: 158 - protein, 13 aa, Homo sapiens
MSPLLRRLLLAAL

SEQ ID NO: 159 - protein, 12 aa, Homo sapiens
MSPLLRRLLLAA

SEQ ID NO: 160 - protein, 11 aa, Homo sapiens
MSPLLRRLLLA

SEQ ID NO: 161 - protein, 10 aa, Homo sapiens
MSPLLRRLLL

SEQ ID NO: 162 - protein, 9 aa, Homo sapiens
MSPLLRRLL

SEQ ID NO: 163 - protein, 8 aa, Homo sapiens
MSPLLRRL

SEQ ID NO: 164 - protein, 7 aa, Homo sapiens
MSPLLRR

SEQ ID NO: 165 - protein, 6 aa, Homo sapiens
MSPLLR

SEQ ID NO: 166 - protein, 5 aa, Homo sapiens
MSPLL

SEQ ID NO: 167 - protein, 4 aa, Homo sapiens
MSPL

SEQ ID NO: 168 - protein, 16 aa, Homo sapiens
SPLLRRLLLAALLQLA

SEQ ID NO: 169 - protein, 17 aa, Homo sapiens
MSPLLRRLLLAALLQLA

SEQ ID NO: 170 - protein, 16 aa, Homo sapiens
MPLLRRLLLAALLQLA

SEQ ID NO: 171 - protein, 15 aa, Homo sapiens
PLLRRLLLAALLQLA

SEQ ID NO: 172 - protein, 15 aa, Homo sapiens
MLLRRLLLAALLQLA

SEQ ID NO: 173 - protein, 14 aa, Homo sapiens
LLRRLLLAALLQLA

SEQ ID NO: 174 - protein, 14 aa, Homo sapiens
MLRRLLLAALLQLA

SEQ ID NO: 175 - protein, 13 aa, Homo sapiens
LRRLLLAALLQLA

SEQ ID NO: 176 - protein, 13 aa, Homo sapiens
MRRLLLAALLQLA

SEQ ID NO: 177 - protein, 12 aa, Homo sapiens
RRLLLAALLQLA

SEQ ID NO: 178 - protein, 12 aa, Homo sapiens
MRLLLAALLQLA

SEQ ID NO: 179 - protein, 11 aa, Homo sapiens
RLLLAALLQLA

SEQ ID NO: 180 - protein, 11 aa, Homo sapiens
MLLLAALLQLA

SEQ ID NO: 181 - protein, 10 aa, Homo sapiens
LLLAALLQLA

SEQ ID NO: 182 - protein, 10 aa, Homo sapiens
MLLAALLQLA

SEQ ID NO: 183 - protein, 9 aa, Homo sapiens
LLAALLQLA

SEQ ID NO: 184 - protein, 9 aa, Homo sapiens
MLAALLQLA

SEQ ID NO: 185 - protein, 8 aa, Homo sapiens
LAALLQLA

SEQ ID NO: 186 - protein, 8 aa, Homo sapiens
MAALLQLA

SEQ ID NO: 187 - protein, 7 aa, Homo sapiens
AALLQLA

SEQ ID NO: 188 - protein, 7 aa, Homo sapiens
MALLQLA

SEQ ID NO: 189 - protein, 6 aa, Homo sapiens
ALLQLA

SEQ ID NO: 190 - protein, 6 aa, Homo sapiens
MLLQLA

SEQ ID NO: 191 - protein, 5 aa, Homo sapiens
LLQLA

SEQ ID NO: 192 - protein, 5 aa, Homo sapiens
MLQLA

SEQ ID NO: 193 - protein, 4 aa, Homo sapiens
LQLA

SEQ ID NO: 194 - protein, 4 aa, Homo sapiens
MQLA

(19) United States
(12) Patent Application Publication
(10) Pub. No.: US 2002/0026037 A1
(43) Pub. Date: Feb. 28, 2002

(54) VASCULAR ENDOTHELIAL GROWTH FACTOR 3 ANTIBODIES

(76) Inventors: JING-SHAN HU, SUNNYVALE, CA (US); HENRIK OLSEN, GAITHERSBURG, MD (US); CRAIG A. ROSEN, LAYTONSVILLE, MD (US)

Correspondence Address: STERNE KESSLER GOLDSTEIN & FOX, 1100 NEW YORK AVENUE N W, SUITE 600, WASHINGTON, DC, 200053934

(21) Appl. No.: 09/244,694

(22) Filed: Feb. 10, 1999

Related U.S. Application Data
(63) Continuation-in-part of application No. 09/132,088, filed on Aug. 10, 1998, now abandoned.
(63) Continuation-in-part of application No. 09/033,662, filed on Mar. 3, 1998.
(62) Division of application No. 08/469,641, filed on Jun. 6, 1995.

(51) Int. Cl. C12P 21/02; A61K 39/21; C07K 16/00

(52) U.S. Cl. 530/389.2; 424/187.1

(57) ABSTRACT

The present invention relates to a novel human protein called Vascular Endothelial Growth Factor 3, and isolated polynucleotides encoding this protein. Also provided are vectors, host cells, antibodies, and recombinant methods for producing this human protein. The invention further relates to diagnostic and therapeutic methods useful for diagnosing and treating disorders related to this novel human protein.

### CROSS-REFERENCE TO RELATED APPLICATIONS

The present application is a continuation-in-part of U.S. appl. Ser. No. 09/132,088, filed Aug. 10, 1998, which is herein incorporated by reference; said ser. No. 09/132,088 is a continuation-in-part of U.S. appl. Ser. No. 09/033,662, filed Mar. 3, 1998, which is herein incorporated by reference; said Ser. No. 09/033,662 is a divisional of U.S. appl. Ser. No. 08/469,641, filed Jun. 6, 1995, which is also herein incorporated by reference.

### BACKGROUND OF THE INVENTION

1. Field of the Invention

The present invention relates to a novel human gene encoding a polypeptide which is a member of the Vascular Endothelial Growth Factor family. More specifically, the present invention relates to a polynucleotide encoding a novel human polypeptide named Vascular Endothelial Growth Factor 3, or “VEGF-3.” This invention also relates to VEGF-3 polypeptides, as well as vectors, host cells, antibodies directed to VEGF-3 polypeptides, and the recombinant methods for producing the same. Also provided are diagnostic methods for detecting disorders related to the vascular and lymphatic system, and therapeutic methods for treating such disorders. The invention further relates to screening methods for identifying agonists and antagonists of VEGF-3 activity.

2. Related Art

The formation of new blood vessels, or angiogenesis, is essential for embryonic development, subsequent growth, and tissue repair. Angiogenesis, however, is an essential part of certain pathological conditions such as neoplasia, for example, tumors and gliomas, and abnormal angiogenesis is associated with other diseases such as inflammation, rheumatoid arthritis, psoriasis, and diabetic retinopathy (Folkman, J. and Klagsbrun, M., Science 235:442-447 (1987)).

Both acidic and basic fibroblast growth factor molecules are mitogens for endothelial cells and other cell types. Angiotropin and angiogenin can induce angiogenesis, although their functions are unclear (Folkman, J., 1993, Cancer Medicine pp. 153-170, Lea and Febiger Press). A highly selective mitogen for vascular endothelial cells is vascular endothelial growth factor or VEGF (Ferrara, N., et al., Endocr. Rev. 13:19-32 (1992)), also known as vascular permeability factor (VPF). Vascular endothelial growth factor is a secreted angiogenic mitogen whose target cell specificity appears to be restricted to vascular endothelial cells.

The murine VEGF gene has been characterized and its expression pattern in embryogenesis has been analyzed. A persistent expression of VEGF was observed in epithelial cells adjacent to fenestrated endothelium, e.g., in choroid plexus and kidney glomeruli. The data was consistent with a role of VEGF as a multifunctional regulator of endothelial cell growth and differentiation (Breier, G. et al., Development 114:521-532 (1992)).

VEGF is structurally related to the a and p chains of platelet-derived growth factor (PDGF), a mitogen for mesenchymal cells and placenta growth factor (PLGF), an endothelial cell mitogen. These three proteins belong to the same family and share a conserved motif. Eight cysteine residues contributing to disulfide-bond formation are strictly conserved in these proteins. Alternatively spliced mRNAs have been identified for both VEGF, PLGF and PDGF and these different splicing products differ in biological activity and in receptor-binding specificity. VEGF and PDGF function as homo-dimers or hetero-dimers and bind to receptors which elicit intrinsic tyrosine kinase activity following receptor dimerization.

VEGF has four different forms of 121, 165, 189 and 206 amino acids due to alternative splicing. VEGF 121 and VEGF 165 are soluble and are capable of promoting angiogenesis, whereas VEGF 189 and VEGF306 are bound to heparin containing proteoglycans in the cell surface. The temporal and spatial expression of VEGF has been correlated with physiological proliferation of the blood vessels (Gajdusek, C. M., and Carbon, S. J., Cell Physiol. 139:570-579 (1989)); McNeil, P. L., et al., J. Cell. Biol. 109:811-822 (1989)). Its high affinity binding sites are localized only on endothelial cells in tissue sections (Jakeman, L. B., et al., Clin. Invest. 89:244-253 (1989)). The factor can be isolated from pituitary cells and several tumor cell lines, and has been implicated in some human gliomas (Plate, K. H., Nature 359:845-848 (1992)). Interestingly, expression of VEGF121 or VEGF165 confers on Chinese hamster ovary cells the ability to form tumors in nude mice (Ferrara, N., et al., J. Clin. Invest. 91:160-170 (1993)). The inhibition of VEGF function by anti-VEGF monoclonal antibodies was shown to inhibit tumor growth in immune-deficient mice (Kim, K. J., Nature 362:841-844 (1993)). Further, a dominant-negative mutant of the VEGF receptor has been shown to inhibit growth of glioblastomas in mice.

Vascular permeability factor, has also been found to be responsible for persistent microvascular hyperpermeability to plasma proteins even after the cessation of injury, which is a characteristic feature of normal wound healing. This suggests that VPF is an important factor in wound healing (Brown, L. F. et al., J. Exp. Med. 176:1375-1379 (1992)).

The expression of VEGF is high in vascularized tissues, (e.g., lung, heart, placenta and solid tumors) and correlates with angiogenesis both temporally and spatially. VEGF has also been shown to induce angiogenesis in vivo. Since angiogenesis is essential for the repair of normal tissues, especially vascular tissues, VEGF has been proposed for use in promoting vascular tissue repair (e.g., in atherosclerosis).

U.S. Pat. No. 5,073,492, issued Dec. 17, 1991 to Chen et al., discloses a method for synergistically enhancing endothelial cell growth in an appropriate environment which comprises adding to the environment, VEGF, effectors and serum-derived factor. Also, vascular endothelial cell growth factor C sub-unit DNA has been prepared by polymerase chain reaction techniques. The DNA encodes a protein that may exist as either a hetero-dimer or homo-dimer. The protein is a mammalian vascular endothelial cell mitogen and, as such, is useful for the promotion of vascular development and repair, as disclosed in European Patent Application No. 92302750.2, published Sep. 30, 1992.

Thus, there is a need for polypeptides that promote growth of vessels, since disturbances of such regulation may be involved in disorders relating to the vascular and lymphatic system. Therefore, there is a need for identifying and characterizing human polypeptides which can play a role in detecting, preventing, ameliorating or correcting such disorders.

### SUMMARY OF THE INVENTION

The present invention relates to a novel polynucleotide and the encoded polypeptide of VEGF-3. Moreover, the present invention relates to vectors, host cells, antibodies, and recombinant methods for producing the polypeptides and polynucleotides. Also provided are diagnostic methods for detecting disorders relates to the polypeptides, and therapeutic methods for treating such disorders. The invention further relates to screening methods for identifying binding partners of VEGF-3.

### BRIEF DESCRIPTION OF THE FIGURES

FIG. 1 shows the nucleotide sequence (SEQ ID NO:1) and the deduced amino acid sequence (SEQ ID NO:2) of VEGF-3.

FIG. 2 shows the regions of identity between the amino acid sequence of the VEGF-3 protein (SEQ ID NO:2) and the translation product of the human VEGF (SEQ ID NO:3), determined by BLAST analysis. Identical amino acids between the two polypeptides have lines between them, while conservative amino acids have dots between them. By examining the regions of amino acids with lines and/or dots between them, the skilled artisan can readily identify conserved domains between the two polypeptides.

FIG. 3 shows an analysis of the VEGF-3 amino acid sequence (SEQ ID NO:2). Alpha, beta, turn and coil regions; hydrophilicity and hydrophobicity; amphipathic regions; flexible regions; antigenic index and surface probability are shown. In the “Antigenic Index or Jameson-Wolf” graph, the positive peaks indicate locations of the highly antigenic regions of the VEGF-3 protein, i.e., regions from which epitope-bearing peptides of the invention can be obtained. The domains defined by these graphs are contemplated by the present invention.

FIG. 4 shows the nucleotide sequence (SEQ ID NO:19) and the deduced amino acid sequence (SEQ ID NO:20) of a VEGF-3 splice variant.

FIG. 5 shows an analysis of the VEGF-3 splice variant amino acid sequence (SEQ ID NO:20). Alpha, beta, turn and coil regions; hydrophilicity and hydrophobicity; amphipathic regions; flexible regions; antigenic index and surface probability are shown. In the “Antigenic Index or Jameson-Wolf” graph, the positive peaks indicate locations of the highly antigenic regions of the VEGF-3 protein, i.e., regions from which epitope-bearing peptides of the invention can be obtained. The domains defined by these graphs are contemplated by the present invention.

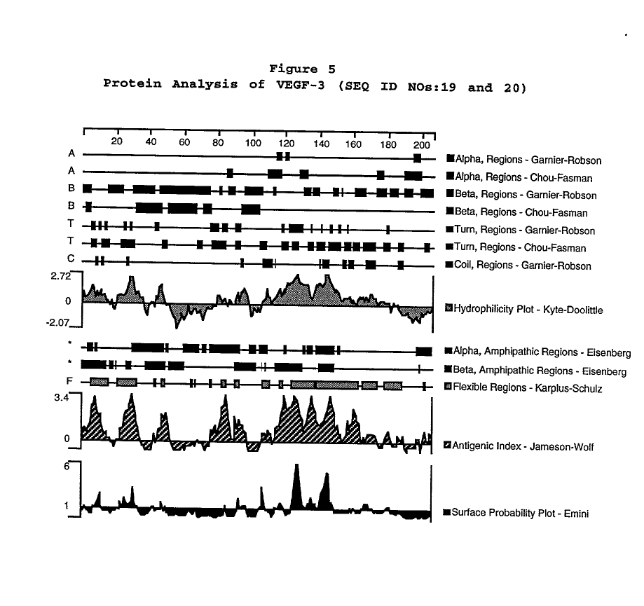

### DETAILED DESCRIPTION OF THE PREFERRED EMBODIMENTS

Definitions

The following definitions are provided to facilitate understanding of certain terms used throughout this specification.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide.

In the present invention, a “secreted” VEGF-3 protein refers to a protein capable of being directed to the ER, secretory vesicles, or the extracellular space as a result of a signal sequence, as well as a VEGF-3 protein released into the extracellular space without necessarily containing a signal sequence. If the VEGF-3 secreted protein is released into the extracellular space, the VEGF-3 secreted protein can undergo extracellular processing to produce a “mature” VEGF-3 protein. Release into the extracellular space can occur by many mechanisms, including exocytosis and proteolytic cleavage.

As used herein, a VEGF-3 “polynucleotide” refers to a molecule having a nucleic acid sequence contained in SEQ ID NO:1 or SEQ ID NO:19 or the cDNA contained within the clone deposited with the ATCC. For example, the VEGF-3 polynucleotide can contain the nucleotide sequence of the full length cDNA sequence, including the 5′ and 3′ untranslated sequences, the coding region, with or without the signal sequence, the secreted protein coding region, as well as fragments, epitopes, domains, and variants of the nucleic acid sequence. Moreover, as used herein, a VEGF-3 “polypeptide” refers to a molecule having the translated amino acid sequence generated from the polynucleotide as broadly defined.

In specific embodiments, the polynucleotides of the invention are less than 300 kb, 200 kb, 100 kb, 50 kb, 15 kb, 10 kb, or 7.5 kb in length. In a further embodiment, polynucleotides of the invention comprise at least 15 contiguous nucleotides of VEGF-3 coding sequence, but do not comprise all or a portion of any VEGF-3 intron. In another embodiment, the nucleic acid comprising VEGF-3 coding sequence does not contain coding sequences of a genomic flanking gene (i.e., 5′ or 3′ to the VEGF-3 gene in the genome).

In the present invention, the full length VEGF-3 sequence identified as SEQ ID NO:1 was generated by overlapping sequences contained in multiple clones (contig analysis). A representative clone containing all or most of the sequence for SEQ ID NO:1 was deposited with the American Type Culture Collection (“ATCC”) on May 26, 1995, and was given the ATCC Deposit Number 97166. The ATCC is located at 10801 University Boulevard, Manassas, Va. 20110-2209, USA. The ATCC deposit was made pursuant to the terms of the Budapest Treaty on the international recognition of the deposit of microorganisms for purposes of patent procedure.

A VEGF-3 “polynucleotide” also includes those polynucleotides capable of hybridizing, under stringent hybridization conditions, to sequences contained in SEQ ID NO:1 or SEQ ID NO:19, the complement thereof, or the cDNA within the deposited clone. “Stringent hybridization conditions” refers to an overnight incubation at 42° C. in a solution comprising 50% formamide, 5× SSC (750 mM NaCl, 75 mM sodium citrate), 50 mM sodium phosphate (pH 7.6), 5× Denhardt's solution, 10% dextran sulfate, and 20 μg/ml denatured, sheared salmon sperm DNA, followed by washing the filters in 0. 1× SSC at about 65° C.

Also contemplated are nucleic acid molecules that hybridize to the VEGF-3 polynucleotides at lower stringency hybridization conditions. Changes in the stringency of hybridization and signal detection are primarily accomplished through the manipulation of formamide concentration (lower percentages of formamide result in lowered stringency); salt conditions, or temperature. For example, lower stringency conditions include an overnight incubation at 37° C. in a solution comprising 6× SSPE (20× SSPE=3M NaCl; 0.2M NaH₂PO₄; 0.02M EDTA, pH 7.4), 0.5% SDS, 30% formamide, 100 μg/ml salmon sperm blocking DNA, followed by washes at 50° C. with 1× SSPE, 0.1% SDS. In addition, to achieve even lower stringency, washes performed following stringent hybridization can be done at higher salt concentrations (e.g. 5× SSC).

Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Of course, a polynucleotide which hybridizes only to polyA+sequences (such as any 3′ terminal polyA+tract of a cDNA), or to a complementary stretch of T (or U) residues, would not be included in the definition of “polynucleotide,” since such a polynucleotide would hybridize to any nucleic acid molecule containing a poly (A) stretch or the complement thereof (e.g., practically any double-stranded cDNA clone).

The VEGF-3 polynucleotide can be composed of any polyribonucleotide or polydeoxribonucleotide, which may be unmodified RNA or DNA or modified RNA or DNA. For example, VEGF-3 polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the VEGF-3 polynucleotides can be composed of triple-stranded regions comprising RNA or DNA or both RNA and DNA. VEGF-3 polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

VEGF-3 polypeptides can be composed of amino acids joined to each other bypeptide bonds or modified peptide bonds, i.e., peptide isosteres, and may contain amino acids other than the 20 gene-encoded amino acids. The VEGF-3 polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in the VEGF-3 polypeptide, including the peptide backbone, the amino acid side-chains and the amino or carboxyl termini. It will be appreciated that the same type of modification may be present in the same or varying degrees at several sites in a given VEGF-3 polypeptide. Also, a given VEGF-3 polypeptide may containmany types ofmodifications. VEGF-3 polypeptides may be branched, for example, as a result of ubiquitination, and they may be cyclic, with or without branching. Cyclic, branched, and branched cyclic VEGF-3 polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, 2nd Ed., T. E. Creighton, W. H. Freeman and Company, New York (1993); Posttranslational Covalent Modification of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

“SEQ ID NO:1” refers to a VEGF-3 polynucleotide sequence while “SEQ ID NO:2” refers to a VEGF-3 polypeptide sequence. “SEQ ID NO:19” refers to another VEGF-3 polynucleotide sequence while “SEQ ID NO:20” refers to the corresponding VEGF-3 polypeptide sequence.

A VEGF-3 polypeptide “having biological activity” refers to polypeptides exhibiting activity similar, but not necessarily identical to, an activity of a VEGF-3 polypeptide, including mature forms, as measured in a particular biological assay, with or without dose dependency. In the case where dose dependency does exist, it need not be identical to that of the VEGF-3 polypeptide, but rather substantially similar to the dose-dependence in a given activity as compared to the VEGF-3 polypeptide (i.e., the candidate polypeptide will exhibit greater activity or not more than about 25-fold less and, preferably, not more than about tenfold less activity, and most preferably, not more than about three-fold less activity relative to the VEGF-3 polypeptide.)

VEGF-3 Polynucleotides and Polypeptides

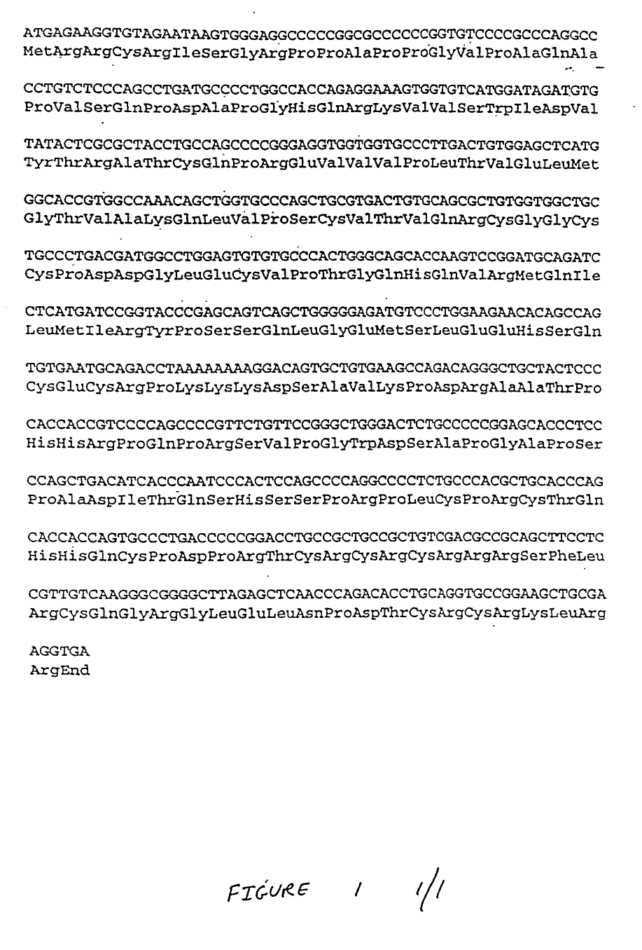

Clone HMWCF06 was isolated from a bone marrow cell line cDNA library. This clone contains the entire coding region identified as SEQ ID NO:2. The deposited clone contains a cDNA having a total of 666 nucleotides, which encodes a predicted open reading frame of 221 amino acid residues. (See FIG. 1.) The open reading frame begins at a N-terminal methionine located at nucleotide position 1, and ends at a stop codon at nucleotide position 664. Subsequent Northern analysis also showed VEGF-3 expression in colon, heart, kidney, and ovary tissues, a pattern consistent with vascular and lymphatic specific expression.

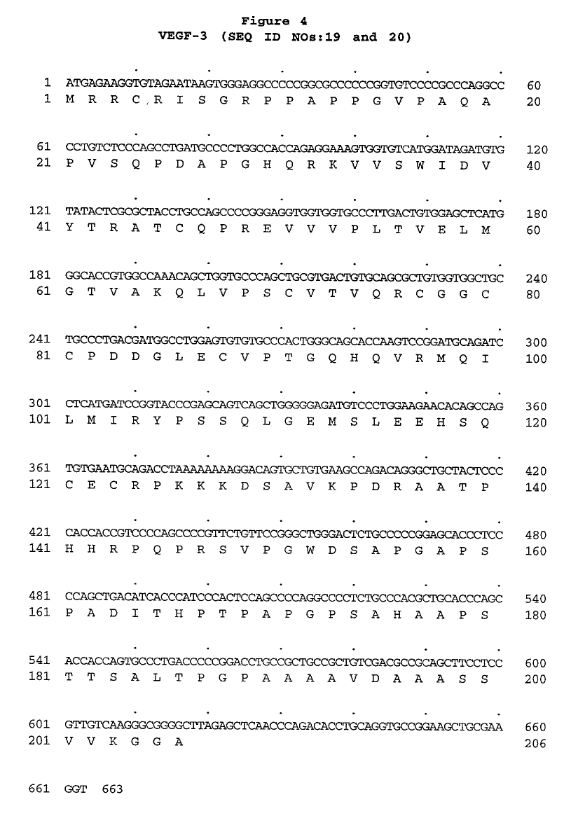

Another VEGF-3 polynucleotide sequence is shown in SEQ ID NO:19 (FIG. 4). It encodes a polypeptide of 206 amino acids (SEQ ID NO:20). The open reading frame begins at an N-terminal methionine located at nucleotide position 1 and ends at a stop codon at nucleotide position 618. In particular, SEQ ID NO:19 differs from SEQ ID NO:1 in that one nucleotide (“A”) at position 498 of SEQ ID NO:1 is not present in SEQ ID NO:19. As a result, SEQ ID NO:20 differs from SEQ ID NO:2 from residue 166 to the end of the molecules.

The VEGF-3 nucleotide sequence identified as SEQ ID NO:1 was assembled from partially homologous (“overlapping”) sequences obtained from the deposited clone. The overlapping sequences were assembled into a single contiguous sequence of high redundancy, resulting in a final sequence identified as SEQ ID NO:1.

Therefore, SEQ ID NO:1 and the translated SEQ ID NO:2 are sufficiently accurate and otherwise suitable for a variety of uses well known in the art and described further below. SEQ ID NO:19 and the translated SEQ ID NO:20 are also sufficiently accurate and otherwise suitable for a variety of uses well known in the art and described further below. For instance, SEQ ID NO:1 and SEQ ID NO:19 are useful for designing nucleic acid hybridization probes that will detect nucleic acid sequences contained in SEQ ID NO:1, SEQ ID NO:19 or the cDNA contained in the deposited clone. These probes will also hybridize to nucleic acid molecules in biological samples, thereby enabling a variety of forensic and diagnostic methods of the invention. Similarly, polypeptides identified from SEQ ID NO:2 and SEQ ID NO:20 may be used to generate antibodies which bind specifically to VEGF-3.

Nevertheless, DNA sequences generated by sequencing reactions can contain sequencing errors. The errors exist as misidentified nucleotides, or as insertions or deletions of nucleotides in the generated DNA sequence. The erroneously inserted or deleted nucleotides cause frame shifts in the reading frames of the predicted amino acid sequence. In these cases, the predicted amino acid sequence diverges from the actual amino acid sequence, even though the generated DNA sequence may be greater than 99.9% identical to the actual DNA sequence (for example, one base insertion or deletion in an open reading frame of over 1000 bases).

Accordingly, for those applications requiring precision in the nucleotide sequence or the amino acid sequence, the present invention provides not only the generated nucleotide sequence identified as SEQ ID NO:1 and the predicted translated amino acid sequence identified as SEQ ID NO:2 and SEQ ID NO:19 and the predicted translated amino acid sequence identified as SEQ ID NO:20, but also a sample of plasmid DNA containing a human cDNA of VEGF-3 deposited with the ATCC. The nucleotide sequence of the deposited VEGF-3 clone can readily be determined by sequencing the deposited clone in accordance with known methods. The predicted VEGF-3 amino acid sequence can then be verified from such deposits. Moreover, the amino acid sequence of the protein encoded by the deposited clone can also be directly determined by peptide sequencing or by expressing the protein in a suitable host cell containing the deposited human VEGF-3 cDNA, collecting the protein, and determining its sequence.

The present invention also relates to the VEGF-3 gene corresponding to SEQ ID NO:1, SEQ ID NO:2, SEQ ID NO:19, SEQ ID NO:20, or the deposited clone. The VEGF-3 gene can be isolated in accordance with known methods using the sequence information disclosed herein. Such methods include preparing probes or primers from the disclosed sequence and identifying or amplifying the VEGF-3 gene from appropriate sources of genomic material.

Also provided in the present invention are species homologs of VEGF-3. Species homologs may be isolated and identified by making suitable probes or primers from the sequences provided herein and screening a suitable nucleic acid source for the desired homologue.

The VEGF-3 polypeptides can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

The VEGF-3 polypeptides may be in the form of the secreted protein, including the mature form, or may be a part of a larger protein, such as a fusion protein (see below). It is often advantageous to include an additional amino acid sequence which contains secretory or leader sequences, pro-sequences, sequences which aid in purification, such as multiple histidine residues, or an additional sequence for stability during recombinant production.

VEGF-3 polypeptides are preferably provided in an isolated form, and preferably are substantially purified. A recombinantly produced version of a VEGF-3 polypeptide, including the secreted polypeptide, can be substantially purified by the one-step method described in Smith and Johnson, Gene 67:31-40 (1988). VEGF-3 polypeptides also can be purified from natural or recombinant sources using antibodies of the invention raised against the VEGF-3 protein in methods which are well known in the art.

Polynucleotide and Polypeptide Variants

“Variant” refers to a polynucleotide or polypeptide differing from the VEGF-3 polynucleotide or polypeptide, but retaining essential properties thereof. Generally, variants are overall closely similar, and, in many regions, identical to the VEGF-3 polynucleotide or polypeptide.

By a polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence encoding the VEGF-3 polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence shown of SEQ ID NO:1, the ORF (open reading frame), or any fragment specified as described herein.

As a practical matter, whether any particular nucleic acid molecule or polypeptide is at least 90%, 95%, 96%, 97%, 98% or 99% identical to a nucleotide sequence of the presence invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al., Comp. App. Biosci. 6:237-245 (1990). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, CutoffScore=1, Gap Penalty=5, Gap Size Penalty 0.05, Window Size=500 or the length of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3 ′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections are to made for the purposes of the present invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequences shown in SEQ ID NO:2 or to the amino acid sequence encoded by deposited DNA clone can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al., Comp. App. Biosci. 6:237-245 (1990). In a sequence alignment the query and subject sequences are either both nucleotide sequences or both amino acid sequences. The result of said global sequence alignment is in percent identity. Preferred parameters used in a FASTDB amino acid alignment are: Matrix—PAM 0, k-tuple=2, Mismatch Penalty=1, JoiningPenalty=20, RandomizationGroup Length=0, CutoffScore=1, Window Size=sequence length, Gap Penalty=5, Gap Size Penalty=0.05, Wndow Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are to made for the purposes of the present invention.

The VEGF-3 variants may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. VEGF-3 polynucleotide variants can be produced for a variety of reasons, e.g., to optimize codon expression for a particular host (change codons in the human mRNA to those preferred by a bacterial host such as E. coli).

Naturally occurring VEGF-3 variants are called “allelic variants,” and refer to one of several alternate forms of a gene occupying a given locus on a chromosome of an organism. (Genes II, Lewin, B., ed., John Wiley & Sons, New York (1985).) These allelic variants can vary at either the polynucleotide and/or polypeptide level. Alternatively, non-naturally occurring variants may be produced by mutagenesis techniques or by direct synthesis.

Using known methods of protein engineering and recombinant DNA technology, variants may be generated to improve or alter the characteristics of the VEGF-3 polypeptides. For instance, one or more amino acids can be deleted from the N-terminus or C-terminus of the secreted protein without substantial loss of biological function. The authors of Ron et al., J. Biol. Chem. 268: 2984-2988 (1993), reported variant KGF proteins having heparin binding activity even after deleting 3, 8, or 27 amino-terminal amino acid residues. Similarly, Interferon gamma exhibited up to ten times higher activity after deleting 8-10 amino acid residues from the carboxy terminus ofthis protein (Dobeli et al., J. Biotechnology 7:199-216 (1988)).

Moreover, ample evidence demonstrates that variants often retain a biological activity similar to that of the naturally occurring protein. For example, Gayle and coworkers (J. Biol. Chem 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See, Abstract.) In fact, only 23 unique amino acid sequences, out of more than 3,500 nucleotide sequences examined, produced a protein that significantly differed in activity from wild-type.

Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

Thus, the invention further includes VEGF-3 polypeptide variants which show substantial biological activity. Such variants include deletions, insertions, inversions, repeats, and substitutions selected according to general rules known in the art so as have little effect on activity. For example, guidance concerning how to make phenotypically silent amino acid substitutions is provided in Bowie, J. U. et al., Science 247: 1306-1310 (1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used. (Cunningham and Wells, Science 244:1081-1085 (1989).) The resulting mutant molecules can then be tested for biological activity.

As the authors state, these two strategies have revealed that proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

Besides conservative amino acid substitution, variants of VEGF-3 include (i) substitutions with one or more of the non-conserved amino acid residues, where the substituted amino acid residues may or may not be one encoded by the genetic code, or (ii) substitution with one or more of amino acid residues having a substituent group, or (iii) fusion of the mature polypeptide with another compound, such as a compound to increase the stability and/or solubility of the polypeptide (for example, polyethylene glycol), or (iv) fusion of the polypeptide with additional amino acids, such as an IgG Fc fusion region peptide, or leader or secretory sequence, or a sequence facilitating purification. Such variant polypeptides are deemed to be within the scope ofthose skilled in the art from the teachings herein.

For example, VEGF-3 polypeptide variants containing amino acid substitutions of charged amino acids with other charged or neutral amino acids may produce proteins with improved characteristics, such as less aggregation. Aggregation of pharmaceutical formulations both reduces activity and increases clearance due to the aggregate's immunogenic activity. (Pinckard et al., Clin. Exp. Immunol. 2:331-340 (1967); Robbins et al., Diabetes 36: 838-845 (1987); Cleland et al., Crit. Rev. Therapeutic Drug Carrier Systems 10:307-377 (1993).)

Polynucleotide and Polypeptide Fragments

In the present invention, a “polynucleotide fragment” refers to a short polynucleotide having a nucleic acid sequence contained in the deposited clone or shown in SEQ ID NO:1 or SEQ ID NO:19. The short nucleotide fragments are preferably at least about 15 nt, and more preferably at least about 20 nt, still more preferably at least about 30 nt, and even more preferably, at least about 40 nt in length. A fragment “at least 20 nt in length,” for example, is intended to include 20 or more contiguous bases from the cDNA sequence contained in the deposited clone or the nucleotide sequence shown in SEQ ID NO:1 or SEQ ID NO:19. These nucleotide fragments are useful as diagnostic probes and primers as discussed herein. Of course, larger fragments (e.g., 50, 150, 200, 250, 300, 350, 400, 450, 500, 550, 600, 650 nucleotides) are preferred.

Moreover, representative examples of VEGF-3 polynucleotide fragments include, for example, fragments having a sequence from about nucleotide number 1-50, 51-100, 101-150,151-200,201-250,251-300,301-350,351-400,401-450, 451-500, 501-550, 551-600, or 651 to the end of SEQ ID NO:1 or SEQ ID NO:19 or the cDNA contained in the deposited clone. In this context “about” includes the particularly recited ranges, larger or smaller by several (5, 4, 3, 2, or 1) nucleotides, at either terminus or at both termini. Preferably, these fragments encode a polypeptide which has biological activity. More preferably, these polynucleotides can be used as probes or primers as discussed herein.

In the present invention, a “polypeptide fragment” refers to a short amino acid sequence contained in SEQ ID NO:2 or SEQ ID NO:20 or encoded by the cDNA contained in the deposited clone. Protein fragments may be “free-standing,” or comprised within a larger polypeptide of which the fragment forms a part or region, most preferably as a single continuous region. Representative examples of polypeptide fragments of the invention, include, for example, fragments from about amino acid number 1-20, 21-40, 41-60, 61-80, 81-100, 102-120, 121-140, 141-160, 161-180, 181-200, or 201 to the end of the coding region. Moreover, polypeptide fragments can be about 20, 30, 40, 50, 60, 70, 80, 90,100, 110, 120, 130, 140, or 150 amino acids in length. In this context “about” includes the particularly recited ranges, larger or smaller by several (5, 4, 3, 2, or 1) amino acids, at either extreme or at both extremes.

Preferred polypeptide fragments include the secreted VEGF-3 protein as well as the mature form. Further preferred polypeptide fragments include the secreted VEGF-3 protein or the mature form having a continuous series of deleted residues from the amino or the carboxy terminus, or both. For example, any number of amino acids, ranging from 1-60, can be deleted from the amino terminus of either the secreted VEGF-3 polypeptide or the mature form. Similarly, any number of amino acids, ranging from 1-30, can be deleted from the carboxy terminus of the secreted VEGF-3 protein or mature form. Furthermore, any combination of the above amino and carboxy terminus deletions are preferred. Similarly, polynucleotide fragments encoding these VEGF-3 polypeptide fragments are also preferred.

As mentioned above, even if deletion of one or more amino acids from the N-terminus of a protein results in modification of loss of one or more biological functions of the protein, other biological activities may still be retained. Thus, the ability of shortened VEGF-3 muteins to induce and/or bind to antibodies which recognize the complete or mature forms of the polypeptides generally will be retained when less than the majority of the residues of the complete or mature polypeptide are removed from the N-terminus. Whether a particular polypeptide lacking N-terminal residues of a complete polypeptide retains such immunologic activities can readily be determined by routine methods described herein and otherwise known in the art. It is not unlikely that a VEGF-3 mutein with a large number of deleted N-terminal amino acid residues may retain some biological or immunogenic activities. In fact, peptides composed of as few as six VEGF-3 amino acid residues may often evoke an immune response.

Accordingly, the present invention further provides polypeptides having one or more residues deleted from the amino terminus of the VEGF-3 amino acid sequence shown in SEQ ID NO:2, up to the cysteine residue at position number 216 and polynucleotides encoding such polypeptides. In particular, the present invention provides polypeptides comprising the amino acid sequence of residues n¹-221 of SEQ ID NO:2, where n¹ is an integer in the range of 17 to 216, and 216 is the position of the first residue from the N-terminus of the complete VEGF-3 polypeptide believed to be required for at least immunogenic activity of the VEGF-3 polypeptide.

More in particular, the invention provides polynucleotides encoding polypeptides comprising, or alternatively consisting of, the amino acid sequence of residues of P-17 to R-221; A-18 to R-221; Q-19 to R-221; A-20 to R-221; P-21 to R-221; V-22 to R-221; S-23 to R-221; Q-24 to R-221; P-25 to R-221; D-26 to R-221; A-27 to R-221; P-28 to R-221; G-29 to R-221; H-30 to R-221; Q-31 to R-221; R-32 to R-221; K-33 to R-221; V-34 to R-221; V-35 to R-221; S-36 to R-221; W-37 to R-221; 1-38 to R-221; D-39 to R-221; V-40 to R-221; Y-41 to R-221; T-42 to R-221; R-43 to R-221; A-44 to R-221; T-45 to R-221; C-46 to R-221; Q-47 to R-221; P-48 to R-221; R-49 to R-221; E-50 to R-221; V-51 to R-221; V-52 to R-221; V-53 to R-221; P-54 to R-221; L-55 to R-221; T-56 to R-221; V-57 to R-221; E-58 to R-221; L-59 to R-221; M-60 to R-221; G-61 to R-221; T-62 to R-221; V-63 to R-221; A-64 to R-221; K-65 to R-221; Q-66 to R-221; L-67 to R-221; V-68 to R-221; P-69 to R-221; S-70 to R-221; C-71 to R-221; V-72 to R-221; T-73 to R-221; V-74 to R-221; Q-75 to R-221; R-76 to R-221; C-77 to R-221; G-78 to R-221; G-79 to R-221; C-80 to R-221; C-81 to R-221; P-82 to R-221; D-83 to R-221; D-84 to R-221; G-85 to R-221; L-86 to R-221; E-87 to R-221; C-88 to R-221; V-89 to R-221; P-90 to R-221; T-91 to R-221; G-92 to R-221; Q-93 to R-221; H-94 to R-221; Q-95 to R-221 V-96 to R-221; R-97 to R-221; M-98 to R-221; Q-99 to R-221; 1-100 to R-221; L-101 to R-221; M-102 to R-221; 1-103 to R-221; R-104 to R-221; Y-105 to R-221;P-106 to R-221 ; S-107 to R-221;S-108 to R-221; Q-109 to R-221; L-110 to R-221; G-111 to R-221; E-112 to R-221; M-113 to R-221; S-114 to R-221; L-101 to R-221; E-116 to R-221; E-117 to R-221; H-118 to R-221; S-119 to R-221; Q-120 to R-221; C-121 to R-221; E-122 to R-221; C-123 to R-221R-124 to R-221; P-125 to R-221; K-126 to R-221; K-127 to R-221; K-128 to R-221; D-129 to R-221; S-130 to R-221; A-131 to R-221; V-132 to R-221; K-133 to R-221; P-134 to R-221; D-135 to R-221; R-136 to R-221; A-137 to R-221; A-138 to R-221; T-139 to R-221; P-140 to R-221; H-141 to R-221; H-142 to R-221; R-143 to R-221; P-144 to R-221; Q-145 to R-221; P-146 to R-221; R-147 to R-221; S-148 to R-221; V-149 to R-221; P-150 to R-221; G-151 to R-221; W-152 to R-221; D-153 to R-221; S-154 to R-221; A-155 to R-221;P-156 to R-221; G-157 to R-221; A-158 to R-221;P-159 to R-221; S-160 to R-221; P-161 to R-221; A-162 to R-221; D-163 to R-221; I-164 to R-221; T-165 to R-221; Q-166 to R-221; S-167 to R-221; H-168 to R-221; S-169 to R-221; S-170 to R-221; P-171 to R-221;R-172 to R-221;P-173 to R-221;L-174 to R-221; C-175 to R-221; P-176 to R-221; R-177 to R-221; C-178 to R-221; T-179 to R-221; Q-180 to R-221; H-181 to R-221; H-182 to R-221; Q-183 to R-221; C-184 to R-221;P-185 to R-221; D-186 to R-221; P-187 to R-221;R-188 to R-221; T-189 to R-221; C-190 to R-221; R-191 to R-221; C-192 to R-221; R-193 to R-221; C-194 to R-221; R-195 to R-221; R-196 to R-221; R-197 to R-221; S-198 to R-221;F-199 to R-221;L-200 to R-221;R-201 to R-221; C-202 to R-221; Q-203 to R-221; G-204 to R-221; R-205 to R-221; G-206 to R-221; L-207 to R-221; E-208 to R-221; L-209 to R-221; N-210 to R-221; P-211 to R-221; D-212 to R-221; T-213 to R-221; C-214 to R-221; R-215 to R-221; and C-216 to R-221 of the VEGF-3 sequence shown in SEQ ID NO:2. Polynucleotides encoding these polypeptides are also encompassed by the invention.

Also as mentioned above, even if deletion of one or more amino acids from the C-terminus of a protein results in modification of loss of one or more biological functions of the protein, other biological activities may still be retained. Thus, the ability of the shortened VEGF-3 mutein to induce and/or bind to antibodies which recognize the complete or mature forms of the polypeptide generally will be retained when less than the majority of the residues of the complete or mature polypeptide are removed from the C-terminus. Whether a particular polypeptide lacking C-terminal residues of a complete polypeptide retains such immunologic activities can readily be determined by routine methods described herein and otherwise known in the art. It is not unlikely that a VEGF-3 mutein with a large number of deleted C-terminal amino acid residues may retain some biological or immunogenic activities. In fact, peptides composed of as few as six VEGF-3 amino acid residues may often evoke an immune response.

Accordingly, the present invention further provides polypeptides having one or more residues deleted from the carboxy terminus of the amino acid sequence of the VEGF-3 polypeptide shown in SEQ ID NO:2, up to the valine residue at position number 22, and polynucleotides encoding such polypeptides. In particular, the present invention provides polypeptides comprising the amino acid sequence of residues 17-m¹ of SEQ ID NO:2, where m¹ is an integer in the range of 22 to 221, and 22 is the position of the first residue from the C-terminus of the complete VEGF-3 polypeptide believed to be required for at least immunogenic activity of the VEGF-3 polypeptide.

More in particular, the invention provides polynucleotides encoding polypeptides comprising, or alternatively consisting of, the amino acid sequence of residues P-17 to R-221; P-17 to R-220; P-17 to L-219; P-17 to K-218; P-17 to R-217; P-17 to C-216; P-17 to R-215; P-17 to C-214; P-17 to T-213; P-17 to D-212; P-17 to P-211; P-17 to N-210; P-17 to L-209; P-17 to E-208; P-17 to L-207; P-17 to G-206; P-17 to R-205; P-17 to G-204; P-17 to Q-203; P-17 to C-202; P-17 to R-201; P-17 to L-200; P-17 to F-199; P-17 to S-198; P-17 to R-197; P-17 to R-196; P-17 to R-195; P-17 to C-194; P-17 to R-193; P-17 to C-192; P-17 to R-191; P-17 to C-190; P-17 to T-189; P-17 to R-188; P-17 to P-187; P-17 to D-186; P-17 to P-185; P-17 to C-184; P-17 to Q-183; P-17 to H-182; P-17 to H-181; P-17 to Q-180; P-17 to T-179; P-17 to C-178; P-17 to R-177; P-17 to P-176; P-17 to C-175; P-17 to L-174; P-17 to P-173; P-17 to R-172;P-17 to P-171; P-17 to S-170; P-17 to S-169; P-17 to H-168; P-17 to S-167; P-17 to Q-166; P-17 to T-165; P-17 to 1-164; P-17 to D-163; P-17 to A-162; P-17 to P-161; P-17 to S-160; P-17 to P-159; P-17 to A-158; P-17 to G-157; P-17 to P-156; P17 to A-55; P-17 to S-154; P17 to D-153; P-17 to W-152; P-17 to G-151; P-17 to P-150; P-17 to V-149; P-17 to S-148; P-17 to R-147; P-17 to P-146; P-17 to Q-145; P-17 to P-144; P-17 to R-143; P-17 to H-142; P-17 to H-141; P-17 to P-140; P-17 to T-139; P-17 to A-138; P-17 to A-137; P-17 to R-136; P-17 to D-135; P-17 to P-134; P-17 to K-133; P-17 to V-132; P-17 to A-131; P-17 to P-130; P-17 to D-129; P-17 to K-128; P-17 to K-127; P-17 to K-126; P-17 to P-125; P-17 to R-124; P-17 to C-123; P-17 to E-122; P-17 to C-121; P-17 to Q-120; P-17 to S-119; P-17 to H-118; P-17 to E-117; P-17 to E-116; P-17 to L-115; P-17 to S-114; P-17 to P-1713; P-17 to E-112; P-17 to G-111; P-17 to L-110; P-17 to Q-109; P-17 to S-108; P-17 to S-107; P-17 to P-106; P-17 to Y-105; P-17 to R-104; P-17 to I-103; P-17 to P-1702; P-17 to L-101; P-17 to I-100; P-17 to Q-99;P-17 to M-98;P-17 to R-97; P-17 to V-96; P-17 to Q-95; P-17 to H-94; P-17 to Q-93; P-17 to G-92; P-17 to T-91; P-17 to P-90; P-17 to V-89; P-17 to C-88; P-17 to E-87; P-17 to L-86; P-17 to G-85; P-17 to D-84; P-17 to D-83; P-17 to P-82; P-17 to C-81; P-17 to C-80; P-17 to G-79; P-17 to G-78; P-17 to C-77; P-17 to R-76; P-17 to Q-75; P-17 to V-74; P-17 to T-73; P-17 to V-72; P-17 to C-71; P-17 to S-70; P-17 to P-69; P-17 to V-68; P-17 to L-67; P-17 to Q-66; P-17 to K-65; P-17 to A-64; P-17 to V-63; P-17 to T-62; P-17 to G-61; P-17 to M-60; P-17 to L-59; P-17 to E-58; P-17 to V-57; P-17 to T-56; P-17 to L-55; P-17 to P-54; P-17 to V-53; P-17 to V-52; P-17 to V-51; P-17 to E-50; P-17 to R-49; P-17 to P-48; P-17 to Q-47; P-17 to C-46; P-17 to T-45; P-17 to A-44; P-17 to R-43; P-17 to T-42; P-17 to Y-41; P-17 to V-40; P-17 to D-39; P-17 to 1-38; P-17 to W-37; P-17 to S-36; P-17 to V-35; P-17 to V-34; P-17 to K-33; P-17 to R-32; P-17 to Q-31; P-17 to H-30; P-17 to G-29; P-17 to P-28; P-17 to A-27; P-17 to D-26; P-17 to P-25; P-17 to Q-24; P-17 to S-23; and P-17 to V-22 of the sequence of the VEGF-3 sequence shown in SEQ ID NO:2. Polynucleotides encoding these polypeptides also are provided.

The invention also provides polypeptides having one or more amino acids deleted from both the amino and the carboxyl termini of a VEGF-3 polypeptide, which may be described generally as having residues n¹-m¹ of SEQ ID NO:2, where n¹ and m¹ are integers as described above.

The present invention also provides polypeptides having one or more residues deleted from the amino terminus of the VEGF-3 amino acid sequence shown in SEQ ID NO:20, up to the valine residue at position number 201 and polynucleotides encoding such polypeptides. In particular, the present invention provides polypeptides comprising the amino acid sequence of residues n²-206 of SEQ ID NO:20, where n² is an integer in the range of 17 to 201, and 201 is the position of the first residue from the N-terminus of the complete VEGF-3 polypeptide believed to be required for at least immunogenic activity of the VEGF-3 polypeptide.

More in particular, the invention provides polynucleotides encoding polypeptides comprising, or alternatively consisting of, the amino acid sequence of residues of P-17 to A-206; A-18 to A-206; Q-19 to A-206; A-20 to A-206; P-21 to A-206; V-22 to A-206; S-23 to A-206; Q-24 to A-206; P-25 to A-206; D-26 to A-206; A-27 to A-206; P-28 to A-206; G-29 to A-206; H-30 to A-206; Q-31 to A-206; R-32 to A-206; K-33 to A-206; V-34 to A-206; V-35 to A-206; S-36 to A-206; W-37 to A-206; I-38 to A-206; D-39 to A-206; V-40 to A-206; Y-41 to A-206; T-42 to A-206; R-43 to A-206; A-44 to A-206; T-45 to A-206; C-46 to A-206; Q-47 to A-206; P-48 to A-206; R-49 to A-206; E-50 to A-206; V-51 to A-206; V-52 to A-206; V-53 to A-206; P-54 to A-206; L-55 to A-206; T-56 to A-206; V-57 to A-206; E-58 to A-206; L-59 to A-206; M-60 to A-206; G-61 to A-206; T-62 to A-206; V-63 to A-206; A-64 to A-206; K-65 to A-206; Q-66 to A-206; L-67 to A-206; V-68 to A-206; P-69 to A-206; S-70 to A-206; C-71 to A-206; V-72 to A-206; T-73 to A-206; V-74 to A-206; Q-75 to A-206; R-76 to A-206; C-77 to A-206; G-78 to A-206; G-79 to A-206; C-80 to A-206; C-81 to A-206; P-82 to A-206; D-83 to A-206; D-84 to A-206; G-85 to A-206; L-86 to A-206; E-87 to A-206; C-88 to A-206; V-89 to A-206; P-90 to A-206; T-91 to A-206; G-92 to A-206; Q-93 to A-206; H-94 to A-206; Q-95 to A-206; V-96 to A-206; R-97 to A-206; M-98 to A-206; Q-99 to A-206; I-100 to A-206; L-101 to A-206; M-102 to A-206; 1-103 to A-206; R-104 to A-206; Y-105 to A-206; P-106 to A-206; S-107 to A-206; S-108 to A-206; Q-109 to A-206; L-110 to A-206; G-111 to A-206; E-112 to A-206; M-113 to A-206; S-114 to A-206; L-115 to A-206; E-116 to A-206; E-117 to A-206; H-118 to A-206; S-119 to A-206; Q-120 to A-206; C-121 to A-206; E-122 to A-206; C-123 to A-206; R-124 to A-206; P-125 to A-206; K-126 to A-206; K-127 to A-206; K-128 to A-206; D-129 to A-206; S-130 to A-206; A-131 to A-206; V-132 to A-206; K-133 to A-206; P-134 to A-206; D-135 to A-206; R-136 to A-206; A-137 to A-206; A-138 to A-206; T-139 to A-206; P-140 to A-206; H-141 to A-206; H-142 to A-206; R-143 to A-206; P-144 to A-206; Q-145 to A-206; P-146 to A-206; R-147 to A-206; S-148 to A-206; V-149 to A-206; P-150 to A-206; G-151 to A-206; W -152 to A-206; D-153 to A-206; S-154 to A-206; A-155 to A-206; P-156 to A-206; G-157 to A-206; A-158 to A-206; P-159 to A-206; S-160 to A-206; P-161 to A-206; A-162 to A-206; D-163 to A-206; I-164 to A-206; T-165 to A-206; H-166 to A-206; P-167 to A-206; T-168 to A-206; P-169 to A-206; A-170 to A-206; P-171 to A-206; G-172 to A-206; P-173 to A-206; S-174 to A-206; A-175 to A-206; H-176 to A-206; A-177 to A-206; A-178 to A-206; P-179 to A-206; S-180 to A-206; T-181 to A-206; T-182 to A-206; S-183 to A-206; A-184 to A-206; L-185 to A-206; T-186 to A-206; P-187 to A-206; G-188 to A-206; P-189 to A-206; A-190 to A-206; A-191 to A-206; A-192 to A-206; A-193 to A-206; V-194 to A-206; D-195 to A-206; A-196 to A-206; A-197 to A-206; A-198 to A-206; S-199 to A-206; S-200 to A-206; and V-201 to A-206 of the VEGF-3 sequence shown in SEQ ID NO:20. Polynucleotides encoding these polypeptides are also encompassed by the invention.

The present invention also provides polypeptides having one or more residues deleted from the carboxy terminus of the amino acid sequence of the VEGF-3 polypeptide shown in SEQ ID NO:20, up to the valine residue at position number 22, and polynucleotides encoding such polypeptides. In particular, the present invention provides polypeptides comprising the amino acid sequence of residues 17-m² of SEQ ID NO:20, where m² is an integer in the range of 22 to 206, and 22 is the position of the first residue from the C-terminus of the complete VEGF-3 polypeptide believed to be required for at least immunogenic activity of the VEGF-3 polypeptide.

More in particular, the invention provides polynucleotides encoding polypeptides comprising, or alternatively consisting of, the amino acid sequence of residues P-17 to A-206; P-17 to G-205; P-17 to G-204; P-17 to K-203; P-17 to V-202; P-17 to V-201; P-17 to S-200; P-17 to S-199; P-17 to A-198; P-17 to A-197; P-17 to A-196; P-17 to D-195; P-17 to V-194; P-17 to A-193; P-17 to A-192; P-17 to A-191; P-17 to A-190; P-17 to P-189; P-17 to G-188; P-17 to P-187; P-17 to T-186; P-17 to L-185; P-17 to A-184; P-17 to S-183; P-17 to T-182; P17 to T-181;P-17 to S-180; P-17 to P-179; P-17 to A-78; P17 to A-177; P-17 to H-176; P-17 to A-175; P-17 to S-174; P-17 to P-173; P-17 to A-172; P-17 to P-171; P-17 to A-170; P-17 to P-169; P-17 to T-168; P-17 to P-167; P-17 to H-166; P-17 to T-165; P-17 to 1-164; P-17 to D-163; P-17 to A-162; P-17 to P-161; P-17 to S-160; P-17 to P-159; P-17 to A-158; P-17 to G-157; P-17 to P-156; P-17 to A-155; P-17 to S-154; P-17 to D-153; P-17 to W-152; P-17 to G-151; P-17 to P-155; P-17 to V-149; P-17 to S-148; P-17 to R-147; P-17 to P-146; P-17 to Q-145; P-17 to P-144; P-17 to R-143; P-17 to R-142; P-17 to P-141; P-17 to P-140; P-17 to T-139; P-17 to A-138; P-17 to A-137; P-17 to R-136; P-17 to D-135; P-17 to P-134; P-17 to K-133; P-17 to V-132; P-17 to A-131; P-17 to S-130; P-17 to D-129; P-17 to K-128; P-17 to K-127; P-17 to K-126; P-17 to P-125; P-17 to R-124; P-17 to C-123; P-17 to E-122; P-17 to C-121; P-17 to Q-120; P-17 to S-119; P-17 to H-118; P-17 to E-117; P-17 to E-116; P-17 to L-115; P-17 to S-114; P-17 to P-1713; P-17 to E-112; P-17 toG-111; P-17 to L-110; P-17 to Q-109; P-17 to S-108; P-17 to S-107; P-17 to P-106; P-17 to Y-105; P-17 to R-104; P-17 to 1-103; P-17 to P-1702; P-17 to L-101; P-17 to 1-100; P-17 to Q-99; P-17 to M-98; P-17 to R-97; P-17 to V-96; P-17 to Q-95; P-17 to H-94; P-17 to Q-93; P-17 to G-92; P-17 to T-91; P-17 to P-90; P-17 to V-89; P-17 to C-88; P-17 to E-87; P-17 to L-86; P-17 to G-85; P-17 to D-84; P-17 to D-83; P-17 to P-82; P-17 to C-81; P-17 to C-80; P-17 to G-79; P-17 to G-78; P-17 to C-77; P-17 to R-76; P-17 to Q-75; P-17 to V-74; P-17 to T-73; P-17 to V-72; P-17 to C-71; P-17 to S-70; P-17 to P-69; P-17 to V-68; P-17 to L-67; P-17 to Q-66; P-17 to K-65; P-17 to A-64; P-17 to V-63; P-17 to T-62; P-17 to G-61; P-17 to M-60; P-17 to L-59; P-17 to E-58; P-17 to V-57; P-17 to T-56; P-17 to L-55; P-17 to P-54; P-17 to V-53; P-17 to V-52; P-17 to V-51; P-17 to E-50; P-17 to R-49; P-17 to P-48; P-17 to Q-47; P-17 to C-46; P-17 to T-45; P-17 to A-44; P-17 to R-43; P-17 to T-42; P-17 to Y-41; P-17 to V-40; P-17 to D-39; P-17 to 1-38; P-17 to W-37; P-17 to S-36; P-17 to V-35; P-17 to V-34; P-17 to K-33; P-17 to R-32; P-17 to Q-31; P-17 to H-30; P-17 to G-29; P-17 to P-28; P-17 to A-27; P-17 to D-26; P-17 to P-25; P-17 to Q-24; P-17 to S-23; and P-17 to V-22 of the sequence of the VEGF-3 sequence shown in SEQ ID NO:20. Polynucleotides encoding these polypeptides also are provided.

The invention also provides polypeptides having one or more amino acids deleted from both the amino and the carboxyl termini of a VEGF-3 polypeptide, which may be described generally as having residues n²-m² of SEQ ID NO:20, where n² and m² are integers as described above.

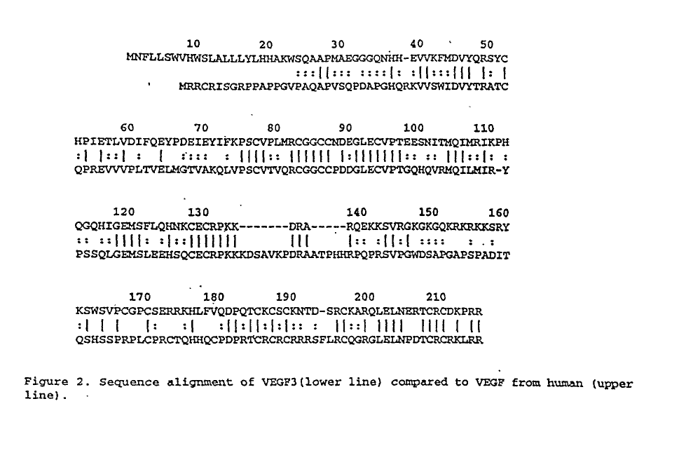

Particularly, N-terminal deletions of the VEGF-3 polypeptide can be described by the general formula m-221, where m is an integer from 2 to 215, where m corresponds to the position of the amino acid residue identified in SEQ ID NO:2. Preferably, N-terminal deletions retain the conserved region shown in FIG. 2 (PXCVXXXRCXGCCN)(SEQ ID NO:4), and includes polypeptides comprising the amino acid sequence of residues:: P-17 to R-221; A-18 to R-221; Q-19 to R-221; A-20 to R-221; P-21 to R-221; V-22 to R-221; S-23 to R-221; Q-24 to R-221; P-25 to R-221; D-26 to R-221; A-27 to R-221; P-28 to R-221; G-29 to R-221; H-30 to R-221; Q-31 to R-221; R-32 to R-221; K-33 to R-221; V-34 to R-221; V-35 to R-221; S-36 to R-221; W-37 to R-221; I-38 to R-221; D-39 to R-221; V-40 to R-221; Y-41 to R-221; T-42 to R-221; R-43 to R-221; A-44 to R-221; T-45 to R-221; C-46 to R-221; Q-47 to R-221; P-48 to R-221; R-49 to R-221; E-50 to R-221; V-51 to R-221; V-52 to R-221; V-53 to R-221; P-54 to R-221; L-55 to R-221; T-56 to R-221; V-57 to R-221; E-58 to R-221; L-59 to R-221; M-60 to R-221; G-61 to R-221; T-62 to R-221; V-63 to R-221; A-64 to R-221; K-65 to R-221; Q-66 to R-221; L-67 to R-221; V-68 to R-221; P-69 to R-221 of SEQ ID NO:2.

Moreover, C-terminal deletions of the VEGF-3 polypeptide can also be described by the general formula 1−n, where n is an integer from 15 to 221 where n corresponds to the position of amino acid residue identified in SEQ ID NO:2. Preferably, C-terminal deletions retain the conserved region shown in FIG. 2 (PXCVXXXRCXGCCN)(SEQ ID NO:4), and include polypeptides comprising the amino acid sequence of residues: P-17 to R-220; P-17 to L-219; P-17 to K-218; P-17 to R-217; P-17 to C-216; P-17 to R-215; P-17 to C-214; P-17 to T-213; P-17 to D-212; P-17 to P-211; P-17 to N-210; P-17 to L-209; P-17 to E-208; P-17 to L-207; P-17 to G-206; P-17 to R-205; P-17 to G-204; P-17 to Q-203; P-17 to C-202; P-17 to R-201; P-17 to L-200; P-17 to F-199; P-17 to S-198; P17 to R-197; P17 to R-196; P-17 to R-195; P-17 to C-194; P17 to R-193; P-17 to C-192; P-17 to R-191; P-17 to C-190; P-17 to T-189; P-17 to R-188; P-17 to P-187; P-17 to D-186; P-17 to P-185; P-17 to C-184; P-17 to Q-183; P-17 to H-182; P-17 to H-181; P-17 to Q-180; P-17 to T-179; P-17 to C-178; P-17 to R-177; P-17 to P-176; P-17 to C-175; P-17 to L-174; P-17 to P-173; P-17 to R-172; P-17 to P-171; P-17 to C-170; P-17 to L-169; P-17 to H-168; P-17 to R-167; P-17 to Q-166; P-17 to T-165; P-17 to 1-164; P-17 to D-163; P-17 to A-162; P-17 to P-166; P-17 to S-160; P-17 to P-159; P-17 to A-158; P-17 to G-157; P-17 to P-156; P-17 to A-155; P-17 to P-154; P-17 to D-153; P-17 to W-152; P-17 to G-151; P-17 to P-155; P-17 to V-149; P-17 to S-148; P-17 to R-147; P-17 to P-146; P-17 to Q-145; P-17 to P-144; P-17 to R-143; P-17 to H-142; P-17 to H-141; P-17 to P-140; P-17 to T-139; P-17 to A-138; P-17 to A-137; P-17 to R-136; P-17 to D-135; P-17 to P-134; P-17 to K-133; P-17 to V-132; P-17 to A-131; P-17 to S-130; P-17 to D-129; P-17 to K-128; P-17 to K-127; P-17 to K-126; P-17 to P-125; P-17 to R-124; P-17 to C-123; P-17 to E-122; P-17 to C-121; P-17 to Q-120; P-17 to S-119; P-17 to H-118; P-17 to E-117; P-17 to E-116; P-17 to L-115; P-17 to S-114; P-17 to M-113; P-17 to E-112; P-17 to G-111; P-17 to L-101; P-17 to Q-109; P-17 to S-108; P-17 to S-107; P-17 to P-106; P-17 to Y-105; P-17 to R-104; P-17 to I-103; P-17 to M-102; P-17 to L-101; P-17 to I-100; P-17 to Q-99; P-17 to M-98; P-17 to R-97; P-17 to V-96; P-17 to Q-95; P-17 to H-94; P-17 to Q-93; P-17 to G-92; P-17 to T-91; P-17 to P-90; P-17 to V-89; P-17 to C-88; P-17 to E-87; P-17 to L-86; P-17 to G-85; P-17 to D-84; P-17 to D-83; P-17 to P-82; of SEQ ID NO:2. Preferably, any of the above listed N- or C-terminal deletions can be combined to produce a N- and C-terminal deleted VEGF-3 polypeptide, which retains the conserved region.

Moreover, the invention also provides polypeptides having one or more amino acids deleted from both the amino and the carboxyl termini, which may be described generally as having residues m-n of SEQ ID NO:2, where n and m are integers as described above.

Many polynucleotide sequences, such as EST sequences, are publicly available and accessible through sequence databases. Some of these sequences are related to SEQ ID NO:1 or SEQ ID NO:19 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 655 of SEQ ID NO:1, b is an integer of 15 to 666, where both a and b correspond to the positions of nucleotide residues shown in SEQ ID NO:1, and where the b is greater than or equal to a +14. Also preferably excluded from the present invention are one or more polynucleotides comprising a nucleotide sequence described by the general formula of c−d, where c is any integer between 1 to 610 of SEQ ID NO:20, d is an integer of 15 to 618, where both c and d correspond to the positions of nucleotide residues shown in SEQ ID NO:19, and where d is greater than or equal to c+14.

For example, the following sequences are related to SEQ ID NOs: 1 and 19, GenBank Accession Nos.: AA434485 (SEQ ID NO:21); AA292448 (SEQ ID NO:22); AA310070 (SEQ ID NO:23); AA073660 (SEQ ID NO:24); H39505 (SEQ ID NO:25); R90829 (SEQ ID NO:26); AA082818 (SEQ ID NO:27); AA117672 (SEQ ID NO:28); AA040843 (SEQ ID NO:29); T08411 (SEQ ID NO:30); AA419103 (SEQ ID NO:31); AA633535 (SEQ ID NO:32); AA182397 (SEQ ID NO:33); AA843665 (SEQ ID NO:34); AA434389 (SEQ ID NO:35); AA073557 (SEQ ID NO:36); AA741539 (SEQ ID NO:37); R88630 (SEQ ID NO:38); AA843530 (SEQ ID NO:39); AA510867 (SEQ ID NO:40); N87395 (SEQ ID NO:41); AA252383 (SEQ ID NO:42); AA284431 (SEQ ID NO:43); AA252749 (SEQ ID NO:44); AA259024 (SEQ ID NO:45); AA612827 (SEQ ID NO:46); AA163579 (SEQ ID NO:47); AA290917 (SEQ ID NO:48); H27946 (SEQ ID NO:49); AA801448 (SEQ ID NO:50); AA236770 (SEQ ID NO:51); AA799651 (SEQ ID NO:52); AA891731 (SEQ IDNO:53); AA893520 (SEQ ID NO:54); AA800195 (SEQ ID NO:55); AA568606 (SEQ ID NO:56); AA465305 (SEQ ID NO:57); AA420778 (SEQ ID NO:58); AA308077 (SEQ ID NO:59); AA484390 (SEQ ID NO:60); AA633294 (SEQ ID NO:61); AA252684 (SEQ ID NO:62); AA640496 (SEQ ID NO:63); AA535586 (SEQ ID NO:64); AA491141 (SEQ ID NO:65); AA663755 (SEQ ID NO:66); W75461 (SEQ ID NO:67); AA252835 (SEQ ID NO:68); AA240250 (SEQ ID NO:69); AA050707 (SEQ ID NO:70); AA239623 (SEQ IfDNO:71); W46077 (SEQ ID NO:72); W91423 (SEQ ID NO:73); AA239292 (SEQ ID NO:74); AA511126 (SEQ ID NO:75); AA220465 (SEQ ID NO:76); AA024319 (SEQ ID NO:77); AA703302 (SEQ ID NO:78); AA776045 (SEQ ID NO:79); AA481592 (SEQ ID NO:80); AA239633 (SEQ ID NO:81); AA809010 (SEQ ID NO:82); W50682 (SEQ ID NO:83); W71063 (SEQ ID NO:84); W53314 (SEQ ID NO:85); W10329 (SEQ ID NO:86); AA221061 (SEQ ID NO:87); AA799590 (SEQ ID NO:88); AA289037 (SEQ ID NO:89); AA161245 (SEQ ID NO:90); AA799406 (SEQ ID NO:91); T27013 (SEQ ID NO:92); AA492515 (SEQ ID NO:93); AA654668 (SEQ ID NO:94); AA848532 (SEQ ID NO:95); AA849744 (SEQ ID NO:96); AA943751 (SEQ ID NO:97); AA412813 (SEQ ID NO:98); AA632887 (SEQ ID NO:99); AA944311 (SEQ ID NO:100); AA568336 (SEQ ID NO:101); AA579388 (SEQ ID NO:102); AA873930 (SEQ ID NO:103); AI019996 (SEQ ID NO:104); W70867 (SEQ ID NO:105); AA524977 (SEQ ID NO:106); AA491065 (SEQ ID NO:107); M78808 (SEQ ID NO:108); AA822530 (SEQ ID NO:109); AA869378 (SEQ IDNO:110); AA420802 (SEQ ID NO:111); AA280249 (SEQ ID NO:112); AA287979 (SEQ ID NO:113); AA499558 (SEQ ID NO:114); H11172 (SEQ ID NO:115); R19956 (SEQ ID NO:116); AA350839 (SEQ ID NO:117); AA559311 (SEQ ID NO:118); AA578206 (SEQ ID NO:119); AI040483 (SEQ ID NO:120); AA283649 (SEQ ID NO:121); AA359891 (SEQ ID NO:122); AA559170 (SEQ ID NO:123); AA359370 (SEQ ID NO:124); R71959 (SEQ ID NO:125); AA612297 (SEQ IDNO:126); AA657654 (SEQ ID NO:127); W53375 (SEQ ID NO:128); AA998090 (SEQ ID NO:129); AA284028 (SEQ ID NO:130); AA560000 (SEQ ID NO:131); AA014203 (SEQ ID NO:132); AA689410 (SEQ ID NO:133); AA215511 (SEQ ID NO:134); AA284432 (SEQ ID NO:135); AA573045 (SEQ ID NO:136); AA956224 (SEQ ID NO:137); and AA998751 (SEQ ID NO:138).

The following sequences are related to SEQ ID NO:19, GenBank Accession Nos.: AI174183 (SEQ ID NO:139); AI155033 (SEQ ID NO:140); AI117413 (SEQIDNO:141); AI141331 (SEQ ID NO:142);AA917955 (SEQ ID NO:143); AI186160 (SEQ ID NO:144); AI009149 (SEQ ID NO:145); AA946047 (SEQ ID NO:146); AI008710 (SEQ ID NO:147); AI008706 (SEQ ID NO:148); AA944521 (SEQ ID NO:149); AA221493 (SEQ ID NO:150); AA267131 (SEQ ID NO:151); AI125118 (SEQ ID NO:152); and AI129752 (SEQ ID NO:153).

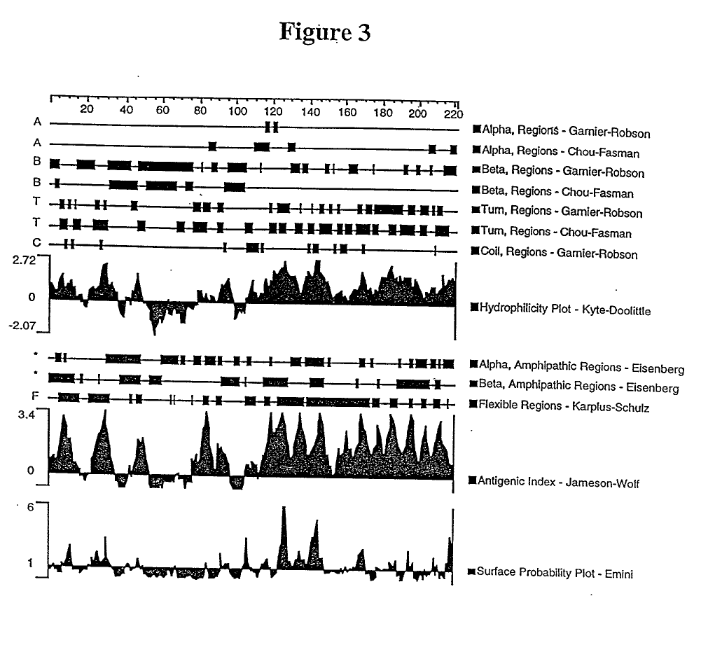

Also preferred are VEGF-3 polypeptide and polynucleotide fragments characterized by structural or functional domains. Preferred embodiments of the invention include fragments that comprise alpha-helix and alpha-helix forming regions (“alpha-regions”), beta-sheet and beta-sheet-forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, flexible regions, surface-forming regions, substrate binding region, and high antigenic index regions. As set out in the Figures, such preferred regions include Garnier-Rob son alpha-regions, beta-regions, turn-regions, and coil-regions, Chou-Fasman alpha-regions, beta-regions, and turn-regions, Kyte-Doolittle hydrophilic regions and hydrophobic regions, Eisenberg alpha and beta amphipathic regions, Karplus-Schulz flexibleregions, Emini surface-forming regions, and Jameson-Wolf high antigenic index regions. Polypeptide fragments of SEQ ID NO:2 falling within conserved domains are specifically contemplated by the present invention. (See FIG. 3.) Moreover, polynucleotide fragments encoding these domains are also contemplated.

Other preferred fragments are biologically active VEGF-3 fragments. Biologically active fragments are those exhibiting activity similar, but not necessarily identical, to an activity of the VEGF-3 polypeptide. The biological activity of the fragments may include an improved desired activity, or a decreased undesirable activity.

Epitopes & Antibodies

In the present invention, “epitopes” refer to VEGF-3 polypeptide fragments having antigenic or immunogenic activity in an animal, especially in a human. A preferred embodiment of the present invention relates to a VEGF-3 polypeptide fragment comprising an epitope, as well as the polynucleotide encoding this fragment. A region of a protein molecule to which an antibody can bind is defined as an “antigenic epitope.” In contrast, an “immunogenic epitope” is defined as a part of a protein that elicits an antibody response. (See, for instance, Geysen et al, Proc. Natl. Acad. Sci. USA 81:3998-4002 (1983).) Fragments which function as epitopes may be produced by any conventional means. (See, e.g., Houghten, R. A., Proc. Natl. Acad. Sci. USA 82:5131-5135 (1985) further described in U.S. Pat. No. 4,631,211.).

In the present invention, antigenic epitopes preferably contain a sequence of at least seven, more preferably at least nine, and most preferably between about 15 to about 30 amino acids. Antigenic epitopes are useful to raise antibodies, including monoclonal antibodies, that specifically bind the epitope. (See, for instance, Wilson et al., Cell 37:767-778 (1984); Sutcliffe, J. G. et al., Science 219:660-666 (1983).)

Similarly, immunogenic epitopes can be used to induce antibodies according to methods well known in the art. (See, for instance, Sutcliffe et al., supra; Wilson et al., supra; Chow, M. et al, Proc. Natl. Acad. Sci. USA 82:910-914; and Bittle, F. J. et al, J. Gen. Virol. 66:2347-2354 (1985).) A preferred immunogenic epitope includes the secreted protein. The immunogenic epitopes may be presented together with a carrier protein, such as an albumin, to an animal system (such as rabbit or mouse) or, if it is long enough (at least about 25 amino acids), without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting.).

Using DNAstar analysis, SEQ ID NO:2 was found antigenic at amino acids: Q24-V34; T45-V52; C77-V89; V89-Q95; S114-A131; A131-T139; T139-S154; A155-1164; T165-C175; P176-H182; H182-C192; C192-R201; R201-E208; and E208-R220. Thus, these regions could be used as epitopes to produce antibodies against the protein encoded by HMWCF06 .

Using DNAstar analysis, SEQ ID NO:20 was found to be antigenic at amino acids: Q24-V34; T45-V52; C77-V89; S114-A131; A131-T139; T139-S154; and P156 to D163. Thus, these regions could be used as epitopes to produce antibodies against the protein encoded by HMWCF06.

As used herein, the term “antibody” (Ab) or “monoclonal antibody” (Mab) is meant to include intact molecules as well as antibody fragments (such as, for example, Fab and F(ab′)2 fragments) which are capable of specifically binding to protein. Fab and F(ab′)2 fragments lack the Fc fragment of intact antibody, clear more rapidly from the circulation, and may have less non-specific tissue binding than an intact antibody. (Wahl et al, J. Nucl. Med. 24:316-325 (1983).) Thus, these fragments are preferred, as well as the products of a Fab or other immunoglobulin expression library. Moreover, antibodies of the present invention include chimeric, single chain, and humanized antibodies.

Fusion Proteins

Any VEGF-3 polypeptide can be used to generate fusion proteins. For example, the VEGF-3 polypeptide, when fused to a second protein, can be used as an antigenic tag. Antibodies raised against the VEGF-3 polypeptide can be used to indirectly detect the second protein by binding to the VEGF-3. Moreover, because secreted proteins target cellular locations based on trafficking signals, the VEGF-3 polypeptides can be used as a targeting molecule once fused to other proteins.

Examples of domains that can be fused to VEGF-3 polypeptides include not only heterologous signal sequences, but also other heterologous functional regions. The fusion does not necessarily need to be direct, but may occur through linker sequences.

In certain preferred embodiments, VEGF-3 fusion polypeptides may be constructed which include additional N-terminal and/or C-terminal amino acid residues. In particular, any N-terminally or C-terminally deleted VEGF-3 polypeptide disclosed in the successive lists provided above may be altered by inclusion of additional amino acid residues at the N-terminus to produce a VEGF-3 fusion polypeptide. In addition, VEGF-3 fusion polypeptides are contemplated which include additional N-terminal and/or C-terminal amino acid residues fused to a VEGF-3 polypeptide comprising any combination of—and C-terminal deletions set forth in the lists provided above.

In additional preferred embodiments, specific amino acid residues which are contemplated to be fused to the N-terminus of any N-terminally or C-terminally deleted VEGF-3 polypeptide disclosed in the successive lists provided above include the following amino acid sequences: MSPLLRRLLLAALLQLA (SEQ ID NO:154); MSPLLRRLLLAALLQL (SEQ IID NO:155); MSPLLRRLLLAALLQ (SEQ ID NO:156); MSPLLRRLLLAALL (SEQ ID NO:157); MSPLLRRLLLAAL (SEQ ID NO:158); MSPLLRRLLLAA (SEQ ID NO:159); MSPLLRRLLLA (SEQ ID NO:160); MSPLLRRLLL (SEQ ID NO:161); MSPLLRRLL (SEQ ID NO:162); MSPLLRRL (SEQ ID NO:163); MSPLLRR (SEQ ID NO:164); MSPLLR (SEQ ID NO:165); MSPLL (SEQ ID NO:166); MSPL (SEQ ID NO:167); MSP; MS; M; SPLLRRLLLAALLQLA (SEQ ID NO:168); MSPLLRRLLLAALLQLA (SEQ ID NO:169); MPLLRRLLLAALLQLA (SEQ ID NO:170); PLLRRLLLAALLQLA (SEQ ID NO:171); MLLRRLLLAALLQLA (SEQ ID NO:172); LLRRLLLAALLQLA (SEQ ID NO:173); MLRRLLLAALLQLA (SEQ ID NO:174); LRRLLLAALLQLA (SEQ ID NO:175); MRRLLLAALLQLA (SEQ ID NO:176); RRLLLAALLQLA (SEQ ID NO:177); MRLLLAALLQLA (SEQ ID NO:178); RLLLAALLQLA (SEQ ID NO:179); MLLLAALLQLA (SEQ ID NO:180); LLLAALLQLA (SEQ ID NO:181); MLLAALLQLA (SEQ ID NO:182); LLAALLQLA (SEQ ID NO:183); MLAALLQLA (SEQ ID NO:184); LAALLQLA (SEQ ID NO:185); MAALLQLA (SEQ ID NO:186); AALLQLA (SEQ ID NO:187); MALLQLA (SEQ ID NO:188); ALLQLA (SEQ ID NO:189); MLLQLA (SEQ ID NO:190); LLQLA (SEQ ID NO:191); MLQLA (SEQ ID NO:192); LQLA (SEQ ID NO:193); MQLA (SEQ ID NO:194); QLA; MLA; LA; MA or A. Polynucleotides encoding these fusion polypeptides are also provided. Moreover, it is also contemplated that protein may be expressed from any of the muteins and/or fusion polypeptides described above (for a non-limiting exemplary expression protocol, see Example 5).

Moreover, fusion proteins may also be engineered to improve characteristics of the VEGF-3 polypeptide. For instance, a region of additional amino acids, particularly charged amino acids, may be added to the N-terminus of the VEGF-3 polypeptide to improve stability and persistence during purification from the host cell or subsequent handling and storage. Also, peptide moieties may be added to the VEGF-3 polypeptide to facilitate purification. Such regions may be removed prior to final preparation of the VEGF-3 polypeptide. The addition of peptide moieties to facilitate handling of polypeptides are familiar and routine techniques in the art.

Moreover, VEGF-3 polypeptides, including fragments, and specifically epitopes, can be combined with parts of the constant domain of immunoglobulins (IgG), resulting in chimeric polypeptides. These fusion proteins facilitate purification and show an increased half-life in vivo. One reported example describes chimeric proteins consisting of the first two domains of the human CD4-polypeptide and various domains of the constant regions of the heavy or light chains of mammalian immunoglobulins. (EP A 394,827; Traunecker et al., Nature 331:84-86 (1988).) Fusion proteins having disulfide-linked dimeric structures (due to the IgG) can also be more efficient in binding and neutralizing other molecules, than the monomeric secreted protein or protein fragment alone. (Fountoulakis et al., J. Biochem. 270:3958-3964 (1995).).

Similarly, EP-A-O 464 533 (Canadian counterpart 2045869) discloses fusion proteins comprising various portions of constant region of immunoglobulin molecules together with another human protein or part thereof In many cases, the Fc part in a fusion protein is beneficial in therapy and diagnosis, and thus can result in, for example, improved pharmacokinetic properties. (EP-A 0232 262.) Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5. (See, D. Bennett et al., J. Molecular Recognition 8:52-58 (1995); K. Johanson et al., J. Biol. Chem. 270:9459-9471 (1995).).

Moreover, the VEGF-3 polypeptides can be fused to marker sequences, such as a peptide which facilitates purification of VEGF-3. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Another peptide tag useful for purification, the “HA” tag, corresponds to an epitope derived from the influenza hemagglutinin protein. (Wilson et al., Cell 37:767 (1984).).

Thus, any of these above fusions can be engineered using the VEGF-3 polynucleotides or the polypeptides.

Vectors, Host Cells, and Protein Production

The present invention also relates to vectors containing the VEGF-3 polynucleotide, host cells, and the production of polypeptides by recombinant techniques. The vector may be, for example, a phage, plasmid, viral, or retroviral vector. Retroviral vectors may be replication competent or replication defective. In the latter case, viral propagation generally will occur only in complementing host cells.

VEGF-3 polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The VEGF-3 polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293, and Bowes melanoma cells; and plant cells. Appropriate culture mediums and conditions for the above-described host cells are known in the art.

Among vectors preferred for use in bacteria include pHE-4 (and variants thereof); pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that VEGF-3 polypeptides may in fact be expressed by a host cell lacking a recombinant vector.

VEGF-3 polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

VEGF-3 polypeptides, and preferably the secreted form, can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant productionprocedure, the VEGF-3 polypeptides maybe glycosylated or may be non-glycosylated. In addition, VEGF-3 polypeptides may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g. VEGF-3 coding sequence), and/or to include genetic material (e.g., heterologous polynucleotide sequences) that is operably associated with VEGF-3 polynucleotides of the invention, and which activates, alters, and/or amplifies endogenous VEGF-3 polynucleotides. For example, techniques known in the art may be used to operably associate heterologous control regions (e.g., promoter and/or enhancer) and endogenous VEGF-3 polynucleotide sequences via homologous recombination (see, e.g., U.S. Pat. No. 5,641,670, issued Jun. 24, 1997; International Publication No. WO 96/29411, published Sep. 26,1996; International Publication No. WO 94/12650, published Aug. 4, 1994; Koller et al., Proc. Natl. Acad. Sci. USA 86:8932-8935 (1989); and Zijlstra et al., Nature 342:435-438 (1989), the disclosures of each of which are incorporated by reference in their entireties).

Uses of the VEGF-3 Polynucleotides

The VEGF-3 polynucleotides identified herein can be used in numerous ways as reagents. The following description should be considered exemplary and utilizes known techniques.

There exists an ongoing need to identify new chromosome markers, since few chromosome marking reagents, based on actual sequence data (repeat polymorphisms), are presently available.

Briefly, sequences can be mapped to chromosomes by preparing PCR primers (preferably 15-25 bp) from the sequences shown in SEQ ID NO:1 or SEQ ID NO:19. Primers can be selected using computer analysis so that primers do not span more than one predicted exon in the genomic DNA. These primers are then used for PCR screening of somatic cell hybrids containing individual human chromosomes. Only those hybrids containing the human VEGF-3 gene corresponding to the SEQ ID NO:1 or SEQ ID NO:19 will yield an amplified fragment.

Similarly, somatic hybrids provide a rapid method of PCR mapping the polynucleotides to particular chromosomes. Three or more clones can be assigned per day using a single thermal cycler. Moreover, sublocalization of the VEGF-3 polynucleotides can be achieved with panels of specific chromosome fragments. Other gene mapping strategies that can be used include in situ hybridization, prescreening with labeled flow-sorted chromosomes, and preselection by hybridization to construct chromosome specific-cDNA libraries.

Precise chromosomal location of the VEGF-3 polynucleotides can also be achieved using fluorescence in situ hybridization (FISH) of a metaphase chromosomal spread. This technique uses polynucleotides as short as 500 or 600 bases; however, polynucleotides 2,000-4,000 bp are preferred. For a review of this technique, see Verma et al., “Human Chromosomes: a Manual of Basic Techniques,” Pergamon Press, New York (1988).

For chromosome mapping, the VEGF-3 polynucleotides can be used individually (to mark a single chromosome or a single site on that chromosome) or in panels (for marking multiple sites and/or multiple chromosomes). Preferred polynucleotides correspond to the noncoding regions of the cDNAs because the coding sequences are more likely conserved within gene families, thus increasing the chance of cross hybridization during chromosomal mapping.

Once a polynucleotide has been mapped to a precise chromosomal location, the physical position of the polynucleotide can be used in linkage analysis. Linkage analysis establishes coinheritance between a chromosomal location and presentation of a particular disease. (Disease mapping data are found, for example, in V. McKusick, Mendelian Inheritance in Man (available on line through Johns Hopkins University Welch Medical Library).) Assuming 1 megabase mapping resolution and one gene per 20 kb, a cDNA precisely localized to a chromosomal region associated with the disease could be one of 50-500 potential causative genes.

Thus, once coinheritance is established, differences in the VEGF-3 polynucleotide and the corresponding gene between affected and unaffected individuals can be examined. First, visible structural alterations in the chromosomes, such as deletions or translocations, are examined in chromosome spreads or by PCR. If no structural alterations exist, the presence of point mutations are ascertained. Mutations observed in some or all affected individuals, but not in normal individuals, indicates that the mutation may cause the disease. However, complete sequencing of the VEGF-3 polypeptide and the corresponding gene from several normal individuals is required to distinguish the mutation from a polymorphism. If a new polymorphism is identified, this polymorphic polypeptide can be used for further linkage analysis.

Furthermore, increased or decreased expression of the gene in affected individuals as compared to unaffected individuals can be assessed using VEGF-3 polynucleotides. Any of these alterations (altered expression, chromosomal rearrangement, or mutation) can be used as a diagnostic or prognostic marker.

In addition to the foregoing, a VEGF-3 polynucleotide can be used to control gene expression through triple helix formation or antisense DNA or RNA. Both methods rely on binding of the polynucleotide to DNA or RNA. For these techniques, preferred polynucleotides are usually 20 to 40 bases in length and complementary to either the region of the gene involved in transcription (triple helix—see Lee et al., Nucl. Acids Res. 6:3073 (1979); Cooney et al., Science 241:456 (1988); and Dervan et al., Science 251:1360 (1991) ) or to the mRNA itself (antisense—Okano, J. Neurochem. 56:560 (1991); Oligodeoxy-nucleotides as Antisense Inhibitors of Gene Expression, CRC Press, Boca Raton, Fla. (1988).) Triple helix formation optimally results in a shut-off of RNA transcription from DNA, while antisense RNA hybridization blocks translation of an mRNA molecule into polypeptide. Both techniques are effective in model systems, and the information disclosed herein can be used to design antisense or triple helix polynucleotides in an effort to treat disease.

VEGF-3 polynucleotides are also useful in gene therapy. One goal of gene therapy is to insert a normal gene into an organism having a defective gene, in an effort to correct the genetic defect. VEGF-3 offers a means of targeting such genetic defects in a highly accurate manner. Another goal is to insert a new gene that was not present in the host genome, thereby producing a new trait in the host cell.

The VEGF-3 polynucleotides are also useful for identifying individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identifying personnel. This method does not suffer from the current limitations of “Dog Tags” which can be lost, switched, or stolen, making positive identification difficult. The VEGF-3 polynucleotides can be used as additional DNA markers for RFLP.

The VEGF-3 polynucleotides can also be used as an alternative to RFLP, by determining the actual base-by-base DNA sequence of selected portions of an individual's genome. These sequences can be used to prepare PCR primers for amplifying and isolating such selected DNA, which can then be sequenced. Using this technique, individuals can be identified because each individual will have a unique set of DNA sequences. Once an unique ID database is established for an individual, positive identification of that individual, living or dead, can be made from extremely small tissue samples.

Forensic biology also benefits from using DNA-based identification techniques as disclosed herein. DNA sequences taken from very small biological samples such as tissues, e.g., hair or skin, or body fluids, e.g., blood, saliva, semen, etc., can be amplified using PCR. In one prior art technique, gene sequences amplified from polymorphic loci, such as DQa class II HLA gene, are used in forensic biology to identify individuals. (Erlich, H., PCR Technology, Freeman and Co. (1992).) Once these specific polymorphic loci are amplified, they are digested with one or more restriction enzymes, yielding an identifying set of bands on a Southern blot probed with DNA corresponding to the DQa class II HLA gene. Similarly, VEGF-3 polynucleotides can be used as polymorphic markers for forensic purposes.

There is also a need for reagents capable of identifying the source of a particular tissue. Such need arises, for example, in forensics when presented with tissue of unknown origin. Appropriate reagents can comprise, for example, DNA probes or primers specific to particular tissue prepared from VEGF-3 sequences. Panels of such reagents can identify tissue by species and/or by organ type. In a similar fashion, these reagents can be used to screen tissue cultures for contamination.

Because VEGF-3 is found expressed in colon, heart, kidney, and ovary, VEGF-3 polynucleotides are useful as hybridization probes for differential identification of the tissue(s) or cell type(s) present in a biological sample. Similarly, polypeptides and antibodies directed to VEGF-3 polypeptides are useful to provide immunological probes for differential identification of the tissue(s) or cell type(s). In addition, for a number of disorders of the above tissues or cells, particularly of the vascular and lymphatic system, significantly higher or lower levels of VEGF-3 gene expression may be detected in certain tissues (e.g., cancerous and wounded tissues) or bodily fluids (e.g., serum, plasma, urine, synovial fluid or spinal fluid) taken from an individual having such a disorder, relative to a “standard” VEGF-3 gene expression level, i.e., the VEGF-3 expression level in healthy tissue from an individual not having the vascular and lymphatic system disorder.

Thus, the invention provides a diagnostic method of a disorder, which involves: (a) assaying VEGF-3 gene expression level in cells or body fluid of an individual; (b) comparing the VEGF-3 gene expression level with a standard VEGF-3 gene expression level, whereby an increase or decrease in the assayed VEGF-3 gene expression level compared to the standard expression level is indicative of disorder in the vascular and lymphatic system.

In the very least, the VEGF-3 polynucleotides can be used as molecular weight markers on Southern gels, as diagnostic probes for the presence of a specific MRNA in a particular cell type, as a probe to “subtract-out” known sequences in the process of discovering novel polynucleotides, for selecting and making oligomers for attachment to a “gene chip” or other support, to raise anti-DNA antibodies using DNA immunization techniques, and as an antigen to elicit an immune response.

Uses of VEGF-3 Polypeptides

VEGF-3 polypeptides can be used in numerous ways. The following description should be considered exemplary and utilizes known techniques.

VEGF-3 polypeptides can be used to assay protein levels in a biological sample using antibody-based techniques. For example, protein expression in tissues can be studied with classical immunohistological methods. (Jalkanen, M., et al., J. Cell. Biol. 101:976-985 (1985); Jalkanen, M. et al., J. Cell. Biol. 105:3087-3096 (1987).) Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (¹²⁵I, ¹¹²I), carbon (¹⁴C), sulfur (³⁵ S), tritium (³H), indium (¹¹²In), and technetium (⁹⁹mTc), and fluorescent labels, such as fluorescein and rhodamine, and biotin.

In addition to assaying secreted protein levels in a biological sample, proteins can also be detected in vivo by imaging. Antibody labels or markers for in vivo imaging of protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A protein-specific antibody or antibody fragment which has been labeled with an appropriate detectable imaging moiety, such as a radioisotope (for example, ¹³¹I, ¹¹²In, ⁹⁹mTc), a radio-opaque substance, or a material detectable by nuclear magnetic resonance, is introduced (for example, parenterally, subcutaneously, or intraperitoneally) into the mammal. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99mTc. The labeled antibody or antibody fragment will then preferentially accumulate at the location of cells which contain the specific protein. In vivo tumor imaging is described in S.W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).).

Thus, the invention provides a diagnostic method of a disorder, which involves (a) assaying the expression of VEGF-3 polypeptide in cells or body fluid of an individual; (b) comparing the level of gene expression with a standard gene expression level, whereby an increase or decrease in the assayed VEGF-3 polypeptide gene expression level compared to the standard expression level is indicative of a disorder.

Moreover, VEGF-3 polypeptides can be used to treat disease. For example, patients can be administered VEGF-3 polypeptides in an effort to replace absent or decreased levels of the VEGF-3 polypeptide (e.g., insulin), to supplement absent or decreased levels of a different polypeptide (e.g., hemoglobin S for hemoglobin B), to inhibit the activity of a polypeptide (e.g., an oncogene), to activate the activity of a polypeptide (e.g., by binding to a receptor), to reduce the activity of a membrane bound receptor by competing with it for free ligand (e.g., soluble TNF receptors used in reducing inflammation), or to bring about a desired response (e.g., blood vessel growth).

Similarly, antibodies directed to VEGF-3 polypeptides can also be used to treat disease. For example, administration of an antibody directed to a VEGF-3 polypeptide can bind and reduce overproduction of the polypeptide. Similarly, administration of an antibody can activate the polypeptide, such as by binding to a polypeptide bound to a membrane (receptor).

At the very least, the VEGF-3 polypeptides can be used as molecular weight markers on SDS-PAGE gels or on molecular sieve gel filtration columns using methods well known to those of skill in the art. VEGF-3 polypeptides can also be used to raise antibodies, which in turn are used to measure protein expression from a recombinant cell, as a way of assessing transformation of the host cell. Moreover, VEGF-3 polypeptides can be used to test the following biological activities.

Biological Activities of VEGF-3

VEGF-3 polynucleotides and polypeptides can be used in assays to test for one or more biological activities. If VEGF-3 polynucleotides and polypeptides do exhibit activity in a particular assay, it is likely that VEGF-3 may be involved in the diseases associated with the biological activity. Therefore, VEGF-3 could be used to treat the associated disease.

Immune Activity

VEGF-3 polypeptides or polynucleotides may be useful in treating deficiencies or disorders of the immune system, by activating or inhibiting the proliferation, differentiation, or mobilization (chemotaxis) of immune cells. Immune cells develop through a process called hematopoiesis, producing myeloid (platelets, red blood cells, neutrophils, and macrophages) and lymphoid (B and T lymphocytes) cells from pluripotent stem cells. The etiology of these immune deficiencies or disorders may be genetic, somatic, such as cancer or some autoimmune disorders, acquired (e.g., by chemotherapy or toxins), or infectious. Moreover, VEGF-3 polynucleotides or polypeptides can be used as a marker or detector of a particular immune system disease or disorder.

VEGF-3 polynucleotides or polypeptides may be useful in treating or detecting deficiencies or disorders of hematopoietic cells. VEGF-3 polypeptides or polynucleotides could be used to increase differentiation and proliferation of hematopoietic cells, including the pluripotent stem cells, in an effort to treat those disorders associated with a decrease in certain (or many) types hematopoietic cells. Examples ofimmunologic deficiency syndromes include, but are not limited to: blood protein disorders (e.g. agammaglobulinemia, dysgammaglobulinemia), ataxia telangiectasia, common variable immunodeficiency, Digeorge Syndrome, HIV infection, HTLV-BLV infection, leukocyte adhesion deficiency syndrome, lymphopenia, phagocyte bactericidal dysfunction, severe combined immunodeficiency (SCIDs), Wiskott-Aldrich Disorder, anemia, thrombocytopenia, or hemoglobinuria.

Moreover, VEGF-3 polypeptides or polynucleotides can also be used to modulate hemostatic (the stopping of bleeding) or thrombolytic activity (clot formation). For example, by increasing hemostatic or thrombolytic activity, VEGF-3 polynucleotides or polypeptides could be used to treat blood coagulation disorders (e.g., afibrinogenemia, factor deficiencies), blood platelet disorders (e.g. thrombocytopenia), or wounds resulting from trauma, surgery, or other causes. Alternatively, VEGF-3 polynucleotides or polypeptides that can decrease hemostatic or thrombolytic activity could be used to inhibit or dissolve clotting, important in the treatment of heart attacks (infarction), strokes, or scarring.

VEGF-3 polynucleotides or polypeptides may also be useful in treating or detecting autoimmune disorders. Many autoimmune disorders result from inappropriate recognition of self as foreign material by immune cells. This inappropriate recognition results in an immune response leading to the destruction of the host tissue. Therefore, the administration of VEGF-3 polypeptides or polynucleotides that can inhibit an immune response, particularly the proliferation, differentiation, or chemotaxis of T-cells, may be an effective therapy in preventing autoimmune disorders.

Examples of autoimmune disorders that can be treated or detected by VEGF-3 include, but are not limited to: Addison's Disease, hemolytic anemia, antiphospholipid syndrome, rheumatoid arthritis, dermatitis, allergic encephalomyelitis, glomerulonephritis, Goodpasture's Syndrome, Graves' Disease, Multiple Sclerosis, Myasthenia Gravis, Neuritis, Ophthalmia, Bullous Pemphigoid, Pemphigus, Polyendocrinopathies, Purpura, Reiter's Disease, Stiff-Man Syndrome, Autoimmune Thyroiditis, Systemic Lupus Erythematosus, Autoimmune Pulmonary Inflammation, Guillain-Barre Syndrome, insulin dependent diabetes mellitis, and autoimmune inflammatory eye disease.

Similarly, allergic reactions and conditions, such as asthma (particularly allergic asthma) or other respiratory problems, may also be treated by VEGF-3 polypeptides or polynucleotides. Moreover, VEGF-3 can be used to treat anaphylaxis, hypersensitivity to an antigenic molecule, or blood group incompatibility.

VEGF-3 polynucleotides or polypeptides may also be used to treat and/or prevent organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration ofVEGF-3 polypeptides or polynucleotides that inhibits an immune response, particularly the proliferation, differentiation, or chemotaxis of T-cells, may be an effective therapy in preventing organ rejection or GVHD.

Similarly, VEGF-3 polypeptides or polynucleotides may also be used to modulate inflammation. For example, VEGF-3 polypeptides or polynucleotides may inhibit the proliferation and differentiation of cells involved in an inflammatory response. These molecules can be used to treat inflammatory conditions, both chronic and acute conditions, including inflammation associated with infection (e.g., septic shock, sepsis, or systemic inflammatory response syndrome (SIRS)), ischemia-reperfusion injury, endotoxin lethality, arthritis, complement-mediated hyperacute rejection, nephritis, cytokine or chemokine induced lung injury, inflammatory bowel disease, Crohn's disease, or resulting from over production of cytokines (e.g., TNF or IL-1.)

Hyperproliferative Disorders

VEGF-3 polypeptides or polynucleotides can be used to treat or detect hyperproliferative disorders, including neoplasms. VEGF-3 polypeptides or polynucleotides may inhibit the proliferation of the disorder through direct or indirect interactions. Alternatively, VEGF-3 polypeptides or polynucleotides may proliferate other cells which can inhibit the hyperproliferative disorder.

For example, by increasing an immune response, particularly increasing antigenic qualities of the hyperproliferative disorder or by proliferating, differentiating, or mobilizing T-cells, hyperproliferative disorders can be treated. This immune response may be increased by either enhancing an existing immune response, or by initiating a new immune response. Alternatively, decreasing an immune response may also be a method of treating hyperproliferative disorders, such as a chemotherapeutic agent.

Examples of hyperproliferative disorders that can be treated or detected by VEGF-3 polynucleotides or polypeptides include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital.

Similarly, other hyperproliferative disorders can also be treated or detected by VEGF-3 polynucleotides or polypeptides. Examples of such hyperproliferative disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

Infectious Disease

VEGF-3 polypeptides or polynucleotides can be used to treat or detect infectious agents. For example, by increasing the immune response, particularly increasing the proliferation and differentiation of B and/or T cells, infectious diseases may be treated. The immune response may be increased by either enhancing an existing immune response, or by initiating a new immune response. Alternatively, VEGF-3 polypeptides or polynucleotides may also directly inhibit the infectious agent, without necessarily eliciting an immune response.

Viruses are one example of an infectious agent that can cause disease or symptoms that can be treated or detected by VEGF-3 polynucleotides or polypeptides. Examples of viruses, include, but are not limited to the following DNA and RNA viral families: Arbovirus, Adenoviridae, Arenaviridae, Arterivirus, Birnaviridae, Bunyaviridae, Caliciviridae, Circoviridae, Coronaviridae, Flaviviridae, Hepadnaviridae (Hepatitis), Herpesviridae (such as, Cytomegalovirus, Herpes Simplex, Herpes Zoster), Mononegavirus (e.g., Paramyxoviridae, Morbillivirus, Rhabdoviridae), Orthomyxoviridae (e.g., Influenza), Papovaviridae, Parvoviridae, Picomaviridae, Poxviridae (such as Smallpox or Vaccinia), Reoviridae (e.g., Rotavirus), Retroviridae (HTLV-I, HTLV-II, Lentivirus), and Togaviridae (e.g., Rubivirus). Viruses falling within these families can cause a variety of diseases or symptoms, including, but not limited to: arthritis, bronchiollitis, encephalitis, eye infections (e.g., conjunctivitis, keratitis), chronic fatigue syndrome, hepatitis (A, B, C, E, Chronic Active, Delta), meningitis, opportunistic infections (e.g., AIDS), pneumonia, Burkitt's Lymphoma, chickenpox, hemorrhagic fever, Measles, Mumps, Parainfluenza, Rabies, the common cold, Polio, leukemia, Rubella, sexually transmitted diseases, skin diseases (e.g., Kaposi's, warts), and viremia. VEGF-3 polypeptides or polynucleotides can be used to treat or detect any of these symptoms or diseases.

Similarly, bacterial or fungal agents that can cause disease or symptoms and that can be treated or detected by VEGF-3 polynucleotides or polypeptides include, but not limited to, the following Gram-Negative and Gram-positive bacterial families and fungi: Actinomycetales (e.g., Corynebacterium, Mycobacterium, Norcardia), Aspergillosis, Bacillaceae (e.g., Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, Legionellosis, Leptospirosis, Listeria, Mycoplasmatales, Neisseriaceae (e.g., Acinetobacter, Gonorrhea, Menigococcal), Pasteurellacea Infections (e.g., Actinobacillus, Heamophilus, Pasteurella), Pseudomonas, Rickettsiaceae, Chlamydiaceae, Syphilis, and Staphylococcal. These bacterial or fungal families can cause the following diseases or symptoms, including, but not limited to: bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping Cough or Empyema, sepsis, Lyme Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, wound infections. VEGF-3 polypeptides or polynucleotides can be used to treat or detect any of these symptoms or diseases.

Moreover, parasitic agents causing disease or symptoms that can be treated or detected by VEGF-3 polynucleotides or polypeptides include, but not limited to, the following families: Amebiasis, Babesiosis, Coccidiosis, Cryptosporidiosis, Dientamoebiasis, Dourine, Ectoparasitic, Giardiasis, Helminthiasis, Leishmaniasis, Theileriasis, Toxoplasmosis, Trypanosomiasis, and Trichomonas. These parasites can cause a variety of diseases or symptoms, including, but not limited to: Scabies, Trombiculiasis, eye infections, intestinal disease (e.g., dysentery, giardiasis), liver disease, lung disease, opportunistic infections (e.g., AIDS related), Malaria, pregnancy complications, and toxoplasmosis. VEGF-3 polypeptides or polynucleotides can be used to treat or detect any of these symptoms or diseases.

Preferably, treatment using VEGF-3 polypeptides or polynucleotides could either be by administering an effective amount of VEGF-3 polypeptide to the patient, or by removing cells from the patient, supplying the cells with VEGF-3 polynucleotide, and returning the engineered cells to the patient (exvivo therapy). Moreover, the VEGF-3 polypeptide or polynucleotide can be used as an antigen in a vaccine to raise an immune response against infectious disease.

Regeneration

VEGF-3 polynucleotides or polypeptides can be used to differentiate, proliferate, and attract cells, leading to the regeneration of tissues. (See, Science 276:59-87 (1997).) The regeneration of tissues could be used to repair, replace, or protect tissue damaged by congenital defects, trauma (wounds, bums, incisions, or ulcers), age, disease (e.g. osteoporosis, osteocarthritis, periodontal disease, liver failure), surgery, including cosmetic plastic surgery, fibrosis, reperfusion injury, or systemic cytokine damage.

Tissues that could be regenerated using the present invention include organs (e.g., pancreas, liver, intestine, kidney, skin, endothelium), muscle (smooth, skeletal or cardiac), vascular (including vascular endothelium), nervous, hematopoietic, and skeletal (bone, cartilage, tendon, and ligament) tissue. Preferably, regeneration occurs without or decreased scarring. Regeneration also may include angiogenesis.

Moreover, VEGF-3 polynucleotides or polypeptides may increase regeneration of tissues difficult to heal. For example, increased tendon/ligament regeneration would quickenrecoverytime after damage. VEGF-3 polynucleotides or polypeptides of the present invention could also be used prophylactically in an effort to avoid damage. Specific diseases that could be treated include of tendinitis, carpal tunnel syndrome, and other tendon or ligament defects. A further example of tissue regeneration of non-healing wounds includes pressure ulcers, ulcers associated with vascular insufficiency, surgical, and traumatic wounds.

Similarly, nerve and brain tissue could also be regenerated by using VEGF-3 polynucleotides or polypeptides to proliferate and differentiate nerve cells. Diseases that could be treated using this method include central and peripheral nervous system diseases, neuropathies, or mechanical and traumatic disorders (e.g., spinal cord disorders, head trauma, cerebrovascular disease, and stoke). Specifically, diseases associated with peripheral nerve injuries, peripheral neuropathy (e.g., resulting from chemotherapy or other medical therapies), localized neuropathies, and central nervous system diseases (e.g., Alzheimer's disease, Parkinson's disease, Huntington's disease, amyotrophic lateral sclerosis, and Shy-Drager syndrome), could all be treated using the VEGF-3 polynucleotides or polypeptides.

Chemotaxis

VEGF-3 polynucleotides or polypeptides may have chemotaxis activity. A chemotaxic molecule attracts or mobilizes cells (e.g., monocytes, fibroblasts, neutrophils, T-cells, mast cells, eosinophils, epithelial and/or endothelial cells) to a particular site in the body, such as inflammation, infection, or site of hyperproliferation. The mobilized cells can then fight offand/or heal the particular trauma or abnormality.

VEGF-3 polynucleotides or polypeptides may increase chemotaxic activity of particular cells. These chemotactic molecules can then be used to treat inflammation, infection, hyperproliferative disorders, or any immune system disorder by increasing the number of cells targeted to a particular location in the body. For example, chemotaxic molecules can be used to treat wounds and other trauma to tissues by attracting immune cells to the injured location. As a chemotactic molecule, VEGF-3 could also attract fibroblasts, which can be used to treat wounds.

It is also contemplated that VEGF-3 polynucleotides or polypeptides may inhibit chemotactic activity. These molecules could also be used to treat disorders. Thus, VEGF-3 polynucleotides or polypeptides could be used as an inhibitor of chemotaxis.

Binding Activity

VEGF-3 polypeptides may be used to screen for molecules that bind to VEGF-3 or for molecules to which VEGF-3 binds. The binding of VEGF-3 and the molecule may activate (agonist), increase, inhibit (antagonist), or decrease activity of the VEGF-3 or the molecule bound. Examples of such molecules include antibodies, oligonucleotides, proteins (e.g., receptors), or small molecules.

Preferably, the molecule is closely related to the natural ligand ofVEGF-3, e.g., a fragment of the ligand, or a natural substrate, a ligand, a structural or functional mimetic. (See, Coligan et al., Current Protocols in Immunology 1(2):Chapter 5 (1991).) Similarly, the molecule can be closely related to the natural receptor to which VEGF-3 binds, or at least, a fragment of the receptor capable of being bound by VEGF-3 (e.g., active site). In either case, the molecule can be rationally designed using known techniques.

Preferably, the screening for these molecules involves producing appropriate cells which express VEGF-3, either as a secreted protein or on the cell membrane. Preferred cells include cells from mammals, yeast, Drosophila, or E. coli. Cells expressing VEGF-3(or cell membrane containing the expressed polypeptide) are then preferably contacted with a test compound potentially containing the molecule to observe binding, stimulation, or inhibition of activity of either VEGF-3 or the molecule.

The assay may simply test binding of a candidate compound to VEGF-3, wherein binding is detected by a label, or in an assay involving competition with a labeled competitor. Further, the assay may test whether the candidate compound results in a signal generated by binding to VEGF-3.

Alternatively, the assay can be carried out using cell-free preparations, polypeptide/molecule affixed to a solid support, chemical libraries, or natural product mixtures. The assay may also simply comprise the steps of mixing a candidate compound with a solution containing VEGF-3, measuring VEGF-3/molecule activity or binding, and comparing the VEGF-3/molecule activity or binding to a standard.

Preferably, an ELISA assay can measure VEGF-3 level or activity in a sample (e.g., biological sample) using a monoclonal or polyclonal antibody. The antibody can measure VEGF-3 level or activity by either binding, directly or indirectly, to VEGF-3 or by competing with VEGF-3 for a substrate.

All of these above assays can be used as diagnostic or prognostic markers. The molecules discovered using these assays can be used to treat disease or to bring about a particular result in a patient (e.g., blood vessel growth) by activating or inhibiting the VEGF-3/molecule. Moreover, the assays can discover agents which may inhibit or enhance the production of VEGF-3 from suitably manipulated cells or tissues.

Therefore, the invention includes a method of identifying compounds whichbind to VEGF-3 comprising the steps of: (a) incubating a candidate binding compound with VEGF-3; and (b) determining if binding has occurred. Moreover, the invention includes a method of identifying agonists/antagonists comprising the steps of: (a) incubating a candidate compound with VEGF-3, (b) assaying a biological activity, and (b) determining if a biological activity ofVEGF-3 has been altered.

Other Activities

VEGF-3 polypeptides or polynucleotides may also increase or decrease the differentiation or proliferation of embryonic stem cells, besides, as discussed above, hematopoietic lineage.

VEGF-3 polypeptides or polynucleotides may also be used to modulate mammalian characteristics, such as body height, weight, hair color, eye color, skin, percentage of adipose tissue, pigmentation, size, and shape (e.g., cosmetic surgery). Similarly, VEGF-3 polypeptides or polynucleotides may be used to modulate mammalian metabolism affecting catabolism, anabolism, processing, utilization, and storage of energy. VEGF-3 polypeptides or polynucleotides may be used to change a mammal's mental state or physical state by influencing biorhythms, caricadic rhythms, depression (including depressive disorders), tendency for violence, tolerance for pain, reproductive capabilities (preferably by Activin or Inhibin-like activity), hormonal or endocrine levels, appetite, libido, memory, stress, or other cognitive qualities.

VEGF-3 polypeptides or polynucleotides may also be used as a food additive or preservative, such as to increase or decrease storage capabilities, fat content, lipid, protein, carbohydrate, vitamins, minerals, cofactors or other nutritional components.

Having generally described the invention, the same will be more readily understood by reference to the following examples, which are provided by way of illustration and are not intended as limiting.

### EXAMPLES

### Example 1

Isolation of the VEGF-3 cDNA Clone from the Deposited Sample

The cDNA for VEGF-3 is inserted into the multiple cloning site ofpQE-70 (Qiagen, Inc., Chatsworth, Calif.). pQE-70 contains an antibiotic resistance gene (Ampr) and may be transformed into E. coli strain DH10B, available from Life Technologies. (See, for instance, Gruber, C. E., et al., Focus 15:59(1993).) Two approaches can be used to isolate VEGF-3 from the deposited sample. First, a specific polynucleotide of SEQ ID NO:1 with 30-40 nucleotides is synthesized using an Applied Biosystems DNA synthesizer according to the sequence reported. The oligonucleotide is labeled, for instance, with ³²P-g-ATP using T4 polynucleotide kinase and purified according to routine methods. (E.g., Maniatis et al., Molecular Cloning: A Laboratory Manual, Cold Spring Harbor Press, Cold Spring, N.Y. (1982).) The plasmid mixture is transformed into a suitable host (such as XL-1 Blue (Stratagene)) using techniques known to those of skill in the art, such as those provided by the vector supplier or in related publications or patents. The transformants are plated on 1.5% agar plates (containing the appropriate selection agent, e.g., ampicillin) to a density of about 150 transformants (colonies) per plate. These plates are screened using Nylon membranes according to routine methods for bacterial colony screening (e.g., Sambrook et al., Molecular Cloning: A Laboratory Manual, 2nd Ed., (1989), Cold Spring Harbor Laboratory Press, pages 1.93 to 1.104), or other techniques known to those of skill in the art.

Alternatively, two primers of 17-20 nucleotides derived from both ends of the SEQ ID NO:1 (i.e., within the region of SEQ ID NO:1 bounded by the 5′ NT and the 3′ NT of the clone) are synthesized and used to amplify the VEGF-3 cDNA using the deposited cDNA plasmid as a template. The polymerase chain reaction is carried out under routine conditions, for instance, in 25 μl of reaction mixture with 0.5 μg of the above cDNA template. A convenient reaction mixture is 1.5-5 mM MgCl₂, 0.01% (w/v) gelatin, 20 μM each of dATP, dCTP, dGTP, dTTP, 25 pmol of each primer and 0.25 Unit of Taq polymerase. Thirty five cycles of PCR (denaturation at 94° C. for 1 min; annealing at 55° C. for 1 min; elongation at 72° C. for 1 min) are performed with a Perkin-Elmer Cetus automated thermal cycler. The amplified product is analyzed by agarose gel electrophoresis and the DNA band with expected molecular weight is excised and purified. The PCR product is verified to be the selected sequence by subcloning and sequencing the DNA product.

Several methods are available for the identification of the 5′ or 3′ non-coding portions of the VEGF-3 gene which may not be present in the deposited clone. These methods include but are not limited to, filter probing, clone enrichment using specific probes, and protocols similar or identical to 5′ and 3 “RACE” protocols which are well known in the art. For instance, a method similar to 5′ RACE is available for generating the missing 5′ end of a desired full-length transcript. (Fromont-Racine et al., Nucleic Acids Res. 21(7):1683-1684 (1993).)

Briefly, a specific RNA oligonucleotide is ligated to the 5′ ends of a population of RNA presumably containing full-length gene RNA transcripts. A primer set containing a primer specific to the ligated RNA oligonucleotide and a primer specific to a known sequence of the VEGF-3 gene of interest is used to PCR amplify the 5′ portion of the VEGF-3 full-length gene. This amplified product may then be sequenced and used to generate the full length gene.

This above method starts with total RNA isolated from the desired source, although poly-A+RNA can be used. The RNA preparation can then be treated with phosphatase if necessary to eliminate 5′ phosphate groups on degraded or damaged RNA which may interfere with the later RNA ligase step. The phosphatase should then be inactivated and the RNA treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase.

This modified RNA preparation is used as a template for first strand cDNA synthesis using a gene specific oligonucleotide. The first strand synthesis reaction is used as a template for PCR amplification of the desired 5′ end using a primer specific to the ligated RNA oligonucleotide and a primer specific to the known sequence of the gene of interest. The resultant product is then sequenced and analyzed to confirm that the 5′ end sequence belongs to the VEGF-3 gene.

### Example 2

Isolation of VEGF-3 Genomic Clones

A human genomic P1 library (Genomic Systems, Inc.) is screened by PCR using primers selected for the cDNA sequence corresponding to SEQ ID NO:1., according to the method described in Example 1. (See also, Sambrook.)

### Example 3

Tissue Distribution of VEGF-3 Polypeptides

Tissue distribution of mRNA expression of VEGF-3 is determined using protocols for Northern blot analysis, described by, among others, Sambrook et al. For example, a VEGF-3 probe produced by the method described in Example 1 is labeled with p³² using the rediprimea DNA labeling system (Amersham Life Science), according to manufacturer's instructions. After labeling, the probe is purified using CHROMA SPIN-100^{a} column (Clontech Laboratories, Inc.), according to manufacturer's protocol number PT1200-1. The purified labeled probe is then used to examine various human tissues for mRNA expression.

Multiple Tissue Northern (MTN) blots containing various human tissues (H) or human immune system tissues (IM) (Clontech) are examined with the labeled probe using ExpressHyba hybridization solution (Clontech) according to manufacturer's protocol number PT 1190-1. Following hybridization and washing, the blots are mounted and exposed to film at −70° C. overnight, and the films developed according to standard procedures.

### Example 4

Chromosomal Mapping of VEGF-3

An oligonucleotide primer set is designed according to the sequence at the 5′ end of SEQ ID NO:1 or SEQ ID NO:19. This primer preferably spans about 100 nucleotides. This primer set is then used in a polymerase chain reaction under the following set of conditions: 30 seconds, 95° C.; 1 minute, 56° C.; 1 minute, 70° C. This cycle is repeated 32 times followed by one 5 minute cycle at 70° C. Human, mouse, and hamster DNA is used as template in addition to a somatic cell hybrid panel containing individual chromosomes or chromosome fragments (Bios, Inc). The reactions is analyzed on either 8% polyacrylamide gels or 3.5% agarose gels. Chromosome mapping is determined by the presence of an approximately 100 bp PCR fragment in the particular somatic cell hybrid.

### Example 5

Bacterial Expression of VEGF-3

VEGF-3 polynucleotide encoding a VEGF-3 polypeptide invention is amplified using PCR oligonucleotide primers corresponding to the 5′ and 3′ ends of the DNA sequence, as outlined in Example 1, to synthesize insertion fragments. The primers used to amplify the cDNA insert should preferably contain restriction sites, such as BamHI and XbaI, at the 5′ end of the primers in order to clone the amplified product into the expression vector. For example, BamHI and XbaI correspond to the restriction enzyme sites on the bacterial expression vector pQE-9. (Qiagen, Inc., Chatsworth, Calif,). This plasmid vector encodes antibiotic resistance (Amp), a bacterial origin of replication (ori), an IPTG-regulatable promoter/operator (P/O), a ribosome binding site (RB S), a 6-histidine tag (6-His), and restriction enzyme cloning sites.

Specifically, to clone the VEGF-3 protein in a bacterial vector, the 5′ primer has the sequence 5′ GACTGCATGCACCAGAGGAAAGTGGTGTC 3′ (SEQ ID NO:5), which contains a SphI restriction enzyme site followed by VEGF3 coding sequence starting from the presumed terminal amino acid of the processed protein codon. One of ordinary skill in the art would appreciate, of course, that the point in the protein coding sequence where the 5′ primer begins may be varied to amplify a DNA segment encoding any desired portion of the complete VEGF-3 protein shorter or longer than the complete protein. The 3′ primer has the sequence 5′ GACTAGATCTCCTTCGCAGCTTCCGGCAC 3′ (SEQ ID NO:6) contains complementary sequences to BhlII site located 3'to the VEGF3 DNA insert.

The pQE-9 vector is digested with BamHI and XbaI and the amplified fragment is ligated into the pQE-9 vector maintaining the reading frame initiated at the bacterial RB S. The ligation mixture is then used to transform the E. Coli strain M15/rep4 (Qiagen, Inc.) which contains multiple copies of the plasmid pREP4, which expresses the lacI repressor and also confers kanamycin resistance (Kanr). Transformants are identified by their ability to grow on LB plates and ampicillin/kanamycin resistant colonies are selected. Plasmid DNA is isolated and confirmed by restriction analysis.

Clones containing the desired constructs are grown overnight (O/N) in liquid culture in LB media supplemented with both Amp (100 μg/ml) and Kan (25 μg/ml). The O/N culture is used to inoculate a large culture at a ratio of 1: 100 to 1:250. The cells are grown to an optical density 600 (O.D.⁶⁰⁰) ofbetween 0.4 and 0.6. IPTG (Isopropyl-B-D-thiogalacto pyranoside) is then added to a final concentration of 1 mM. IPTG induces by inactivating the lacI repressor, clearing the P/O leading to increased gene expression.

Cells are grown for an extra 3 to 4 hours. Cells are then harvested by centrifugation (20 mins at 6000× g). The cell pellet is solubilized in the chaotropic agent 6 Molar Guanidine HCl by stirring for 3-4 hours at 4° C. The cell debris is removed by centrifugation, and the supernatant containing the polypeptide is loaded onto a nickel-nitrilo-tri-acetic acid (“Ni-NTA”) affinity resin column (available from QIAGEN, Inc., supra). Proteins with a 6 × His tag bind to the Ni-NTA resin with high affinity and can be purified in a simple one-step procedure (for details see: The QlAexpressionist (1995) QIAGEN, Inc., supra).

Briefly, the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the polypeptide is eluted with 6 M guanidine-HCl, pH 5.

The purified VEGF-3 protein is then renatured by dialyzing it against phosphate-buffered saline (PBS) or 50 mM Na-acetate, pH 6 buffer plus 200 mM NaCl. Alternatively, the VEGF-3 protein can be successfully refolded while immobilized on the Ni-NTA column. The recommended conditions are as follows: renature using a linear 6M-1M urea gradient in 500 mM NaCl, 20% glycerol, 20 mM Tris/HCl pH 7.4, containing protease inhibitors. The renaturation should be performed over a period of 1.5 hours or more. After renaturation the proteins are eluted by the addition of 250 mnM immidazole. Immidazole is removed by a final dialyzing step against PBS or 50 mM sodium acetate pH 6 buffer plus 200 mM NaCl. The purified VEGF-3 protein is stored at 4° C. or frozen at −80 C.

In addition to the above expression vector, the present invention further includes an expression vector comprising phage operator and promoter elements operatively linked to a VEGF-3 polynucleotide, called pHE4a. (ATCC Accession Number 209645, deposited Feb. 25, 1998.) This vector contains: 1) a neomycinphosphotransferase gene as a selection marker, 2) an E. coli origin of replication, 3) a T5 phage promoter sequence, 4) two lac operator sequences, 5) a Shine-Delgarno sequence, and 6) the lactose operon repressor gene (lacIq). The origin ofreplication (oriC) is derived from pUC 19 (LTI, Gaithersburg, Md.). The promoter sequence and operator sequences are made synthetically.

DNA can be inserted into the pHEa by restricting the vector with NdeI and XbaI, BamHI, XhoI, or Asp718, running the restricted product on a gel, and isolating the larger fragment (the stuffer fragment should be about 310 base pairs). The DNA insert is generated according to the PCR protocol described in Example 1, using PCR primers having restriction sites for NdeI (5′ primer) and XbaI, BamHI, XhoI, or Asp718 (3 ′ primer). The PCR insert is gel purified and restricted with compatible enzymes. The insert and vector are ligated according to standard protocols.

The engineered vector could easily be substituted in the above protocol to express protein in a bacterial system.

### Example 6

Purification of VEGF-3 Polypeptide from an Inclusion Body

The following alternative method can be used to purify VEGF-3 polypeptide expressed in E coli when it is present in the form of inclusion bodies. Unless otherwise specified, all of the following steps are conducted at 4-10° C.

Upon completion of the production phase of the E. coli fermentation, the cell culture is cooled to 4-10° C. and the cells harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (Microfuidics, Corp. or APV Gaulin, Inc.) twice at 4000-6000 psi. The homogenate is then mixed with NaCl solution to a final concentration of 0.5 M NaCl, followed by centrifugation at 7000× g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000× g centrifugation for 15 min., the pellet is discarded and the polypeptide containing supernatant is incubated at 4° C. overnight to allow further GuHCl extraction.

Following high speed centrifugation (30,000× g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4° C. without mixing for 12 hours prior to further purification steps.

To clarify the refolded polypeptide solution, a previously prepared tangential filtration unit equipped with 0.16 mm membrane filter with appropriate surface area (e.g., Filtron), equilibrated with 40 mM sodium acetate, pH 6.0 is employed. The filtered sample is loaded onto a cation exchange resin (e.g., Poros HS-50, Perseptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 nm of the effluent is continuously monitored. Fractions are collected and further analyzed by SDS-PAGE.

Fractions containing the VEGF-3 polypeptide are then pooled and mixed with 4 volumes of water. The diluted sample is then loaded onto a previously prepared set of tandem columns of strong anion (Poros HQ-50, Perseptive Biosystems) and weak anion (Poros CM-20, Perseptive Biosystems) exchange resins. The columns are equilibrated with 40 mM sodium acetate, pH 6.0. Both columns are washed with 40 mM sodium acetate, pH 6.0, 200 MM NaCl. The CM-20 column is then eluted using a 10 column volume linear gradient ranging from 0.2 M NaCl, 50 mM sodium acetate, pH 6.0 to 1.0 M NaCl, 50 MM sodium acetate, pH 6.5. Fractions are collected under constant A₂₈₀ monitoring of the effluent. Fractions containing the polypeptide (determined, for instance, by 16% SDS-PAGE) are then pooled.

The resultant VEGF-3 polypeptide should exhibit greater than 95% purity after the above refolding and purification steps. No major contaminant bands should be observed from Coomassie blue stained 16% SDS-PAGE gel when 5 mg of purified protein is loaded. The purified VEGF-3 protein can also be tested for endotoxin/LPS contamination, and typically the LPS content is less than 0.1 ng/ml according to LAL assays.

### Example 7

Cloning and Expression of VEGF-3 in a Baculovirus Expression System

In this example, the plasmid shuttle vector pA2 is used to insert VEGF-3 polynucleotide into a baculovirus to express VEGF-3. This expression vector contains the strong polyhedrin promoter of the Autographa californica nuclear polyhedrosis virus (AcMNPV) followed by convenient restriction sites such as BamHI, XbaI and Asp718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned VEGF-3 polynucleotide.

Many other baculovirus vectors can be used in place of the vector above, such as pAc373, pVL941, and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

Specifically, the VEGF-3 cDNA sequence contained in the deposited clone, including the AUG initiation codon and any naturally associated leader sequence, is amplified using the PCR protocol described in Example 1. If the naturally occurring signal sequence is used to produce the secreted protein, the pA2 vector does not need a second signal peptide. Alternatively, the vector can be modified (pA2 GP) to include a baculovirus leader sequence, using the standard methods described in Summers et al., “A Manual of Methods for Baculovirus Vectors and Insect Cell Culture Procedures,” Texas Agricultural Experimental Station Bulletin No. 1555 (1987).

More specifically, the cDNA sequence encoding the full length VEGF-3 protein in the deposited clone, including the AUG initiation codon and the naturally associated leader sequence shown in SEQ ID NO:1, is amplified using PCR oligonucleotide primers corresponding to the 5′ and 3′ sequences of the gene. The 5′ primer has the sequence 5′ GCA TGG ATC CCA GCC TGA TGC CCC TGG CC (SEQ ID NO:7) and contains a BamH1 restriction enzyme site and nucleotide sequence complementary to the 5′ sequence of VEGF3.

The 3′ primer has the sequence 5′ GCA TTC TAG ACC CTG CTG AGT CTG AAA AGC 3′ (SEQ ID NO:8) and contains the cleavage site for the restriction enzyme XbaI and nucleotides complementary to the 3′ sequence of VEGF3.

The amplified fragment is isolated from a 1% agarose gel using a commercially available kit (“Geneclean,” BIO 101 Inc., La Jolla, Calif.). The fragment then is digested with appropriate restriction enzymes and again purified on a 1% agarose gel.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.).

The fragment and the dephosphorylated plasmid are ligated together with T4 DNA ligase. E. coli B 101 or other suitable E. coli hosts such as XL-1 Blue (Stratagene Cloning Systems, La Jolla, Calif.) cells are transformed with the ligation mixture and spread on culture plates. Bacteria containing the plasmid are identified by digesting DNA from individual colonies and analyzing the digestion product by gel electrophoresis. The sequence of the cloned fragment is confirmed by DNA sequencing.

Five μg of a plasmid containing the polynucleotide is co-transfected with 1.0 μg of a commercially available linearized baculovirus DNA (“BaculoGold™ baculovirus DNA”, Pharmingen, San Diego, Calif.), using the lipofection method described by Felgner et al., Proc. Natl. Acad. Sci. USA 84:7413-7417 (1987). One μg of BaculoGold^{a} virus DNA and 5 μg of the plasmid are mixed in a sterile well of a microtiter plate containing 50 μl of serum-free Grace's medium (Life Technologies Inc., Gaithersburg, Md.). Afterwards, 10 μl Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27° C. The transfection solution is then removed from the plate and 1 ml of Grace's insect medium supplemented with 10% fetal calf serum is added. Cultivation is then continued at 27° C. for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10.) After appropriate incubation, blue stained plaques are picked with the tip of a micropipettor (e.g., Eppendorf). The agar containing the recombinant viruses is then resuspended in a microcentrifuge tube containing 200 μl of Grace's medium and the suspension containing the recombinant baculovirus is used to infect Sf9 cells seeded in 35 mm dishes. Four days later the supernatants of these culture dishes are harvested and then they are stored at 4° C.

To verify the expression of the polypeptide, Sf9 cells are grown in Grace's medium supplemented with 10% heat-inactivated FBS. The cells are infected with the recombinant baculovirus containing the polynucleotide at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 μCi of ³⁵S-methionine and 5 μCi ³⁵S-cysteine (available from Amersham) are added. The cells are further incubated for 16 hours and then are harvested by centrifigation. The proteins in the supernatant as well as the intracellular proteins are analyzed by SDS-PAGE followed by autoradiography (if radiolabeled). Microsequencing of the amino acid sequence of the amino terminus of purified protein may be used to determine the amino terminal sequence of the produced VEGF-3 protein.

### Example 8

Expression of VEGF-3 in Mammalian Cells

VEGF-3 polypeptide can be expressed in a mammalian cell. A typical mammalian expression vector contains a promoter element, which mediates the initiation of transcription of MRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter).

Suitable expression vectors for use in practicing the present invention include, for example, vectors such as pSVL and pMSG (Pharmacia, Uppsala, Sweden), pRSVcat (ATCC 37152), pSV2dhfr (ATCC 37146), pBC 12MI (ATCC 67109), pCMVSport 2.0, and pCMVSport 3.0. Mammalian host cells that could be used include, human Hela, 293, H9 and Jurkat cells, mouse NIH3 T3 and C 127 cells, Cos 1, Cos 7 and CV1, quail QC1-3 cells, mouse L cells and Chinese hamster ovary (CHO) cells.

Alternatively, VEGF-3 polypeptide can be expressed in stable cell lines containing the VEGF-3 polynucleotide integrated into a chromosome. The co-transfection with a selectable marker such as dhfr, gpt, neomycin, hygromycin allows the identification and isolation of the transfected cells.

The transfected VEGF-3 gene can also be amplified to express large amounts of the encoded protein. The DHFR (dihydrofolate reductase) marker is useful in developing cell lines that carry several hundred or even several thousand copies of the gene of interest. (See, e.g., Alt, F. W. et al., J. Biol. Chem. 253:1357-1370 (1978); Hamlin, J. L. and Ma, C., Biochem. et Biophys. Acta 1097:107-143 (1990); Page, M. J. and Sydenham, M. A., Biotechnology 9:64-68 (1991).) Another useful selection marker is the enzyme glutamine synthase (GS) (Murphy et al., Biochem J. 227:277-279 (1991); Bebbington et al., Bio/Technology 10:169-175 (1992). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

Derivatives of the plasmid pSV2-dhfr (ATCC Accession No.37146), the expression vectors pC4 (ATCC Accession No. 209646) and pC6 (ATCC Accession No.209647) contain the strong promoter (LTR) of the Rous Sarcoma Virus (Cullen et al., Molecular and Cellular Biology, 438-447 (March, 1985)) plus a fragment of the CMV-enhancer (Boshart et al., Cell 41:521-530 (1985).) Multiple cloning sites, e.g., with the restriction enzyme cleavage sites BamHI, XbaI and Asp718, facilitate the cloning of VEGF-3. The vectors also contain the 3′ intron, the polyadenylation and termination signal of the rat preproinsulin gene, and the mouse DHFR gene under control of the SV40 early promoter. Specifically, the plasmid pC6, for example, is digested with appropriate restriction enzymes and then dephosphorylated using calfintestinal phosphates by procedures known in the art. The vector is then isolated from a 1% agarose gel.

VEGF-3 polynucleotide is amplified according to the protocol outlined in Example 1. If the naturally occurring signal sequence is used to produce the secreted protein, the vector does not need a second signal peptide. Alternatively, if the naturally occurring signal sequence is not used, the vector can be modified to include a heterologous signal sequence. (See, e.g., WO 96/34891.) The amplified fragment is isolated from a 1% agarose gel using a commercially available kit (“Geneclean,” BIO 101 Inc., La Jolla, Calif.). The fragment then is digested with appropriate restriction enzymes and again purified on a 1% agarose gel.

The amplified fragment is then digested with the same restriction enzyme and purified on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC6 using, for instance, restriction enzyme analysis.

Chinese hamster ovary cells lacking an active DHFR gene is used for transfection. Five μg of the expression plasmid pC6 is cotransfected with 0.5 μg of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus 1 mg/mil G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 μM, 2 μM, 5 μM, 10 mM, 20 mM). The same procedure is repeated until clones are obtained which grow at a concentration of 100-200 μM. Expression of VEGF-3 is analyzed, for instance, by SD S-PAGE and Western blot or by reversed phase BPLC analysis.

### Example 9

Construction of N-terminal and/or C-terminal Deletion Mutants

The following general approach may be used to clone a N-terminal or C-terminal deletion VEGF-3 deletion mutant. Generally, two oligonucleotide primers of about 15-25 nucleotides are derived from the desired 5′ and 3′ positions of a polynucleotide of SEQ ID NO:1. The 5′ and 3′ positions of the primers are determined based on the desired VEGF-3 polynucleotide fragment. An initiation and stop codon are added to the 5′ and 3′ primers respectively, if necessary, to express the VEGF-3 polypeptide fragment encoded by the polynucleotide fragment. Preferred VEGF-3 polynucleotide fragments are those encoding the N-terminal and C-terminal deletion mutants disclosed above inthe “Polynucleotide and Polypeptide Fragments” section of the Specification.

Additional nucleotides containing restriction sites to facilitate cloning of the VEGF-3 polynucleotide fragment in a desired vector may also be added to the 5′ and 3′ primer sequences. The VEGF-3 polynucleotide fragment is amplified from genomic DNA or from the deposited cDNA clone using the appropriate PCR oligonucleotide primers and conditions discussed herein or known in the art. The VEGF-3 polypeptide fragments encoded by the VEGF-3 polynucleotide fragments of the present invention may be expressed and purified in the same general manner as the full length polypeptides, although routine modifications may be necessary due to the differences in chemical and physical properties between a particular fragment and full length polypeptide.

As a means of exemplifying but not limiting the present invention, the polynucleotide encoding the VEGF-3 polypeptide fragment V-35 to A-131 is amplified and cloned as follows: A 5′ primer is generated comprising a restriction enzyme site followed by an initiation codon in frame with the polynucleotide sequence encoding the N-terminal portion of the polypeptide fragment beginning with V-35. A complementary 3′ primer is generated comprising a restriction enzyme site followed by a stop codon in frame with the polynucleotide sequence encoding C-terminal portion of the VEGF-3 polypeptide fragment ending with A-131.

The amplified polynucleotide fragment and the expression vector are digested with restriction enzymes which recognize the sites in the primers. The digested polynucleotides are then ligated together. The VEGF-3 polynucleotide fragment is inserted into the restricted expression vector, preferably in a manner which places the VEGF-3 polypeptide fragment coding region downstream from the promoter. The ligation mixture is transformed into competent E. coli cells using standard procedures and as described in the Examples herein. Plasmid DNA is isolated from resistant colonies and the identity of the cloned DNA confirmed by restriction analysis, PCR and DNA sequencing.

### Example 10

Protein Fusions of VEGF-3

VEGF-3 polypeptides are preferably fused to other proteins. These fusion proteins can be used for a variety of applications. For example, fusion of VEGF-3 polypeptides to His-tag, HA-tag, protein A, IgG domains, and maltose binding protein facilitates purification. (See Example 5; see also EP A 394,827; Traunecker et al., Nature 331:84-86 (1988).) Similarly, fusion to IgG-1, IgG-3, and albumin increases the halflife time in vivo. Nuclear localization signals fused to VEGF-3 polypeptides can target the protein to a specific subcellular localization, while covalent heterodimer or homodimers can increase or decrease the activity of a fusion protein. Fusion proteins can also create chimeric molecules having more than one function. Finally, fusion proteins can increase solubility and/or stability of the fused protein compared to the non-fused protein. All of the types of fusion proteins described above can be made by modifying the following protocol, which outlines the fusion of a polypeptide to an IgG molecule, or the protocol described in Example 5 .

Briefly, the human Fc portion of the IgG molecule can be PCR amplified, using primers that span the 5′ and 3′ ends of the sequence described below. These primers also should have convenient restriction enzyme sites that will facilitate cloning into an expression vector, preferably a mammalian expression vector.

For example, if pC4 (Accession No. 209646) is used, the human Fc portion can be ligated into the BamHI cloning site. Note that the 3′ BamHI site should be destroyed. Next, the vector containing the human Fc portion is re-restricted with BamHI, linearizing the vector, and VEGF-3 polynucleotide, isolated by the PCR protocol described in Example 1, is ligated into this BamHI site. Note that the polynucleotide is cloned without a stop codon, otherwise a fusion protein will not be produced.

If the naturally occurring signal sequence is used to produce the secreted protein, pC4 does not need a second signal peptide. Alternatively, if the naturally occurring signal sequence is not used, the vector can be modified to include a heterologous signal sequence. (See, e.g., WO 96/34891.)

| | |
|---|---|
| Human IgG Fc region: | |
| GGGATCCGGAGCCCAAATCTTCTGACAAAACTCACACATGCCCACCGTGCCCAG | (SEQ ID NO: 9) |
| CACCTGAATTCGAGGGTGCACCGTCAGTCTTCCTCTTCCCCCCAAAACCCAAGGA | |
| CACCCTCATGATCTCCCGGACTCCTGAGGTCACATGCGTGGTGGTGGACGTAAGC | |
| CACGAAGACCCTGAGGTCAAGTTCAACTGGTACGTGGACGGCGTGGAGGTGCAT | |
| AATGCCAAGACAAAGCCGCGGGAGGAGCAGTACAACAGCACGTACCGTGTGGTC | |
| AGCGTCCTCACCGTCCTGCACCAGGACTGGCTGAATGGCAAGGAGTACAAGTGC | |
| AAGGTCTCCAACAAAGCCCTCCCAACCCCCATCGAGAAAACCATCTCCAAAGCC | |
| AAAGGGCAGCCCCGAGAACCACAGGTGTACACCCTGCCCCCATCCCGGGATGAG | |
| CTGACCAAGAACCAGGTCAGCCTGACCTGCCTGGTCAAAGGCTTCTATCCAAGC | |
| GACATCGCCGTGGAGTGGGAGAGCAATGGGCAGCCGGAGAACAACTACAAGAC | |
| CACGCCTCCCGTGCTGGACTCCGACGGCTCCTTCTTCCTCTACAGCAAGCTCACC | |
| GTGGACAAGAGCAGGTGGCAGCAGGGGAACGTCTTCTCATGCTCCGTGATGCAT | |
| GAGGCTCTGCACAACCACTACACGCAGAAGAGCCTCTCCCTGTCTCCGGGTAAAT | |
| GAGTGCGACGGCCGCGACTCTAGAGGAT | |

### Example 11

Production of an Antibody

The antibodies of the present invention can be prepared by a variety of methods. (See, Current Protocols, Chapter 2.) For example, cells expressing VEGF-3 is administered to an animal to induce the production of sera containing polyclonal antibodies. In a preferred method, a preparation of VEGF-3 protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or protein binding fragments thereof). Such monoclonal antibodies can be prepared using hybridoma technology. (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., pp. 563-681 (1981).) In general, such procedures involve immunizing an animal (preferably a mouse) with VEGF-3 polypeptide or, more preferably, with a secreted VEGF-3 polypeptide-expressing cell. Such cells may be cultured in any suitable tissue culture medium; however, it is preferable to culture cells in Earle's modified Eagle's medium supplemented with 10% fetal bovine serum (inactivated at about 56° C.), and supplemented with about 10 g/l of nonessential amino acids, about 1,000 U/ml of penicillin, and about 100 μg/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP20), available from the ATCC. After fusion, the resulting hybridoma cells are selectively maintained in HAT medium, and then cloned by limiting dilution as described by Wands et al. (Gastroenterology 80:225-232(1981).) The hybridoma cells obtained through such a selection are then assayed to identify clones which secrete antibodies capable of binding the VEGF-3 polypeptide.

Alternatively, additional antibodies capable of binding to VEGF-3 polypeptide can be produced in a two-step procedure using anti-idiotypic antibodies. Such a method makes use of the fact that antibodies are themselves antigens, and therefore, it is possible to obtain an antibody which binds to a second antibody. In accordance with this method, protein specific antibodies are used to immunize an animal, preferably a mouse. The splenocytes of such an animal are then used to produce hybridoma cells, and the hybridoma cells are screened to identify clones which produce an antibody whose ability to bind to the VEGF-3 protein-specific antibody can be blocked byVEGF-3. Such antibodies comprise anti-idiotypic antibodies to the VEGF-3 protein-specific antibody and can be used to immunize an animal to induce formation of further VEGF-3 protein-specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)2 fragments). Alternatively, secreted VEGF-3 protein-binding fragments can be produced through the application of recombinant DNA technology or through synthetic chemistry.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are knownintheart. (See, for review, Morrison, Science 229:1202(1985); Oi et al., BioTechniques4:214 (1986); Cabilly et al., U.S. Pat. No. 4,816,567; Taniguchi et al., EP 171496; Morrison et al., EP 173494; Neuberger et al., WO 8601533; Robinson et al, WO 8702671; Boulianne et al., Nature 312:643 (1984); Neuberger et al., Nature 314:268 (1985).).

### Example 12

Production Of VEGF-3 Protein For High-throughput Screening Assays

The following protocol produces a supernatant containing VEGF-3 polypeptide to be tested. This supernatant can then be used in the Screening Assays described in Examples 14-21.

First, dilute Poly-D-Lysine (644 587 Boehringer-Mannheim) stock solution (1 mg/ml in PBS) 1:20 in PBS (w/o calcium or magnesium 17-516F Biowhittaker) for a working solution of 50 ug/ml. Add 200 ul of this solution to each well (24 well plates) and incubate at RT for 20 minutes. Be sure to distribute the solution over each well (note: a 12-channel pipetter may be used with tips on every other channel). Aspirate off the Poly-D-Lysine solution and rinse with 1 ml PBS (Phosphate Buffered Saline). The PBS should remain in the well until just prior to plating the cells and plates may be poly-lysine coated in advance for up to two weeks.

Plate 293T cells (do not carry cells past P+20) at 2×105 cells/well in 0.5 ml DMEM(Dulbecco's Modified Eagle Medium)(with 4.5 GIL glucose and L-glutamine (12-604F Biowhittaker))/10% heat inactivated FBS(14-503F Biowhittaker)/1× Penstrep(17-602EBiowhittaker). Letthe cells grow overnight.

The next day, mix together in a sterile solution basin: 300 ul Lipofectamine (18324-012 Gibco/BRL) and 5 ml Optimem I (31985070 Gibco/BRL)/96-well plate. With a small volume multi-channel pipetter, aliquot approximately 2 ug of an expression vector containing a polynucleotide insert, produced by the methods described in Examples 8-10, into an appropriately labeled 96-well round bottom plate. With a multi-channel pipetter, add 50 ul of the Lipofectamine/Optimem I mixture to each well. Pipette up and down gently to mix. Incubate at RT 15-45 minutes. After about 20 minutes, use a multi-channel pipetter to add 150 ul Optimem I to each well. As a control, one plate of vector DNA lacking an insert should be transfected with each set of transfections.

Preferably, the transfection should be performed by tag-teaming the following tasks. By tag-teaming, hands on time is cut in half, and the cells do not spend too much time on PBS. First, person A aspirates off the media from four 24-well plates of cells, and then person B rinses each well with 0.5-1 ml PBS. Person A then aspirates off PBS rinse, and person B, using a 12-channel pipetter with tips on every other channel, adds the 200 ul of DNA/Lipofectamine/Optimem I complex to the odd wells first, then to the even wells, to each row on the 24-well plates. Incubate at 37° C. for 6 hours.

While cells are incubating, prepare appropriate media, either 1% BSA in DMEM with l× penstrep, or HGS CHO-5 media (116.6 mg/L of CaCI₂ (anhyd); 0.00130 mg/L CuSO₄-5H₂O; 0.050 mg/L of Fe(NO₃)₃-9H₂o; 0.417 mg/L of FeSO₄—7H₂O; 311.80 mg/L of Kcl; 28.64 mg/L of MgCl₂; 48.84 mg/L of MgSO₄; 6995.50 mg/L of NaCl; 2400.0 mg/L of NaHCO₃; 62.50 mg/L of NaH₂PO₄—H₂O; 71.02 mg/L of Na₂HPO₄; 0.4320 mg/L of ZnSO₄—7H₂O; 0.002 mg/L of Arachidonic Acid; 1.022 mg/L of Cholesterol; 0.070 mg/L of DL-alpha-Tocopherol-Acetate; 0.0520 mg/L of Linoleic Acid; 0.010 mg/L of Linolenic Acid; 0.010 mg/L of Myristic Acid; 0.010 mg/L of Oleic Acid; 0.010 mg/L of Palmitric Acid; 0.010 mg/L of Palmitic Acid; 100 mg/L of Pluronic F-68; 0.010 mg/L of Stearic Acid; 2.20 mg/L of Tween 80; 4551 mg/L of D-Glucose; 130.85 mg/ml of L-Alanine; 147.50 mg/ml of L-Arginine-HCL; 7.50 mg/ml of L-Asparagine-H₂O; 6.65 mg/ml of L-Aspartic Acid; 29.56 mg/ml of L-Cystine-2HCL—H₂O; 31.29 mg/ml of L-Cystine-2HCL; 7.35 mg/ml of L-Glutamic Acid; 365.0 mg/ml of L-Glutamine; 18.75 mg/ml of Glycine; 52.48 mg/ml of L-Histidine-HCL-H²O; 106.97 mg/ml of L-Isoleucine; 111.45 mg/ml of L-Leucine; 163.75 mg/ml of L-Lysine HCL; 32.34 mg/ml of L-Methionine; 68.48 mg/ml of L-Phenylalainine; 40.0 mg/ml of L-Proline; 26.25 mg/ml of L-Serine; 101.05 mg/ml of L-Threonine; 19.22 mg/ml of L-Tryptophan; 91.79 mg/ml of L-Tryrosine-2Na-2H₂O; and 99.65 mg/ml of L-Valine; 0.0035 mg/L of Biotin; 3.24 mg/L of D-Ca Pantothenate; 11.78 mg/L of Choline Chloride; 4.65 mg/L of Folic Acid; 15.60 mg/L of i-Inositol; 3.02 mg/L of Niacinamide; 3.00 mg/L of Pyridoxal HCL; 0.031 mg/L of Pyridoxine HCL; 0.319 mg/L of Riboflavin; 3.17 mg/L of Thiamine HCL; 0.365 mg/L of Thymidine; 0.680 mg/L of Vitamin B₁₂; 25 mM of HEPES Buffer; 2.39 mg/L of Na Hypoxanthine; 0.105 mg/L of Lipoic Acid; 0.081 mg/L of Sodium Putrescine-2HCL; 55.0 mg/L of Sodium Pyruvate; 0.0067 mg/L of Sodium Selenite; 20 uM of Ethanolamine; 0.122 mg/L of Ferric Citrate; 41.70 mg/L of Methyl-B-Cyclodextrin complexed with Linoleic Acid; 33.33 mg/L of Methyl-B-Cyclodextrin complexed with Oleic Acid; 10 mg/L of Methyl-B-Cyclodextrin complexed with Retinal Acetate. Adjust osmolarity to 327 mOsm) with 2 mm glutamine and 1× penstrep. (BSA (81-068-3 Bayer) 100 gm dissolved in 1 L DMEM for a 10% BSA stock solution). Filter the media and collect 50 ul for endotoxin assay in 15 ml polystyrene conical.

The transfection reaction is terminated, preferably by tag-teaming, at the end of the incubation period. Person A aspirates off the transfection media, while person B adds 1.5 ml appropriate media to each well. Incubate at 37° C. for 45 or 72 hours depending on the media used: 1% BSA for 45 hours or CHO-5 for 72 hours.

On day four, using a 300 ul multichannel pipetter, aliquot 600 ul in one 1 ml deep well plate and the remaining supernatant into a 2 ml deep well. The supernatants from each well can then be used in the assays described in Examples 14-21.

It is specifically understood that when activity is obtained in any of the assays described below using a supernatant, the activity originates from either the VEGF-3 polypeptide directly (e.g., as a secreted protein) or by VEGF-3 inducing expression of other proteins, which are then secreted into the supernatant. Thus, the invention further provides a method of identifying the protein in the supernatant characterized by an activity in a particular assay.

### Example 13

Construction of GAS Reporter Construct

One signal transduction pathway involved in the differentiation and proliferation of cells is called the Jaks-STATs pathway. Activated proteins in the Jaks-STATs pathway bind to gamma activation site “GAS” elements or interferon-sensitive responsive element (“ISRE”), located in the promoter of many genes. The binding of a protein to these elements alter the expression of the associated gene.

GAS and ISRE elements are recognized by a class of transcription factors called Signal Transducers and Activators of Transcription, or “STATs. ” There are six members of the STATs family. Stat1 and Stat3 are present in many cell types, as is Stat2 (as response to IFN-alpha is widespread). Stat4 is more restricted and is not in many cell types though it has been found in T helper class I, cells after treatment with IL-12. Stat5 was originally called mammary growth factor, but has been found at higher concentrations in other cells including myeloid cells. It can be activated in tissue culture cells by many cytokines.

The STATs are activated to translocate from the cytoplasm to the nucleus upon tyrosine phosphorylation by a set of kinases known as the Janus Kinase (“Jaks”) family. Jaks represent a distinct family of soluble tyrosine kinases and include Tyk2, Jak1, Jak2, and Jak3. These kinases display significant sequence similarity and are generally catalytically inactive in resting cells.

The Jaks are activated by a wide range of receptors summarized in the Table below. (Adapted from review by Schidler and Darnell, Ann. Rev. Biochem. 64:621-51 (1995).) A cytokine receptor family, capable of activating Jaks, is divided into two groups: (a) Class 1 includes receptors for IL-2, IL-3, IL-4, IL-6, IL-7, IL-9, IL-I1, IL-12, IL-15, Epo, PRL, GH, G-CSF, GM-CSF, LIF, CNTF, and thrombopoietin; and (b) Class 2 includes IFN-a, IFN-g, and IL-10. The Class 1 receptors share a conserved cysteine motif (a set of four conserved cysteines and one tryptophan) and a WSXWS motif (a membrane proxial region encoding Trp-Ser-Xxx-Trp-Ser (SEQ ID NO:10)).

Thus, on binding of a ligand to a receptor, Jaks are activated, which in turn activate STATs, which then translocate and bind to GAS elements. This entire process is encompassed in the Jaks-STATs signal transduction pathway.

Therefore, activation of the Jaks-STATs pathway, reflected by the binding of the GAS or the ISRE element, can be used to indicate proteins involved in the proliferation and differentiation of cells. For example, growth factors and cytokines are known to activate the Jaks-STATs pathway. (See Table 1 below.) Thus, by using GAS elements linked to reporter molecules, activators of the Jaks-STATs pathway can be identified.

| | JAKs | | | | | GAS(elements) |
|---|---|---|---|---|---|---|
| Ligand | tyk2 | Jak1 | Jak2 | Jak3 | STATS | or ISRE |
| IFN family | | | | | | |
| IFN-a/B | + | + | − | − | 1, 2, 3 | ISRE |
| IFN-g | | + | + | − | 1 | GAS |
| | | | | | | (IRF1 > |
| | | | | | | Lys6 > IFP) |
| Il-10 | + | ? | ? | − | 1, 3 | |
| gp130 family | | | | | | |
| IL-6 | + | + | + | ? | 1, 3 | GAS |
| (Pleiotrophic) | | | | | | (IRF1 > |
| | | | | | | Lys6 > IFP) |
| Il-11 | ? | + | ? | ? | 1, 3 | |
| (Pleiotrophic) | | | | | | |
| OnM | ? | + | + | ? | 1, 3 | |
| (Pleiotrophic) | | | | | | |
| LIF | ? | + | + | ? | 1, 3 | |
| (Pleiotrophic) | | | | | | |
| CNTF | −/+ | + | + | ? | 1, 3 | |
| (Pleiotrophic) | | | | | | |
| G-CSF | ? | + | ? | ? | 1, 3 | |
| (Pleiotrophic) | | | | | | |
| IL-12 | + | − | + | + | 1, 3 | |
| (Pleiotrophic) | | | | | | |
| g-C family | | | | | | |
| IL-2 | − | + | − | + | 1, 3, 5 | GAS |
| (lymphocytes) | | | | | | |
| IL-4 | − | + | − | + | 6 | GAS |
| (lymph/myeloid) | | | | | | (IRF1 = IFP >> |
| | | | | | | Ly6)(IgH) |
| IL-7 | − | + | − | + | 5 | |
| (lymphocytes) | | | | | | |
| IL-9 | − | + | − | + | 5 | GAS |
| (lymphocytes) | | | | | | |
| IL-13 | − | + | ? | ? | 6 | GAS |
| (lymphocyte) | | | | | | |
| IL-15 | ? | + | ? | + | 5 | GAS |
| gp140 family | | | | | | |
| IL-3 (myeloid) | − | − | + | − | 5 | GAS |
| | | | | | | (IRF1 > |
| | | | | | | IFP °° Ly6) |
| IL-5 (myleoid) | − | − | + | − | 5 | GAS |
| GM-CSF | − | − | + | − | 5 | GAS |
| (myeloid) | | | | | | |
| Growth hormone | | | | | | |
| family | | | | | | |
| GH | ? | − | + | − | 5 | |
| PRL | ? | +/− | + | − | 1, 3, 5 | GAS |
| | | | | | | (B-CAS > |
| | | | | | | IRF1 = |
| | | | | | | IFP >> Ly6) |
| EPO | ? | − | + | − | 5 | |
| Receptor | | | | | | |
| Tyrosine | | | | | | |
| Kinases | | | | | | |
| EGF | ? | + | + | − | 1, 3 | GAS (IRF1) |
| PDGF | ? | + | + | − | 1, 3 | GAS (not |
| | | | | | | IRF1) |
| CSF-1 | ? | + | + | − | 1, 3 | |

To construct a synthetic GAS containing promoter element, which is used in the Biological Assays described in Examples 14-15, a PCR based strategy is employed to generate a GAS-SV40 promoter sequence. The 5′ primer contains four tandem copies of the GAS binding site found in the IRF1 promoter and previously demonstrated to bind STATs upon induction with a range of cytokines (Rothman et al., Immunity 1:457-468 (1994).), although other GAS or ISRE elements can be used instead. The 5′ primer also contains 18 bp of sequence complementary to the SV40 early promoter sequence and is flanked with an XhoI site. The sequence of the 5′ primer is:

| | |
|---|---|
| 5′:GCGCCTCGAGATTTCCCCGAAATCTAGATTTCCCCGAAATGATTTCCCCGAAAT | (SEQ ID NO: 11) |
| GATTTCCCCGAAATATCTGCCATCTCAATTAG:3′ | |

The downstream primer is complementary to the SV40 promoter and is flanked with a Hind III site: 5′:GCGGCAAGCTTTTTGCAAAGCCTAGGC:3′ (SEQ ID NO:12).

PCR amplification is performed using the SV40 promoter template present in the B-gal:promoter plasmid obtained from Clontech. The resulting PCR fragment is digested with XhoI/Hind III and subcloned into BLSK2-. (Stratagene.) Sequencing with forward and reverse primers confirms that the insert contains the following sequence:

| | |
|---|---|
| 5′:CTCGAGATTTCCCCGAAATCTAGATTTCCCCGAAATGATTTCCCCGAAATGATT | (SEQ ID NO: 13) |
| TCCCCGAAATATCTGCCATCTCAATTAGTCAGCAACCATAGTCCCGCCCCTAACT | |
| CCGCCCATCCCGCCCCTAACTCCGCCCAGTTCCGCCCATTCTCCGCCCCATGGCTG | |
| ACTAATTTTTTTTATTTATGCAGAGGCCGAGGCCGCCTCGGCCTCTGAGCTATTCC | |
| AGAAGTAGTGAGGAGGCTTTTTTGGAGGCCTAGGCTTTTGCAAAAAGCTT:3′ | |

With this GAS promoter element linked to the SV40 promoter, a GAS:SEAP2 reporter construct is next engineered. Here, the reporter molecule is a secreted alkaline phosphatase, or “SEAP.” Clearly, however, any reporter molecule can be instead of SEAP, in this or in any of the other Examples. Well known reporter molecules that can be used instead of SEAP include chloramphenicol acetyltransferase (CAT), luciferase, alkaline phosphatase, B-galactosidase, green fluorescent protein (GFP), or any protein detectable by an antibody.

The above sequence confirmed synthetic GAS-SV40 promoter element is subcloned into the p SEAP-Promoter vector obtained from Clontech using HindIII and XhoI, effectively replacing the SV40 promoter with the amplified GAS:SV40 promoter element, to create the GAS-SEAP vector. However, this vector does not contain a neomycin resistance gene, and therefore, is not preferred for mammalian expression systems.

Thus, in order to generate mammalian stable cell lines expressing the GAS-SEAP reporter, the GAS-SEAP cassette is removed from the GAS-SEAP vector using SalI and NotI, and inserted into a backbone vector containing the neomycin resistance gene, such as pGFP-1 (Clontech), using these restriction sites in the multiple cloning site, to create the GAS-SEAP/Neo vector. Once this vector is transfected into mammalian cells, this vector can then be used as a reporter molecule for GAS binding as described in Examples 14-15.

Other constructs can be made using the above description and replacing GAS with a different promoter sequence. For example, construction of reporter molecules containing NFK-B and EGR promoter sequences are described in Examples 16 and 17. However, many other promoters can be substituted using the protocols described in these Examples. For instance, SRE, IL-2, NFAT, or Osteocalcin promoters can be substituted, alone or in combination (e.g., GAS/NF-kB/EGR, GASINF-kB, Il-2/NFAT, or NF-kB/GAS). Similarly, other cell lines can be used to test reporter construct activity, such as HELA (epithelial), HUVEC (endothelial), Reh (B-cell), Saos-2 (osteoblast), HUVAC (aortic), or Cardiomyocyte.

### Example 14

High-throughput Screening Assay for T-cell Activity

The following protocol is used to assess T-cell activity of VEGF-3 by determining whether VEGF-3 supernatant proliferates and/or differentiates T-cells. T-cell activity is assessed using the GAS/SEAP/Neo construct produced in Example 13. Thus, factors that increase SEAP activity indicate the ability to activate the Jaks-STATS signal transduction pathway. The T-cell used in this assay is Jurkat T-cells (ATCC Accession No. TIB-152), although Molt-3 cells (ATCC Accession No. CRL-1552) and Molt-4 cells (ATCC Accession No. CRL-1582) cells can also be used.

Jurkat T-cells are lymphoblastic CD4+ Th1 helper cells. In order to generate stable cell lines, approximately 2 million Jurkat cells are transfected with the GAS-SEAP/neo vector using DMRIE-C (Life Technologies)(transfection procedure described below). The transfected cells are seeded to a density of approximately 20,000 cells per well and transfectants resistant to 1 mg/ml genticin selected. Resistant colonies are expanded and then tested for their response to increasing concentrations of interferon gamma. The dose response of a selected clone is demonstrated.

Specifically, the following protocol will yield sufficient cells for 75 wells containing 200 ul of cells. Thus, it is either scaled up, or performed in multiple to generate sufficient cells for multiple 96 well plates. Jurkat cells are maintained in RPMI +10% serum with 1% Pen-Strep. Combine 2.5 mils of OPTI-MEM (Life Technologies) with 10 ug of plasmid DNA in a T25 flask. Add 2.5 ml OPTI-MEM containing 50 ul of DMRIE-C and incubate at room temperature for 15-45 mins.

During the incubation period, count cell concentration, spin down the required number of cells (10⁷ per transfection), and resuspend in OPTI-MEM to a final concentration of 10⁷ cells/ml. Then add 1 ml of 1×10⁷ cells in OPTI-MEM to T25 flask and incubate at 37° C. for 6 hrs. After the incubation, add 10 ml of RPMI +15% serum.

The Jurkat:GAS-SEAP stable reporter lines are maintained in RPMI+10% serum, 1 mg/ml Genticin, and 1% Pen-Strep. These cells are treated with supernatants containing VEGF-3 polypeptides or VEGF-3 induced polypeptides as produced by the protocol described in Example 12.

On the day of treatment with the supernatant, the cells should be washed and resuspended in fresh RPMI +10% serum to a density of 500,000 cells per ml. The exact number of cells required will depend on the number of supernatants being screened. For one 96 well plate, approximately 10 million cells (for 10 plates, 100 million cells) are required.

Transfer the cells to a triangular reservoir boat, in order to dispense the cells into a 96 well dish, using a 12 channel pipette. Using a 12 channel pipette, transfer 200 ul of cells into each well (therefore adding 100, 000 cells per well).

After all the plates have been seeded, 50 ul of the supernatants are transferred directly from the 96 well plate containing the supernatants into each well using a 12 channel pipette. In addition, a dose of exogenous interferon gamma (0.1, 1.0, 10 ng) is added to wells H9, H10, and H 11 to serve as additional positive controls for the assay.

The 96 well dishes containing Jurkat cells treated with supernatants are placed in an incubator for 48 hrs (note: this time is variable between 48-72 hrs). 35 ul samples from each well are then transferred to an opaque 96 well plate using a 12 channel pipette. The opaque plates should be covered (using sellophene covers) and stored at −20° C. until SEAP assays are performed according to Example 18. The plates containing the remaining treated cells are placed at 4° C. and serve as a source of material for repeating the assay on a specific well if desired.

As a positive control, 100 Unit/ml interferon gamma can be used which is known to activate Jurkat T cells. Over 30 fold induction is typically observed in the positive control wells.

### Example 15

High-throughput Screening Assay Identifying Myeloid Activity

The following protocol is used to assess myeloid activity of VEGF-3 by determining whether VEGF-3 proliferates and/or differentiates myeloid cells. Myeloid cell activity is assessed using the GAS/SEAP/Neo construct produced in Example 13. Thus, factors that increase SEAP activity indicate the ability to activate the Jaks-STATS signal transduction pathway. The myeloid cell used in this assay is U937, a pre-monocyte cell line, although TF-1, HL60, or KG1 can be used.

To transiently transfect U937 cells with the GAS/SEAP/Neo construct produced in Example 13, a DEAE-Dextran method (Kharbanda et. al., Cell Growth & Differentiation 5:259-265 (1994)) is used. First, harvest 2×10e⁷ U937 cells and wash with PBS. The U937 cells are usually grown in RPMI 1640 medium containing 10% heat-inactivated fetal bovine serum (FBS) supplemented with 100 units/ml penicillin and 100 mg/ml streptomycin.

Next, suspend the cells in 1 ml of 20 mM Tris-HCl (pH 7.4) buffer containing 0.5 mg/ml DEAE-Dextran, 8 ug GAS-SEAP2 plasmid DNA, 140 mM NaCl, 5 mM KCl, 375 uM Na2HPO₄.7H₂O, ₁ mM MgCl₂, and 675 uM CaCl₂. Incubate at 37° C. for 45 min.

Wash the cells with RPMI 1640 medium containing 10% FBS and then resuspend in 10 ml complete medium and incubate at 37° C. for 36 hr.

The GAS-SEAP/U937 stable cells are obtained by growing the cells in 400 ug/ml G418. The G418-free medium is used for routine growth but every one to two months, the cells should be re-grown in 400 ug/ml G418 for couple of passages.

These cells are tested by harvesting 1×10⁸ cells (this is enough for ten 96-well plates assay) and wash with PBS. Suspend the cells in 200 ml above described growth medium, with a final density of 5×10⁵ cells/ml. Plate 200 ul cells per well in the 96-well plate (or 1×10⁵ cells/well).

Add 50 ul of the supernatant prepared by the protocol described in Example 12. Incubate at 37° C. for 48 to 72 hr. As a positive control, 100 Unit/ml interferon gamma can be used which is known to activate U937 cells. Over 30 fold induction is typically observed in the positive control wells. SEAP assay the supernatant according to the protocol described in Example 18.

### Example 16

High-throughput Screening Assay Identifying Neuronal Activity

When cells undergo differentiation and proliferation, a group of genes are activated through many different signal transduction pathways. One of these genes, EGRI (early growth response gene 1), is induced in various tissues and cell types upon activation. The promoter of EGRI is responsible for such induction. Using the EGRI promoter linked to reporter molecules, activation of cells can be assessed by VEGF-3.

Particularly, the following protocol is used to assess neuronal activity in PC12 cell lines. PC12 cells (rat phenochromocytoma cells) are known to proliferate and/or differentiate by activation with a number of mitogens, such as TPA (tetradecanoyl phorbol acetate), NGF (nerve growth factor), and EGF (epidermal growth factor). The EGR1 gene expression is activated during this treatment. Thus, by stably transfecting PC12 cells with a construct containing an EGR promoter linked to SEAP reporter, activation of PC12 cells by VEGF-3 can be assessed.

The EGR/SEAP reporter construct can be assembled by the following protocol. The EGR-1 promoter sequence (−633 to +1)(Sakamoto K. et al., Oncogene 6:867-871 (1991)) can be PCR amplified from human genomic DNA using the following primers:

5′ GCGCTCGAGGGATGACAGCGATAGAACCCCGG3′
(SEQ ID NO: 14)
5′ GCGAAGCTTCGCGACTCCCCGGATCCGCCTC-3′
(SEQ ID NO: 15)

Using the GAS:SEAP/Neo vector produced in Example 13, EGR1 amplified product can then be inserted into this vector. Linearize the GAS:SEAP/Neo vector using restriction enzymes XhoI/HindIII, removing the GAS/SV40 stuffer. Restrict the EGRI amplified product with these same enzymes. Ligate the vector and the EGRI promoter.

To prepare 96 well-plates for cell culture, two mls of a coating solution (1:30 dilution of collagen type I (Upstate Biotech Inc. Cat#08-115) in 30% ethanol (filter sterilized)) is added per one 10 cm plate or 50 ml per well of the 96-well plate, and allowed to air dry for 2 hr.

PC12 cells are routinely grown in RPMI-1640 medium (Bio Whittaker) containing 10% horse serum (JRH BIOSCIENCES, Cat. # 12449-78P), 5% heat-inactivated fetal bovine serum (FBS) supplemented with 100 units/ml penicillin and 100 ug/ml streptomycin on a precoated 10 cm tissue culture dish. One to four split is done every three to four days. Cells are removed from the plates by scraping and resuspended with pipetting up and down for more than 15 times.

Transfect the EGR/SEAP/Neo construct into PC12 using the Lipofectamine protocol described in Example 12. EGR-SEAP/PC12 stable cells are obtained by growing the cells in 300 ug/ml G418. The G418-free medium is used for routine growth but every one to two months, the cells should be re-grown in 300 ug/ml G418 for couple of passages.

To assay for neuronal activity, a 10 cm plate with cells around 70 to 80% confluent is screened by removing the old medium. Wash the cells once with PBS (Phosphate buffered saline). Then starve the cells in low serum medium (RPMI-1640 containing 1% horse serum and 0.5% FBS with antibiotics) overnight.

The next morning, remove the medium and wash the cells with PBS. Scrape off the cells from the plate, suspend the cells well in 2 ml low serum medium. Count the cell number and add more low serum medium to reach final cell density as 5×10⁵ cells/ml.

Add 200 ul of the cell suspension to each well of 96-well plate (equivalent to 1×10⁵ cells/well). Add 50 ul supernatant produced by Example 12, 37° C. for 48 to 72 hr. As a positive control, a growth factor known to activate PC12 cells through EGR can be used, such as 50 ng/ul of Neuronal Growth Factor (NGF). Over fifty-fold induction of SEAP is typically seen in the positive control wells. SEAP assay the supernatant according to Example 18.

### Example 17

High-throughput Screening Assay for T-cell Activity

NF-kB (Nuclear Factor kB) is a transcription factor activated by a wide variety of agents including the inflammatory cytokines IL-1 and TNF, CD30 and CD40, lymphotoxin-alpha and lymphotoxin-beta, by exposure to LPS orthrombin, and by expression of certain viral gene products. As a transcription factor, NF-kB regulates the expression of genes involved in immune cell activation, control of apoptosis (NF-Kb appears to shield cells from apoptosis), B and T-cell development, anti-viral and antimicrobial responses, and multiple stress responses.

In non-stimulated conditions, NF-kB is retained in the cytoplasm with I-kB (Inhibitor kB). However, upon stimulation, I-kB is phosphorylated and degraded, causing NF-kB to shuttle to the nucleus, thereby activating transcription of target genes. Target genes activated by NF-kB include IL-2,IL-6, GM-CSF, ICAM-1 and class 1 MHC.

Due to its central role and ability to respond to a range of stimuli, reporter constructs utilizing the NF-kB promoter element are used to screen the supernatants produced in Example 12. Activators or inhibitors of NF-kB would be useful in treating diseases. For example, inhibitors of NF-kB could be used to treat those diseases related to the acute or chronic activation of NF-kB, such as rheumatoid arthritis.

To construct a vector containing the NF-kB promoter element, a PCR based strategy is employed. The upstream primer contains four tandem copies of the NF-kB binding site (GGGGACTTTCCC) (SEQ ID NO:16), 18 bp of sequence complementary to the 5′ end of the SV40 early promoter sequence, and is flanked with an XhoI site:

| | |
|---|---|
| 5′:GCGGCCTCGAGGGGACTTTCCCGGGGACTTTCCGGGGACTTTCCGGGACTTTC | (SEQ ID NO: 17) |
| CATCCTGCCATCTCAATTAG:3′ | |

The downstream primer is complementary to the 3′ end of the SV40promoter and is flanked with a Hind III site: 5′:GCGGCAAGCTTTTTGCAAAGCCTAGGC:3′ (SEQ ID NO:12)

PCR amplification is performed using the SV40 promoter template present in the pB-gal:promoter plasmid obtained from Clontech. The resulting PCR fragment is digested with XhoI and Hind III and subcloned into BLSK2-. (Stratagene) Sequencing with the T7 and T3 primers confirms the insert contains the following sequence:

| | |
|---|---|
| 5′:CTCGAGGGGACTTTCCCGGGGACTTTCCGGGGACTTTCCGGGACTTTCCATCTG | (SEQ ID NO: 18) |
| CCATCTCAATTAGTCAGCAACCATAGTCCCGCCCCTAACTCCGCCCATCCCGCCC | |
| CTAACTCCGCCCAGTTCCGCCCATTCTCCGCCCCATGGCTGACTAATTTTTTTTAT | |
| TTATGCAGAGGCCGAGGCCGCCTCGGCCTCTGAGCTATTCCAGAAGTAGTGAGG | |
| AGGCTTTTTTGGAGGCCTAGGCTTTTGCAAAAAGCTT:3′ | |

Next, replace the SV40 minimal promoter element present in the pSEAP2-promoter plasmid (Clontech) with this NF-kB/SV40 fragment using XhoI and HindIII. However, this vector does not contain a neomycin resistance gene, and therefore, is not preferred for mammalian expression systems.

In order to generate stable mammalian cell lines, the NF-kB/SV40/SEAP cassette is removed from the above NF-kB/SEAP vector using restriction enzymes SalI and NotI, and inserted into a vector containing neomycin resistance. Particularly, the NF-kB/SV40/SEAP cassette was inserted into pGFP-1 (Clontech), replacing the GFP gene, after restricting pGFP-1 with SalI and NotI.

Once NF-kB/SV40/SEAP/Neo vector is created, stable Jurkat T-cells are created and maintained according to the protocol described in Example 14. Similarly, the method for assaying supernatants with these stable Jurkat T-cells is also described in Example 14. As a positive control, exogenous TNF alpha (0.1,1, I 10 ng) is added to wells H9, H10, and H 11, with a 5-10 fold activation typically observed.

### Example 18

Assay for SEAP Activity

As a reporter molecule for the assays described in Examples 14-17, SEAP activity is assayed using the Tropix Phospho-light Kit (Cat. BP-400) according to the following general procedure. The Tropix Phospho-light Kit supplies the Dilution, Assay, and Reaction Buffers used below.

Prime a dispenser with the 2.5× Dilution Buffer and dispense 15 μl of 2.5× dilution buffer into Optiplates containing 35 μl of a supernatant. Seal the plates with a plastic sealer and incubate at 65° C. for 30 min. Separate the Optiplates to avoid uneven heating.

Cool the samples to room temperature for 15 minutes. Empty the dispenser and prime with the Assay Buffer. Add 50 ml Assay Buffer and incubate at room temperature 5 min. Empty the dispenser and prime with the Reaction Buffer (see the table below). Add 50 ml Reaction Buffer and incubate at room temperature for 20 minutes. Since the intensity of the chemiluminescent signal is time dependent, and it takes about 10 minutes to read 5 plates on luminometer, one should treat 5 plates at each time and start the second set 10 minutes later.

Read the relative light unit in the luminometer. Set H12 as blank, and print the results. An increase in chemiluminescence indicates reporter activity.

Reaction Buffer Formulation:

| # of plates | Rxn buffer diluent (ml) | CSPD (ml) |
|---|---|---|
| 10 | 60 | 3 |
| 11 | 65 | 3.25 |
| 12 | 70 | 3.5 |
| 13 | 75 | 3.75 |
| 14 | 80 | 4 |
| 15 | 85 | 4.25 |
| 16 | 90 | 4.5 |
| 17 | 95 | 4.75 |
| 18 | 100 | 5 |
| 19 | 105 | 5.25 |
| 20 | 110 | 5.5 |
| 21 | 115 | 5.75 |
| 22 | 120 | 6 |
| 23 | 125 | 6.25 |
| 24 | 130 | 6.5 |
| 25 | 135 | 6.75 |
| 26 | 140 | 7 |
| 27 | 145 | 7.25 |
| 28 | 150 | 7.5 |
| 29 | 155 | 7.75 |
| 30 | 160 | 8 |
| 31 | 165 | 8.25 |
| 32 | 170 | 8.5 |
| 33 | 175 | 8.75 |
| 34 | 180 | 9 |
| 35 | 185 | 9.25 |
| 36 | 190 | 9.5 |
| 37 | 195 | 9.75 |
| 38 | 200 | 10 |
| 39 | 205 | 10.25 |
| 40 | 210 | 10.5 |
| 41 | 215 | 10.75 |
| 42 | 220 | 11 |
| 43 | 225 | 11.25 |
| 44 | 230 | 11.5 |
| 45 | 235 | 11.75 |
| 46 | 240 | 12 |
| 47 | 245 | 12.25 |
| 48 | 250 | 12.5 |
| 49 | 255 | 12.75 |
| 50 | 260 | 13 |

### Example 19

High-throughput Screening Assay Identifying Changes in Small Molecule Concentration and Membrane Permeability

Binding of a ligand to a receptor is known to alter intracellular levels of small molecules, such as calcium, potassium, sodium, and pH, as well as alter membrane potential. These alterations can be measured in an assay to identify supernatants which bind to receptors of a particular cell. Although the following protocol describes an assay for calcium, this protocol can easily be modified to detect changes in potassium, sodium, pH, membrane potential, or any other small molecule which is detectable by a fluorescent probe.

The following assay uses Fluorometric Imaging Plate Reader (“FLIPR”) to measure changes in fluorescent molecules (Molecular Probes) that bind small molecules. Clearly, any fluorescent molecule detecting a small molecule can be used instead of the calcium fluorescent molecule, fluo-3, used here.

For adherent cells, seed the cells at 10,000-20,000 cells/well in a Co-star black 96-well plate with clear bottom. The plate is incubated in a CO₂ incubator for 20 hours. The adherent cells are washed two times in Biotek washer with 200 ul of HBSS (Hank's Balanced Salt Solution) leaving 100 ul of buffer after the final wash.

A stock solution of 1 mg/ml fluo-3 is made in 10% pluronic acid DMSO. To load the cells with fluo-3, 50 ul of 12 ug/ml fluo-3 is added to each well. The plate is incubated at 37° C. in a C0₂ incubator for 60 min. The plate is washed four times in the Biotek washer with HBSS leaving 100 ul of buffer.

For non-adherent cells, the cells are spun down from culture media. Cells are re-suspended to 2-5×10⁶ cells/ml with HBSS in a 50-ml conical tube. 4 ul of 1 mg/ml fluo-3 solution in 10% pluronic acid DMSO is added to each ml of cell suspension. The tube is then placed in a 37° C. water bath for 30-60 min. The cells are washed twice with HBSS, resuspended to 1×10⁶ cells/ml, and dispensed into a microplate, 100 ul/well. The plate is centrifuged at 1000 rpm for 5 min. The plate is then washed once in Denley CellWash with 200 ul, followed by an aspiration step to 100 ul final volume.

For a non-cell based assay, each well contains a fluorescent molecule, such as fluo-3. The supernatant is added to the well, and a change in fluorescence is detected.

To measure the fluorescence of intracellular calcium, the FLIPR is set for the following parameters: (1) System gain is 300-800 mW; (2) Exposure time is 0.4 second; (3) Camera F/stop is F/2; (4) Excitation is 488 nm; (5) Emission is 530 nm; and (6) Sample addition is 50 ul. Increased emission at 530 nm indicates an extracellular signaling event caused by the a molecule, either VEGF-3 or a molecule induced by VEGF-3, which has resulted in an increase in the intracellular Ca⁺⁺ concentration.

### Example 20

High-throughput Screening Assay Identifying Tyrosine Kinase Activity

The Protein Tyrosine Kinases (PTK) represent a diverse group of transmembrane and cytoplasmic kinases. Within the Receptor Protein Tyrosine Kinase RPTK) group are receptors for a range of mitogenic and metabolic growth factors including the PDGF, FGF, EGF, NGF, HGF and Insulin receptor subfamilies. In addition there are a large family of RPTKs for which the corresponding ligand is unknown. Ligands for RPTKs include mainly secreted small proteins, but also membrane-bound and extracellular matrix proteins.

Activation of RPTK by ligands involves ligand-mediated receptor dimerization, resulting in transphosphorylation of the receptor subunits and activation of the cytoplasmic tyrosine kinases. The cytoplasmic tyrosine kinases include receptor associated tyrosine kinases of the src-family (e.g., src, yes, lck, lyn, fyn) and non-receptor linked and cytosolic protein tyrosine kinases, such as the Jak family, members of which mediate signal transduction triggered by the cytokine superfamily of receptors (e.g., the Interleukins, Interferons, GM-CSF, and Leptin).

Because of the wide range of known factors capable of stimulating tyrosine kinase activity, identifying whether VEGF-3 or a molecule induced by VEGF-3 is capable of activating tyrosine kinase signal transduction pathways is of interest. Therefore, the following protocol is designed to identify such molecules capable of activating the tyrosine kinase signal transduction pathways.

Seed target cells (e.g., primary keratinocytes) at a density of approximately 25,000 cells per well in a 96 well Loprodyne Silent Screen Plates purchased from Nalge Nunc (Naperville, Ill.). The plates are sterilized with two 30 minute rinses with 100% ethanol, rinsed with water and dried overnight. Some plates are coated for 2 hr with 100 ml of cell culture grade type I collagen (50 mg/ml), gelatin (2%) or polylysine (50 mg/ml), all of which can be purchased from Sigma Chemicals (St. Louis, MO) or 10% Matrigel purchased from Becton Dickinson (Bedford, Mass.), or calf serum, rinsed with PBS and stored at 4° C. Cell growth on these plates is assayed by seeding 5,000 cells/well in growth medium and indirect quantitation of cell number through use of alamarBlue as described by the manufacturer Alamar Biosciences, Inc. (Sacramento, Calif.) after 48 hr. Falcon plate covers #3071 from Becton Dickinson (Bedford, Mass.) are used to cover the Loprodyne Silent Screen Plates. Falcon Microtest III cell culture plates can also be used in some proliferation experiments.

To prepare extracts, A431 cells are seeded onto the nylon membranes of Loprodyne plates (20,000/200 ml/well) and cultured overnight in complete medium. Cells are quiesced by incubation in serum-free basal medium for 24 hr. After 5-20 minutes treatment with EGF (60 ng/ml) or 50 ul of the supernatant produced in Example 12, the medium was removed and 100 ml of extraction buffer ((20 mM HEPES pH 7.5, 0.15 M NaCl, 1% Triton X-100, 0.1% SDS, 2 mM Na₃VO₄, 2 mM Na₄P₂O₇ and a cocktail of protease inhibitors (# 1836170) obtained from Boeheringer Mannheim (Indianapolis, Ind.) is added to each well and the plate is shaken on a rotating shaker for 5 minutes at 4° C. The plate is then placed in a vacuum transfer manifold and the extract filtered through the 0.45 mm membrane bottoms of each well using house vacuum. Extracts are collected in a 96-well catch/assay plate in the bottom of the vacuum manifold and immediately placed on ice. To obtain extracts clarified by centrifugation, the content of each well, after detergent solubilization for 5 minutes, is removed and centrifuged for 15 minutes at 4° C. at 16,000 × g.

Test the filtered extracts for levels of tyrosine kinase activity. Although many methods of detecting tyrosine kinase activity are known, one method is described here.

Generally, the tyrosine kinase activity of a supernatant is evaluated by determining its ability to phosphorylate a tyrosine residue on a specific substrate (a biotinylated peptide). Biotinylated peptides that can be used for this purpose include PSK1 (corresponding to amino acids 6-20 of the cell division kinase cdc2-p34) and PSK2 (corresponding to amino acids 1-17 of gastrin). Both peptides are substrates for a range of tyrosine kinases and are available from Boehringer Mannheim.

The tyrosine kinase reaction is set up by adding the following components in order. First, add 10 ul of 5 uM Biotinylated Peptide, then 10 ul ATP/Mg₂₊ (5 mM ATP/50 mM MgC²), then 10 ul of 5× Assay Buffer (40 mM imidazole hydrochloride, pH7.3, 40 mM beta-glycerophosphate, 1 mM EGTA, 100 mM MgCl₂, 5 mM MnCl₂, 0.5 mg/ml BSA), then 5 ul of Sodium Vanadate (1 mM), and then 5 ul of water. Mix the components gently and preincubate the reaction mix at 30° C. for 2 min. Initial the reaction by adding 10 ul of the control enzyme or the filtered supernatant.

The tyro sine kinase assay reaction is then terminated by adding 10 ul of 120 mm EDTA and place the reactions on ice.

Tyrosine kinase activity is determined by transferring 50 ul aliquot of reaction mixture to a microtiter plate (MTP) module and incubating at 37° C. for 20 min. This allows the streptavadin coated 96 well plate to associate with the biotinylated peptide. Wash the MTP module with 300 ul/well of PBS four times. Next add 75 ul of anti-phospotyrosine antibody conjugated to horse radish peroxidase(anti-P-Tyr-POD(0.5 u/ml)) to each well and incubate at 37° C. for one hour. Wash the well as above.

Next add 100 μl of peroxidase substrate solution (Boehringer Mannheim) and incubate at room temperature for at least 5 mins (up to 30 min). Measure the absorbance of the sample at 405 nm by using ELISA reader. The level of bound peroxidase activity is quantitated using an ELISA reader and reflects the level of tyrosine kinase activity.

### Example 21

High-throughput Screening Assay Identifying Phosphorylation Activity

As a potential alternative and/or compliment to the assay of protein tyrosine kinase activity described in Example 20, an assay which detects activation (phosphorylation) of major intracellular signal transduction intermediates can also be used. For example, as described below one particular assay can detect tyrosine phosphorylation of the Erk-1 and Erk-2 kinases. However, phosphorylation of other molecules, such as Raf, JNK, p38 MAP, Map kinase kinase (MEK), MEK kinase, Src, Muscle specific kinase (MuSK), IRAK, Tec, and Janus, as well as any other phosphoserine, phosphotyrosine, or phosphothreonine molecule, can be detected by substituting these molecules for Erk-1 or Erk-2 in the following assay.

Specifically, assay plates are made by coating the wells of a 96-well ELISA plate with 0.1 ml of protein G (1 μg/ml) for 2 hr at room temp, (RT). The plates are then rinsed with PBS and blocked with 3% BSA/PBS for 1 hr at RT. The protein G plates are then treated with 2 commercial monoclonal antibodies (100 ng/well) against Erk-1 and Erk-2 (1 hr at RT) (Santa Cruz Biotechnology). (To detect other molecules, this step can easily be modified by substituting a monoclonal antibody detecting any of the above described molecules.) After 3-5 rinses with PBS, the plates are stored at 4° C. until use.

A 431 cells are seeded at 20,000/well in a 96-well Loprodyne filterplate and cultured overnight in growth medium. The cells are then starved for 48 hr in basal medium (DMEM) and then treated with EGF (6 ng/well) or 50 μl of the supernatants obtained in Example 12 for 5-20 minutes. The cells are then solubilized and extracts filtered directly into the assay plate.

After incubation with the extract for 1 hr at RT, the wells are again rinsed. As a positive control, a commercial preparation of MAP kinase (10 ng/well) is used in place of A431 extract. Plates are then treated with a commercial polyclonal (rabbit) antibody (1 ug/ml) which specifically recognizes the phosphorylated epitope of the Erk-1 and Erk-2 kinases (1 hr at RT). This antibody is biotinylated by standard procedures. The bound polyclonal antibody is then quantitated by successive incubations with Europium-streptavidin and Europium fluorescence enhancing reagent in the Wallac DELFIA instrument (time-resolved fluorescence). An increased fluorescent signal over background indicates a phosphorylation by VEGF-3 or a molecule induced by VEGF-3.

### Example 22

Method of Determining Alterations in the VEGF-3 Gene

RNA isolated from entire families or individual patients presenting with a phenotype of interest (such as a disease) is be isolated. cDNA is then generated from these RNA samples using protocols known in the art. (See, Sambrook.) The cDNA is then used as a template for PCR, employing primers surrounding regions of interest in SEQ ID NO:1. Suggested PCR conditions consist of 35 cycles at 95° C. for 30 seconds; 60-120 seconds at 52-58° C.; and 60-120 seconds at 70° C., using buffer solutions described in Sidransky, D. et al., Science 252:706 (1991).

PCR products are then sequenced using primers labeled at their 5′ end with T4 polynucleotide kinase, employing SequiTherm Polymerase. (Epicentre Technologies). The intron-exon borders of selected exons of VEGF-3 is also determined and genomic PCR products analyzed to confirm the results. PCR products harboring suspected mutations in VEGF-3 is then cloned and sequenced to validate the results of the direct sequencing.

PCR products of VEGF-3 are cloned into T-tailed vectors as described in Holton, T. A. and Graham, M. W., Nucleic Acids Research 19:1156 (1991) and sequenced with T7 polymerase (United States Biochemical). Affected individuals are identified by mutations in VEGF-3 not present in unaffected individuals.

Genomic rearrangements are also observed as a method of determining alterations in the VEGF-3 gene. Genomic clones isolated according to Example 2 are nick-translated with digoxigenindeoxy-uridine 5′ -triphosphate (Boehringer Manheim), and FISH performed as described in Johnson, C g. et al., Methods Cell Biol. 35:73-99 (1991). Hybridization with the labeled probe is carried out using a vast excess of human cot-1 DNA for specific hybridization to the VEGF-3 genomic locus.

Chromosomes are counterstained with 4,6-diamino-2-phenylidole and propidium iodide, producing a combination of C- and R-bands. Aligned images for precise mapping are obtained using a triple-band filter set (Chroma Technology, Brattleboro, Vt.) in combination with a cooled charge-coupled device camera (Photometrics, Tucson, Ariz.) and variable excitation wavelength filters. (Johnson, C v. et al., Genet. Anal. Tech. Appl. 8:75 (1991).) Image collection, analysis and chromosomal fractional length measurements are performed using the ISee Graphical Program System. (Inovision Corporation, Durham, N.C.) Chromosome alterations of the genomic region of VEGF-3 (hybridized by the probe) are identified as insertions, deletions, and translocations. These VEGF-3 alterations are used as a diagnostic marker for an associated disease.

### Example 23

Method ofDetectingAbnormal Levels of VEGF-3 in a Biological Sample

VEGF-3 polypeptides can be detected in a biological sample, and if an increased or decreased level of VEGF-3 is detected, this polypeptide is a marker for a particular phenotype. Methods of detection are numerous, and thus, it is understood that one skilled in the art can modify the following assay to fit their particular needs.

For example, antibody-sandwich ELISAs are used to detect VEGF-3 in a sample, preferably a biological sample. Wells of a microtiter plate are coated with specific antibodies to VEGF-3, at a final concentration of 0.2 to 10 ug/ml. The antibodies are either monoclonal or polyclonal and are produced by the method described in Example 11. The wells are blocked so that non-specific binding of VEGF-3 to the well is reduced.

The coated wells are then incubated for >2 hours at RT with a sample containing VEGF-3. Preferably, serial dilutions of the sample should be used to validate results. The plates are then washed three times with deionized or distilled water to remove unbounded VEGF-3.

Next, 50 ul of specific antibody-alkaline phosphatase conjugate, at a concentration of 25-400 ng, is added and incubated for 2 hours at room temperature. The plates are again washed three times with deionized or distilled water to remove unbounded conjugate.

Add 75 ul of 4-methylumbelliferyl phosphate (MUP) or p-nitrophenyl phosphate (NPP) substrate solution to each well and incubate 1 hour at room temperature. Measure the reaction by a microtiter plate reader. Prepare a standard curve, using serial dilutions of a control sample, and plot VEGF-3 polypeptide concentration on the X-axis (log scale) and fluorescence or absorbance of the Y-axis (linear scale). Interpolate the concentration of the VEGF-3 in the sample using the standard curve.

### Example 24

Formulating a Polypeptide

The VEGF-3 composition will be formulated and dosed in a fashion consistent with good medical practice, taking into account the clinical condition of the individual patient (especially the side effects of treatment with the VEGF-3 polypeptide alone), the site of delivery, the method of administration, the scheduling of administration, and other factors known to practitioners. The “effective amount” for purposes herein is thus determined by such considerations.

As a general proposition, the total pharmaceutically effective amount of VEGF-3 administered parenterally per dose will be in the range of about 1 μg/kg/day to 10 mg/kg/day of patient body weight, although, as noted above, this will be subject to therapeutic discretion. More preferably, this doseis at least 0.01 mg/kg/day, and most preferably for humans between about 0.01 and 1 mg/kg/day for the hormone. If given continuously, VEGF-3 is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing VEGF-3 are administered orally, rectally, parenterally, intracistemally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

VEGF-3 is also suitably administered by sustained-release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2- hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al.) or poly-D-(-)-3-hydroxybutyric acid (EP 133,988). Sustained-release compositions also include liposomally entrapped VEGF-3 polypeptides. Liposomes containing the VEGF-3 are prepared by methods known per se: DE 3,218,121; Epstein et al., Proc. Natl. Acad. Sci. USA 82:3688-3692 (1985); Hwangetal., Proc. Natl. Acad. Sci. USA 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal secreted polypeptide therapy.

For parenteral administration, in one embodiment, VEGF-3 is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting VEGF-3 uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also usefull herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

VEGF-3 is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of polypeptide salts.

VEGF-3 used for therapeutic administration can be sterile. Sterility is readily accomplished by filtration through sterile filtration membranes (e.g., 0.2 micron membranes). Therapeutic polypeptide compositions generally are placed into a container having a sterile access port, for example, an intravenous solution bag or vial having a stopper pierceable by a hypodermic injection needle.

VEGF-3 polypeptides ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous VEGF-3 polypeptide solution, and the resulting mixture is lyophilized. The infusion solutionis prepared by reconstituting thelyophilizedVEGF-3 polypeptide using bacteriostatic Water-for-Injection.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, VEGF-3 may be employed in conjunction with other therapeutic compounds.

### Example 25

Method of Treating Decreased Levels of VEGF-3

The present invention relates to a method for treating an individual in need of a decreased level of VEGF-3 activity in the body comprising, administering to such an individual a composition comprising a therapeutically effective amount of VEGF-3 antagonist. Preferred antagonists for use in the present invention are VEGF-3-specific antibodies.

Moreover, it will be appreciated that conditions caused by a decrease in the standard or normal expression level of VEGF-3 in an individual can be treated by administering VEGF-3, preferably in the secreted form. Thus, the invention also provides a method of treatment of an individual in need of an increased level of VEGF-3 polypeptide comprising administering to such an individual a pharmaceutical composition comprising an amount of VEGF-3 to increase the activity level of VEGF-3 in such an individual.

For example, a patient with decreased levels of VEGF-3 polypeptide receives a daily dose 0.1-100 ug/kg of the polypeptide for six consecutive days. Preferably, the polypeptide is in the secreted form. The exact details of the dosing scheme, based on administration and formulation, are provided in Example 24.

### Example 26

Method of Treating Increased Levels of VEGF-3

The present invention also relates to a method for treating an individual in need of an increased level of VEGF-3 activity in the body comprising administering to such an individual a composition comprising a therapeutically effective amount of VEGF-3 or an agonist thereof

Antisense technology is used to inhibit production of VEGF-3. This technology is one example of a method of decreasing levels of VEGF-3 polypeptide, preferably a secreted form, due to a variety of etiologies, such as cancer.

For example, a patient diagnosed with abnormally increased levels of VEGF-3 is administered intravenously antisense polynucleotides at 0.5, 1.0, 1.5, 2.0 and 3.0 mg/kg day for 21 days. This treatment is repeated after a 7-day rest period if the treatment was well tolerated. The formulation of the antisense polynucleotide is provided in Example 24.

### Example 27

Method of Treatment Using Gene Therapy—Ex Vivo

One method of gene therapy transplants fibroblasts, which are capable of expressing VEGF-3 polypeptides, onto a patient. Generally, fibroblasts are obtained from a subject by skin biopsy. The resulting tissue is placed in tissue-culture medium and separated into small pieces. Small chunks of the tissue are placed on a wet surface of a tissue culture flask, approximately ten pieces are placed in each flask. The flask is turned upside down, closed tight and left at room temperature over night. After 24 hours at room temperature, the flask is inverted and the chunks of tissue remain fixed to the bottom of the flask and fresh media (e.g., Ham's F12 media, with 10% FBS, penicillin and streptomycin) is added. The flasks are then incubated at 37° C. for approximately one week.

At this time, fresh media is added and subsequently changed every several days. After an additional two weeks in culture, a monolayer of fibroblasts emerge. The monolayer is trypsinized and scaled into larger flasks. pMV-7 (Kirschmeier, P. T. et al., DNA 7:219-25 (1988)), flanked by the long terminal repeats of the Moloney murine sarcoma virus, is digested with EcoRi and HindIII and subsequently treated with calfintestinal phosphatase. The linear vector is fractionated on agarose gel and purified, using glass beads.

The cDNA encoding VEGF-3 can be amplified using PCR primers which correspond to the 5′ and 3′ end sequences respectively as set forth in Example 1. Preferably, the 5′ primer contains an EcoRi site and the 3′ primer includes a HindIII site. Equal quantities of the Moloney murine sarcoma virus linear backbone and the amplified EcoRI and HindIII fragment are added together, in the presence of T4 DNA ligase. The resulting mixture is maintained under conditions appropriate for ligation of the two fragments. The ligation mixture is then used to transform bacteria HB101, which are then plated onto agar containing kanamycin for the purpose of confirming that the vector contains properlyinserted VEGF-3.

The amphotropic pA317 or GP+am12 packaging cells are grown in tissue culture to confluent density in Dulbecco's Modified Eagles Medium (DMEM) with 10% calf serum (CS), penicillin and streptomycin. The MSV vector containing the VEGF-3 gene is then added to the media and the packaging cells transduced with the vector. The packaging cells now produce infectious viral particles containing the VEGF-3 gene (the packaging cells are now referred to as producer cells).

Fresh media is added to the transduced producer cells, and subsequently, the media is harvested from a 10 cm plate of confluent producer cells. The spent media, containing the infectious viral particles, is filtered through a millipore filter to remove detached producer cells and this media is then used to infect fibroblast cells. Media is removed from a sub-confluent plate of fibroblasts and quickly replaced with the media from the producer cells. This media is removed and replaced with fresh media. If the titer of virus is high, then virtually all fibroblasts will be infected and no selection is required. If the titer is very low, then it is necessary to use a retroviral vector that has a selectable marker, such as neo or his. Once the fibroblasts have been efficiently infected, the fibroblasts are analyzed to determine whether VEGF-3 protein is produced.

The engineered fibroblasts are then transplanted onto the host, either alone or after having been grown to confluence on cytodex 3 microcarrier beads.

### Example 28

Method of Treatment Using Gene Therapy—In Vivo

Another aspect of the present invention is using in vivo gene therapy methods to treat disorders, diseases and conditions. The gene therapy method relates to the introduction of naked nucleic acid (DNA, RNA, and antisense DNA or RNA) VEGF-3 sequences into an animal to increase or decrease the expression of the VEGF-3 polypeptide. The VEGF-3 polynucleotide may be operatively linked to a promoter or any other genetic elements necessary for the expression of the VEGF-3 polypeptide by the target tissue. Such gene therapy and delivery techniques and methods are known in the art, see, for example, WO90/11092, WO98/11779; U.S. Pat. Nos. 5,693,622, 5,705,151, 5,580,859; Tabata H. et al., Cardiovasc. Res. 35(3):470-479 (1997), Chao J. et al., Pharmacol. Res. 35(6):517-522 (1997), Wolff J. A., Neuromuscul. Disord. 7(5):314-318 (1997), Schwartz B. et al., Gene Ther. 3(5):405-411 (1996), Tsurumi, Y. et al., Circulation 94(12):3281-3290 (1996) (incorporated herein by reference).

The VEGF-3 polynucleotide constructs may be delivered by any method that delivers injectable materials to the cells of an animal, such as, injection into the interstitial space of tissues (heart, muscle, skin, lung, liver, intestine and the like). The VEGF-3 polynucleotide constructs can be delivered in a pharmaceutically acceptable liquid or aqueous carrier.

The term “naked” polynucleotide, DNA or RNA, refers to sequences that are free from any delivery vehicle that acts to assist, promote, or facilitate entry into the cell, including viral sequences, viral particles, liposome formulations, lipofectin or precipitating agents and the like. However, the VEGF-3 polynucleotides may also be delivered in liposome formulations (such as those taught in Felgner, P. L. et al., Ann. NY Acad. Sci. 772:126-139 (1995) and Abdallah, B. et al., Biol. Cell 85(1):1-7 (1995)) which can be prepared by methods well known to those skilled in the art.

The VEGF-3 polynucleotide vector constructs used in the gene therapy method are preferably constructs that will not integrate into the host genome nor will they contain sequences that allow for replication. Any strong promoter known to those skilled in the art can be used for driving the expression of DNA. Unlike other gene therapies techniques, one major advantage ofintroducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The VEGF-3 polynucleotide construct can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked VEGF-3 polynucleotide injection, an effective dosage amount of DNA or RNA will be in the range offrom about 0.05 g/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration. The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked VEGF-3 polynucleotide constructs can be delivered to arteries during angioplasty by the catheter used in the procedure.

The dose response effects of injected VEGF-3 polynucleotide in muscle in vivo is determined as follows. Suitable VEGF-3 template DNA for production of MRNA coding for VEGF-3 polypeptide is prepared in accordance with a standard recombinant DNA methodology. The template DNA, which may be either circular or linear, is either used as naked DNA or complexed with liposomes. The quadriceps muscles of mice are then injected with various amounts of the template DNA.

Five to six week old female and male Balb/C mice are anesthetized by intraperitoneal injection with 0.3 ml of 2.5% Avertin. A 1.5 cm incision is made on the anterior thigh, and the quadriceps muscle is directly visualized. The VEGF-3 template DNA is injected in 0.1 ml of carrier in a 1 cc syringe through a 27 gauge needle over one minute, approximately 0.5 cm from the distal insertion site of the muscle into the knee and about 0.2 cm deep. A suture is placed over the injection site for future localization, and the skin is closed with stainless steel clips.

After an appropriate incubation time (e.g., 7 days) muscle extracts are prepared by excising the entire quadriceps. Every fifth 15 μm cross-section of the individual quadriceps muscles is histochemically stained for VEGF-3 protein expression. A time course for VEGF-3 protein expression may be done in a similar fashion except that quadriceps from different mice are harvested at different times. Persistence of VEGF-3 DNA in muscle following injection may be determined by Southern blot analysis after preparing total cellular DNA and HIRT supernatants from injected and control mice. The results of the above experimentation in mice can be use to extrapolate proper dosages and other treatment parameters in humans and other animals using VEGF-3 naked DNA.

### Example 29

Stimulatory Effect of VEGF-3 on Proliferation of Vascular Endothelial Cells

Experimental Design

VEGF-3 is expressed in highly vascularized tissues, including a high level of expression in colon, and a lower level of expression in heart, kidney, and ovary. The role of VEGF-3 in regulating proliferation of several types of endothelial cells can be examined.

Endothelial Cell Proliferation Assay

For evaluation of mitogenic activity of growth factors, the calorimetric MTS (3-(4,5-dimethylthiazol-2-yl)-5-(3-carboxymethoxyphenyl)-2-(4-sulfophenyl)2H-tetrazolium) assay with the electron coupling reagent PMS (phenazine methosulfate) is performed (CellTiter 96 AQ, Promega). Cells are seeded in a 96-well plate (5,000 cells/well) in 0.1 mL serum-supplemented medium and allowed to attach overnight. After serum-starvation for 12 hours in 0.5% FBS, conditions (bFGF, VEFG₁₆₅ or VEFG-2 in 0.5% FBS) with orwithout Heparin (8 U/ml) are added to wells for 48 hours. 20 μg of MTS/PMS mixture (1:0.05) are added per well and allowed to incubate for 1 hour at 37° C. before measuring the absorbance at 490 nm in an ELISA plate reader. Background absorbance from control wells (some media, no cells) is subtracted, and seven wells are performed in parallel for each condition. See, Leak et al. In Vitro Cell. Dev. Biol 30A:512-518 (1994)

### Example 30

Inhibition of PDGF-induced Vascular Smooth Muscle Cell Proliferation

Smooth muscle is an important therapeutic target for vascular diseases, such as restenosis. To evaluate the potential effects of VEGF-3 on smooth muscle cells, the effect of VEGF-3 on human aortic smooth muscle cell (HAoSMC) proliferation can be examined.

Experimental Design

HAoSMC proliferation can be measured, for example, by BrdUrd incorporation. Briefly, subconfluent, quiescent cells grown on the 4-chamber slides are transfected with CRP or FITC-labeled AT2-3LP. Then, the cells are pulsed with 10% calf serum and 6 μg/ml BrdUrd. After 24 h, immunocytochemistry is performed by using BrdUrd Staining Kit (Zymed Laboratories). In brief, the cells are incubated with the biotinylated mouse anti-BrdUrd antibody at 4° C. for 2 h after exposing to denaturing solution and then with the streptavidin-peroxidase and diaminobenzidine. After counterstaining with hematoxylin, the cells are mounted for microscopic examination, and the BrdUrd-positive cells are counted. The BrdUrd index is calculated as a percent of the BrdUrd-positive cells to the total cell number. In addition, the simultaneous detection of the BrdUrd staining (nucleus) and the FITC uptake (cytoplasm) is performed for individual cells by the concomitant use of bright field illumination and dark field-UV fluorescent illumination. See, Hayashida et al., J. Biol. Chem. 6;271(36):21985-21992 (1996).

### Example 31

Stimulation of Endothelial Cell Migration

Endothelial cell migration is an important step involved in angiogenesis. Experimental Design This example will be used to explore the possibility that VEGF-3 may stimulate lymphatic endothelial cell migration. However, we will be adapting a model of vascular endothelial cell migration for use with lymphatic endothelial cells essentially as follows:

Endothelial cell migration assays are performed using a 48 well microchemotaxis chamber (Neuroprobe Inc., Cabin John, MD; Falk, W., Goodwin, R. H. J., and Leonard, E. J. “A 48 well micro chemotaxis assembly for rapid and accurate measurement of leukocyte migration.” J. Immunological Methods 33:239-247(1980)). Polyvinylpyrrolidone-free polycarbonate filters with a pore size of 8 urn (Nucleopore Corp. Cambridge, Mass.) are coated with 0.1% gelatin for at least 6 hours at room temperature and dried under sterile air. Test substances are diluted to appropriate concentrations in M199 supplemented with 0.25% bovine serum albumin (BSA), and 25 ul of the final dilution is placed in the lower chamber of the modified Boyden apparatus. Subconfluent, early passage (2-6) HUVEC or BMEC cultures are washed and trypsinized for the minimum time required to achieve cell detachment. After placing the filter between lower and upper chamber, 2.5×10⁵ cells suspended in 50 ul M199 containing 1% FBS are seeded in the upper compartment. The apparatus is then incubated for 5 hours at 37° C. in a humidified chamber with 5% CO₂ to allow cell migration. After the incubation period, the filter is removed and the upper side of the filter with the non-migrated cells is scraped with a rubber policeman. The filters are fixed with methanol and stained with a Giemsa solution (Diff-Quick, Baxter, McGraw Park, Ill.). Migration is quantified by counting cells of three random high-power fields (40×) in each well, and all groups are performed in quadruplicate.

### Example 32

Stimulation of Nitric Oxide Production by Endothelial Cells

Nitric oxide released by the vascular endothelium is believed to be a mediator of vascular endothelium relaxation. VEGF-1 has been demonstrated to induce nitric oxide production by endothelial cells in response to VEGF-1. As a result, VEGF-3 activity can be assayed by determining nitric oxide production by endothelial cells in response to VEGF-3.

Experimental Design

Nitric oxide is measured in 96-well plates of confluent microvascular endothelial cells after 24 hours starvation and a subsequent 4 hr exposure to various levels of VEGF-1 and VEGF-3. Nitric oxide in the medium is determined by use of the Griess reagent to measure total nitrite after reduction ofnitric oxide-derived nitrate by nitrate reductase. The effect of VEGF-3 on nitric oxide release can be examined on HUVEC.

Briefly, NO release from cultured HUVEC monolayer is measured with a NO-specific polarographic electrode connected to a NO meter (Iso-NO, World Precision Instruments Inc.) (1049). Calibration of the NO elements is performed according to the following equation:

2KNO₂+2KI+2H₂SO₄→2NO+I₂+2H₂O+2K₂SO₄

The standard calibration curve is obtained by adding graded concentrations of KNO₂ (0, 5, 10, 25, 50, 100, 250, and 500 nmol/L) into the calibration solution containing KI and H₂SO₄. The specificity of the Iso-NO electrode to NO is previously determined bymeasurement ofNO from authenticNO gas (1050). The culture medium is removed and HUVECs are washed twice with Dulbecco's phosphate buffered saline. The cells are then bathed in 5 ml of filtered Krebs-Henseleit solution in 6-well plates, and the cell plates are kept on a slide warmer (Lab Line Instruments Inc.) to maintain the temperature at 37° C. The NO sensor probe is inserted vertically into the wells, keeping the tip of the electrode 2 mm under the surface of the solution, before addition of the different conditions. S-nitroso acetyl penicillamin (SNAP) is used as a positive control. The amount of released NO is expressed as picomoles per 1×10⁶ endothelial cells. See, Leak et al. Biochem. and Biophys. Res. Comm. 217:96-105 (1995).

### Example 33

Effect of VEGF-3 on Cordformation in Angiogenesis

Another step in angiogenesis is cord formation, marked by differentiation of endothelial cells. This bioassay measures the ability of microvascular endothelial cells to form capillary-like structures (hollow structures) when cultured in vitro.

Experimental Design

CADMEC (microvascular endothelial cells) are purchased from Cell Applications, Inc. as proliferating (passage 2) cells and are cultured in Cell Applications' CADMEC Growth Medium and used at passage 5. For the in vitro angiogenesis assay, the wells of a 48-well cell culture plate are coated with Cell Applications' Attachment Factor Medium (200 μl/well) for 30 min. at 37° C. CADMEC are seeded onto the coated wells at 7,500 cells/well and cultured overnight in Growth Medium. The Growth Medium is then replaced with 300 g Cell Applications' Chord Formation Medium containing control buffer or HGS protein (0.1 to 100 ng/ml) and the cells are cultured for an additional 48 hr. The numbers and lengths of the capillary-like chords are quantitated through use of the Boeckeler VIA-170 video image analyzer. All assays are done in triplicate.

Commercial (R&D) VEGF (50 ng/ml) is used as a positive control. β-esteradiol (1 ng/ml) is used as a negative control. The appropriate buffer (without protein) is also utilized as a control.

### Example 34

Angiogenic Effect on Chick Chorioallantoic Membrane

Chick chorioallantoic membrane (CAM) is a well-established system to examine angiogenesis. Blood vessel formation on CAM is easily visible and quantifiable. The ability of VEGF-3 to stimulate angiogenesis in CAM can be examined.

Experimental Design

Embryos

Fertilized eggs of the White Leghorn chick (Gallus gallus) and the Japanese qual (Coturnix coturnix) are incubated at 37.8° C. and 80% humidity. Differentiated CAM of 16-day-old chick and 13-day-old qual embryos are studied with the following methods.

CAM Assay

On Day 4 of development, a window is made into the egg shell of chick eggs. The embryos are checked for normal development and the eggs sealed with cellotape. They are further incubated until Day 13. Thermanox coverslips (Nunc, Naperville, Ill.) are cut into disks of about 5 mm in diameter. Sterile and salt-free growth factors are dissolved in distilled water and about 3.3 μg/5 μl is pipetted on the disks. After air-drying, the inverted disks are applied on CAM. After 3 days, the specimens are fixed in 3% glutaraldehyde and 2% formaldehyde and rinsed in 0.12 M sodium cacodylate buffer. They are photographed with a stereo microscope [Wild M8] and embedded for semi- and ultrathin sectioning as described above. Controls are performed with carrier disks alone.

### Example 35

Angiogenesis Assay using a Matrigel Implant in Mouse

Experimental Design

In order to establish an in vivo model for angiogenesis to test protein activities, mice and rats are implanted subcutaneously with methylcellulose disks containing either 20 mg of BSA (negative control) and 1 mg of bFGF and 0.5 mg of VEFG-1 (positive control).

An additional 30 mice were implanted with disks containing BSA, bFGF, and varying amounts of VEGF-1 and VEGF-3. Each mouse receives two identical disks, rather than one control and one experimental disk.

Samples of all the disks recovered are immunostained with Von Willebrand's factor to detect for the presence of endothelial cells in the disks, and flk-1 and flt-4 to distinguish between vascular and lymphatic endothelial cells.

### Example 36

Rescue of Ischemia in Rabbit Lower Limb Model

Experimental Design

To study the in vivo effects of VEGF-3 on ischemia, a rabbit hindlimb ischemia model is created by surgical removal of one femoral arteries as described previously (Takeshita, S. et al, Am J. Pathol 147:1649-1660 (1995)). The excision of the femoral artery results in retrograde propagation of thrombus and occlusion of the external iliac artery. Consequently, blood flow to the ischemic limb is dependent upon collateral vessels originating from the internal iliac artery (Takeshita, S. et al.,Am J. Pathol 147:1649-1660 (1995)). An interval of 10 days is allowed for post-operative recovery of rabbits and development of endogenous collateral vessels. At 10 day post-operatively (day 0), after performing a baseline angiogram, the internal iliac artery of the ischemic limb is transfected with 500 μg naked VEGF-3 expression plasmid by arterial gene transfer technology using a hydrogel-coated balloon catheter as described (Riessen, R. et al. Hum Gene Ther. 4:749-758 (1993); Leclerc, G. et al. J. Clin. Invest. 90: 936-944 (1992)). When VEGF-3 is used in the treatment, a single bolus of 500 μg VEGF-3 protein or control is delivered into the internal iliac artery of the ischemic limb over a period of 1 min. through an infusion catheter. On day 30, the following parameters are measured in these rabbits.

a. BP Ratio

The blood pressure ratio of systolic pressure of the ischemic limb to that of normal limb.

b. Blood Flow and Flow Reserve

Resting FL: the blood flow during un-dilated condition

Max FL: the blood flow during fully dilated condition (also an indirect measure of the blood vessel amount)

Flow Reserve is reflected by the ratio of max FL: resting FL.

C. Angiographic Score

This is measured by the angiogram of collateral vessels. A score is determined by the percentage of circles in an overlaying grid that with crossing opacified arteries divided by the total number m the rabbit thigh.

d. Capillary density

The number of collateral capillaries determined in light microscopic sections taken from hindlimbs.

### Example 37

Effect of VEGF-3 on Vasodilation

Since dilation of vascular endothelium is important in reducing blood pressure, the ability of VEGF-3 to affect the blood pressure in spontaneously hypertensive rats (SHR) can be examined. Increasing doses (0, 10, 30, 100, 300, and 900 μg/kg) of VEGF-3 are administered to 13-14 week old spontaneously hypertensive rats (SHR). Statistical analysis is performed with a paired t-test and statistical significance is defined as p<0.05 vs. the response to buffer alone. As a control, experiments are performed with another CHO-expressed protein, M-CIF.

### Example 38

Rat Ischemic Skin Flap Model

Experimental Design

The evaluation parameters include skin blood flow, skin temperature, and factor VIII immunohistochemistry or endothelial alkaline phosphatase reaction. VEGF-3 expression, during the skin ischemia, is studied using in situ hybridization.

The study in this model is divided into three parts as follows:

a) Ischemic skin

b) Ischemic skin wounds

c) Normal wounds

The experimental protocol includes:

a) Raising a 3×4 cm, single pedicle full-thickness random skin flap (myocutaneous flap over the lower back of the animal).

b) An excisional wounding (4-6 mm in diameter) in the ischemic skin (skin-flap).

c) Topical treatment with VEGF-3 of the excisional wounds (day 0, 1, 2, 3, 4 post-wounding) at the following various dosage ranges: 1 μg to 100 μg.

d) Harvesting the wound tissues at day 3, 5, 7, 10, 14 and 21 post-wounding for histological, immunohistochemical, and in situ studies.

### Example 39

Peripheral Arterial Disease Model

Angiogenic therapy using VEGF-3 has been developed as a novel therapeutic strategy to obtain restoration of blood flow around the ischemia in case of peripheral arterial diseases.

Experimental Design

The experimental protocol includes:

a) One side of the femoral artery is ligated to create ischemic muscle of the hindlimb, the other side of hindlimb serves as a control.

b) VEGF-3 protein, in a dosage range of 20 μg -500 μg, is delivered intravenously and/or intramuscularly 3 times (perhaps more) per week for 2-3 weeks.

c) The ischemic muscle tissue is collected after ligation of the femoral artery at 1, 2, and 3 weeks for the analysis of VEGF-3 expression and histology.

Biopsy is also performed on the other side of normal muscle of the contralateral hindlimb.

### Example 40

Ischemic Myocardial Disease Model

VEGF-3 is evaluated as a potent mitogen capable of stimulating the development of collateral vessels, and restructuring new vessels after coronary artery occlusion. Alteration of VEGF-3 expression is investigated in situ.

Experimental Design

The experimental protocol includes:

a) The heart is exposed through a left-side thoracotomy in the rat. Immediately, the left coronary artery is occluded with a thin suture (6-0) and the thorax is closed.

b) VEGF-3 protein, in a dosage range of 20 μg -500 μg, is deliveried intravenously and/or intramuscularly 3 times (perhaps more) per week for 2-4 weeks.

c) Thirty days after the surgery, the heart is removed and cross-sectioned for morphometric and in situ analyses.

Example 41

Rat Corneal Wound Healing Model

This animal model shows the effect of VEGF-3 on neovascularization.

Experimental Design

The experimental protocol includes:

a) Making a 1-1.5 mm long incision from the center of cornea into the stromal layer.

b) Inserting a spatula below the lip of the incision facing the outer corner of the eye.

c) Making a pocket (its base is 1-1.5 mm form the edge of the eye).

d) Positioning a pellet, containing 50μg -500μg VEGF-3, within the pocket.

e) VEGF-3 treatment can also be applied topically to the corneal wounds in a dosage range of 20μg -500μg (daily treatment for five days).

### Example 42

Diabetic Mouse and Glucocorticoid-impaired Wound Healing Models

Experimental Design

The experimental protocol includes:

A. Diabetic db+/db+ Mouse Model.

To demonstrate that VEGF-3 accelerates the healing process, the genetically diabetic mouse model of wound healing is used. The full thickness wound healing model in the db+/db+ mouse is a well characterized, clinically relevant and reproducible model of impaired wound healing. Healing of the diabetic wound is dependent on formation of granulation tissue and re-epithelialization rather than contraction (Gartner, M. H. et al., J. Surg. Res. 52:389 (1992); Greenhalgh, D. G. et al., Am. J. Pathol. 136:1235 (1990)).

The diabetic animals have many of the characteristic features observed in Type II diabetes mellitus. Homozygous (db+/db+) mice are obese in comparison to their normal heterozygous (db+/+m) littermates. Mutant diabetic (db+/db+) mice have a single autosomal recessive mutation on chromosome 4 (db+) (Coleman et al. Proc. Natl. Acad. Sci. USA 77:283-293 (1982)). Animals show polyphagia, polydipsia and polyuria. Mutant diabetic mice (db+/db+) have elevated blood glucose, increased or normal insulin levels, and suppressed cell-mediated immunity (Mandel et al., J. Immunol. 120:1375 (1978); Debray-Sachs, M. et al., Clin. Exp. Immunol. 51(1):1-7 (1983); Leiter et al., Am. J. of Pathol. 114:46-55 (1985)). Peripheral neuropathy, myocardial complications, and microvascular lesions, basement membrane thickening and glomerular filtration abnormalities have been described in these animals (Norido, F.et al., Exp. Neurol. 83(2):221-232 (1984); Robertson et al., Diabetes 29(1):60-67 (1980); Giacomelli et al., Lab Invest. 40(4):460-473 (1979); Coleman, D. L., Diabetes 31 (Suppl): 1-6 (1982)). These homozygous diabetic mice develop hyperglycemiathatisresistant to insulin analogous to human type II diabetes (Mandel et al., J. Immunol. 120:1375-1377 (1978)).

The characteristics observed in these animals suggests that healing in this model may be similar to the healing observed in human diabetes (Greenhalgh, et al., Am. J. of Pathol. 136:1235-1246 (1990)).

Animals

Genetically diabetic female C57BL/KsJ (db+/db+) mice and their non-diabetic (db+/+m) heterozygous littermates are used in this study (Jackson Laboratories). The animals are purchased at 6 weeks of age and are 8 weeks old at the beginning of the study. Animals are individually housed and receive food and water ad libitum. All manipulations are performed using aseptic techniques. The experiments are conducted according to the rules and guidelines of Human Genome Sciences, Inc. Institutional Animal Care and Use Committee and the Guidelines for the Care and Use of Laboratory Animals.

Surgical Wounding

Wounding protocol is performed according to previously reported methods (Tsuboi, R. and Rifkin, D. B., J. Exp. Med. 172:245-251 (1990)). Briefly, on the day of wounding, animals are anesthetized with an intraperitoneal injection ofAvertin (0.01 mg/mL), 2,2,2-tribromoethanol and 2-methyl-2-butanol dissolved in deionized water. The dorsal region of the animal is shaved and the skin washed with 70% ethanol solution and iodine. The surgical area is dried with sterile gauze prior to wounding. An 8 mm full-thickness wound is then created using a Keyes tissue punch. Immediately following wounding, the surrounding skin is gently stretched to eliminate wound expansion. The wounds are left open for the duration of the experiment. Application of the treatment is given topically for 5 consecutive days commencing on the day of wounding. Prior to treatment, wounds are gently cleansed with sterile saline and gauze sponges.

Wounds are visually examined and photographed at a fixed distance at the day of surgery and at two day intervals thereafter. Wound closure is determined by daily measurement on days 1-5 and on day 8. Wounds are measured horizontally and vertically using a calibrated Jameson caliper. Wounds are considered healed if granulation tissue is no longer visible and the wound is covered by a continuous epithelium.

VEGF-3 is administered using at a range different doses of VEGF-3, from 4μg to 500 μg per wound per day for 8 days in vehicle. Vehicle control groups receive 50 μL of vehicle solution.

Animals are euthanized on day 8 with an intraperitoneal injection of sodium pentobarbital (300 mg/kg). The wounds and surrounding skin are then harvested for histology and immunohistochemistry. Tissue specimens are placed in 10% neutral buffered formalin in tissue cassettes between biopsy sponges for further processing.

Experimental Design

Three groups of 10 animals each (5 diabetic and 5 non-diabetic controls) are evaluated: 1) Vehicle placebo control, 2) VEGF-3.

Measurement of Wound Area and Closure

Wound closure is analyzed by measuring the area in the vertical and horizontal axis and obtaining the total square area of the wound. Contraction is then estimated by establishing the differences between the initial wound area (day 0) and that of post treatment (day 8). Calculations are made using the following formula:

[Open area on day 8]−[Open area on day 1]/[Open area on day 1]

Histology

Specimens are fixed in 10% buffered formalin and paraffin embedded blocks are sectioned perpendicular to the wound surface (5 μm) and cut using a Reichert-Jung microtome. Routine hematoxylin-eosin (H&E) staining is performed on cross-sections of bisected wounds. Histologic examination of the wounds are used to assess whether the healing process and the morphologic appearance of the repaired skin is altered by treatment with VEGF-3. This assessment included verification of the presence of cell accumulation, inflammatory cells, capillaries, fibroblasts, re-epithelialization and epidermal maturity (Greenhalgh, D. G. et al., Am. J. Pathol. 136:1235 (1990)). A calibrated lens micrometer is used by a blinded observer.

Immunohistochemistry

Re-epithelialization

Tissue sections are stained immunohistochemicallywith apolyclonalrabbit anti-human keratin antibody using ABC Elite detection system. Human skin is used as a positive tissue control while non-immune IgG is used as a negative control. Keratinocyte growth is determined by evaluating the extent of reepithelialization of the wound using a calibrated lens micrometer.

Cell Proliferation Marker

Proliferating cell nuclear antigen/cyclin (PCNA) in skin specimens is demonstrated by using anti-PCNA antibody (1:50) with an ABC Elite detection system. Human colon cancer served as a positive tissue control and human brain tissue is used as a negative tissue control. Each specimen includes a section with omission of the primary antibody and substitution with non-immune mouse IgG. Ranking of these sections is based on the extent of proliferation on a scale of 0-8, the lower side of the scale reflecting slight proliferation to the higher side reflecting intense proliferation.

Statistical Analysis

Experimental data are analyzed using an unpaired t test. A p value of <0.05 is considered significant.

B. Steroid Impaired Rat Model

The inhibition of wound healing by steroids has been well documented in various in vitro and in vivo systems (Wahl, S. M. Glucocorticoids and Wound healing. In Anti-Inflammatory Steroid Action: Basic and Clinical Aspects. 280-302(1989); Wahl, S. M.et al., J. Immunol. 115:476-481 (1975);Werb, Z. et al., J. Exp. Med. 147:1684-1694 (1978)). Glucocorticoids retard wound healing by inhibiting angiogenesis, decreasing vascular permeability (Ebert, R. H., et al., An. Intern. Med. 37:701-705 (1952)), fibroblast proliferation, and collagen synthesis (Beck, L. S. et al., Growth Factors. 5:295-304(1991); Haynes, B. F., et al., J. Clin. Invest. 61: 703-797 (1978)) and producing a transient reduction of circulating monocytes (Haynes, B. F., et al., J. Clin. Invest. 61: 703-797 (1978); Wahl, S. M. Glucocorticoids and wound healing. In Antiinflammatory Steroid Action: Basic and Clinical Aspects. Academic Press. New York. pp. 280-302 (1989)). The systemic administration of steroids to impaired wound healing is a well establish phenomenon in rats (Beck, L. S. et al., Growth Factors. 5: 295-304 (1991); Haynes, B. F., et al., J. Clin. Invest. 61: 703-797 (1978); Wahl, S. M. Glucocorticoids and wound healing. In Antiinflammatory Steroid Action: Basic and Clinical Aspects. Academic Press. New York. pp. 280-302 (1989); Pierce, G. F., et al., Proc. Natl. Acad. Sci. USA. 86: 2229-2233 (1989)).

To demonstrate that VEGF-3 can accelerate the healing process, the effects ofmultiple topical applications of VEGF-3 on full thickness excisional skin wounds in rats in which healing has been impaired by the systemic administration of methylprednisolone is assessed.

Animals

Young adult male Sprague Dawley ratsweighing 250-300 g (Charles River Laboratories) are used in this example. The animals are purchased at 8 weeks of age and were 9 weeks old at the beginning of the study. The healing response of rats is impaired by the systemic administration ofinethylprednisolone (17 mg/kg/rat intramuscularly) at the time of wounding. Animals are individually housed and received food and water ad libitum. All manipulations are performed using aseptic techniques. This study is conducted according to the rules and guidelines of Human Genome Sciences, Inc. Institutional Animal Care and Use Committee and the Guidelines for the Care and Use of Laboratory Animals.

Surgical Wounding

The wounding protocol is followed according to section A, above. On the day of wounding, animals are anesthetized with an intramuscular injection of ketamine (50 mg/kg) and xylazine (5 mg/kg). The dorsal region of the animal is shaved and the skin washed with 70% ethanol and iodine solutions. The surgical area is dried with sterile gauze prior to wounding. An 8 mm full-thickness wound is created using a Keyes tissue punch. The wounds are left open for the duration of the experiment. Applications of the testing materials are given topically once a day for 7 consecutive days commencing on the day ofwounding and subsequent to methylprednisolone administration. Prior to treatment, wounds are gently cleansed with sterile saline and gauze sponges.

Wounds are visually examined and photographed at a fixed distance at the day of wounding and at the end of treatment. Wound closure is determined by daily measurement on days 1-5 and on day 8. Wounds are measured horizontally and vertically using a calibrated Jameson caliper. Wounds are considered healed if granulation tissue was no longer visible and the wound is covered by a continuous epithelium.

VEGF-3 is administered using at a range different doses of VEGF-3, from 4 μg to 500 μg per wound per day for 8 days in vehicle. Vehicle control groups receive 50 μL of vehicle solution.

Animals are euthanized on day 8 with an intraperitoneal injection of sodium pentobarbital (300 mg/kg). The wounds and surrounding skin are then harvested for histology. Tissue specimens are placed in 10% neutral buffered formalin in tissue cassettes between biopsy sponges for further processing.

Experimental Design

Four groups of 10 animals each (5 with methylprednisolone and 5 without glucocorticoid) are evaluated: 1) Untreated group 2) Vehicle placebo control 3) VEGF-3 treated groups.

Measurement of Wound Area and Closure

Wound closure is analyzed by measuring the area in the vertical and horizontal axis and obtaining the total area of the wound. Closure is then estimated by establishing the differences between the initial wound area (day 0) and that of post treatment (day 8). Calculations are made using the following formula:

[Open area on day 8]−[Open area on day 1]/[Open area on day 1]

Histology

Specimens are fixed in 10% buffered formalin and paraffin embedded blocks are sectioned perpendicular to the wound surface (5 μm) and cut using an Olympus microtome. Routine hematoxylin-eosin (H&E) staining is performed on cross-sections of bisected wounds. Histologic examination of the wounds allows assessment of whether the healing process and the morphologic appearance of the repaired skin is improved by treatment with VEGF-3. A calibrated lens micrometer is used by a blinded observer to determine the distance of the wound gap.

Statistical Analysis

Experimental data are analyzed using an unpaired t test. A p value of <0.05 is considered significant.

### Example 42

Lymphadema Animal Model

The purpose of this experimental approach is to create an appropriate and consistent lymphedema model for testing the therapeutic effects of VEGF-3 in lymphangiogenesis and re-establishment of the lymphatic circulatory system in the rat hind limb. Effectiveness is measured by swelling volume of the affected limb, quantification of the amount of lymphatic vasculature, total blood plasma protein, and histopathology. Acute lymphedema is observed for 7-10 days. Perhaps more importantly, the chronic progress of the edema is followed for up to 3-4 weeks.

Experimental Procedure

Prior to beginning surgery, blood sample is drawn for protein concentration analysis. Male rats weighing approximately ˜350 g are dosed with Pentobarbital. Subsequently, the right legs are shaved from knee to hip. The shaved area is swabbed with gauze soaked in 70% EtOH. Blood is drawn for serum total protein testing. Circumference and volumetric measurements are made prior to injecting dye into paws after marking 2 measurement levels (0.5 cm above heel, at mid-pt of dorsal paw). The intradermal dorsum of both right and left paws are injected with 0.05 ml of 1% Evan's Blue. Circumference and volumetric measurements are then made following injection of dye into paws.

Using the knee joint as a landmark, a mid-leg inguinal incision is made circumferentially allowing the femoral vessels to be located. Forceps and hemostats are used to dissect and separate the skin flaps. After locating the femoral vessels, the lymphatic vessel that runs along side and underneath the vessel(s) is located. The main lymphatic vessels in this area are then electrically coagulated or suture ligated.

Using a microscope, muscles in back of the leg (near the semitendinosis and adductors) are bluntly dissected. The popliteal lymph node is then located. The 2 proximal and 2 distal lymphatic vessels and distal blood supply of the popliteal node are then and ligated by suturing. The popliteal lymph node, and any accompanying adipose tissue, is then removed by cutting connective tissues. Care is taken to control any mild bleeding resulting from this procedure. After lymphatics are occluded, the skin flaps are sealed by using liquid skin (Vetbond) (A J Buck). The separated skin edges are sealed to the underlying muscle tissue while leaving a gap of ˜0. 5 cm around the leg. Skin also may be anchored by suturing to underlying muscle when necessary.

To avoid infection, animals are housed individually with mesh (no bedding). Recovering animals are checked daily through the optimal edematous peak, which typically occurs by day 5-7. The plateau edematous peak is then observed. To evaluate the intensity of the lymhedema, the circumference and volumes of 2 designated places on each paw are measured before operation and daily for 7 days. The effect plasma proteins have on lymphedema is determined if protein analysis is a useful testing perimeter is also investigated. The weights of both control and edematous limbs are evaluated at 2 places. Analysis is performed in a blind manner.

Circumference Measurements: Under brief gas anesthetic to prevent limb movement, a cloth tape is used to measure limb circumference. Measurements are done at the ankle bone and dorsal paw by 2 different people then those 2 readings are averaged. Readings are taken from both control and edematous limbs.

Volumetric Measurements: Onthe day of surgery, animals are anesthetized with Pentobarbital and are tested prior to surgery. For daily volumetrics animals are under brief halothane anesthetic (rapid immobilization and quick recovery), both legs are shaved and equally marked using waterproof marker on legs. Legs are first dipped in water, then dipped into instrument to each marked level then measured by Buxco edema software (Chen/Victor). Data is recorded by one person, while the other is dipping the limb to marked area.

Blood-plasma protein measurements: Blood is drawn, spun, and serum separated prior to surgery and then at conclusion for total protein and Ca²⁺ comparison.

Limb Weight Comparison: Afier drawing blood, the animal is prepared for tissue collection. The limbs are amputated using a quillitine, then both experimental and control legs are cut at the ligature and weighed. A second weighing is done as the tibio-cacaneal joint is disarticulated and the foot was weighed.

Histological Preparations: The transverse muscle located behind the knee (popliteal) area is dissected and arranged in a metal mold, filled with freezeGel, dipped into cold methylbutane, placed into labeled sample bags at −80° C. until sectioning. Upon sectioning, the muscle is observed under fluorescent microscopy for lymphatics.

It will be clear that the invention may be practiced otherwise than as particularly described in the foregoing description and examples. Numerous modifications and variations of the present invention are possible in light of the above teachings and, therefore, within the scope of the appended claims, the invention may be practiced otherwise than as particularly described.

The entire disclosure of each document cited (including patents, patent applications, journal articles, abstracts, laboratory manuals, books, or other disclosures) in the Background of the Invention, Detailed Description, Examples, and Sequence Listing is hereby incorporated herein by reference.

What is claimed is:

1. An isolated nucleic acid molecule comprising a polynucleotide having a nucleotide sequence at least 95% identical to a sequence selected from the group consisting of:
(a) a polynucleotide fragment of SEQ ID NO:1 or a polynucleotide fragment of the cDNA sequence included in ATCC Deposit No: 97166;
(b) a polynucleotide encoding a polypeptide fragment of SEQ ID NO:2 or the cDNA sequence included in ATCC Deposit No: 97166;
(c) a polynucleotide encoding a polypeptide domain of SEQ ID NO:2 or the cDNA sequence included in ATCC Deposit No: 97166;
(d) a polynucleotide encoding a polypeptide epitope of SEQ ID NO:2 or the cDNA sequence included in ATCC Deposit No: 97166;
(e) a polynucleotide encoding a polypeptide of SEQ ID NO:2 or the cDNA sequence included in ATCC Deposit No: 97166 having biological activity;
(f) a polynucleotide which is a variant of SEQ ID NO:1
(g) a polynucleotide which is an allelic variant of SEQ ID NO:1;
(h) a polynucleotide which encodes a species homologue of the SEQ ID NO:2;
(i) a polynucleotide capable of hybridizing under stringent conditions to any one of the polynucleotides specified in (a)-(h), wherein said polynucleotide does not hybridize under stringent conditions to a nucleic acid molecule having a nucleotide sequence of only A residues or of only T residues.

2. The isolated nucleic acid molecule of claim 1, wherein the polynucleotide fragment comprises a nucleotide sequence encoding a mature form or a secreted protein.

3. The isolated nucleic acid molecule of claim 1, wherein the polynucleotide fragment comprises a nucleotide sequence encoding the sequence identified as SEQ ID NO:2 orthe coding sequence included in ATCC Deposit No: 97166.

4. The isolated nucleic acid molecule of claim 1, wherein the polynucleotide fragment comprises the entire nucleotide sequence of SEQ ID NO:1 or the cDNA sequence included in ATCC Deposit No: 97166.

5. The isolated nucleic acid molecule of claim 2, wherein the nucleotide sequence comprises sequential nucleotide deletions from either the C-terminus or the N-terminus.

6. The isolated nucleic acid molecule of claim 3, wherein the nucleotide sequence comprises sequential nucleotide deletions from either the C-terminus or the N-terminus.

7. Arecombinant vector comprising the isolated nucleic acid molecule of claim 1.

8. A method of making a recombinant host cell comprising the isolated nucleic acid molecule of claim 1.

9. A recombinant host cell produced by the method of claim 8.

10. The recombinant host cell of claim 9 comprising vector sequences.

11. An isolated polypeptide comprising an amino acid sequence at least 95% identical to a sequence selected from the group consisting of:
(a) a polypeptide fragment of SEQ ID NO:2 or the encoded sequence included in ATCC Deposit No: 97166;
(b) a polypeptide fragment of SEQ ID NO:2 or the encoded sequence included in ATCC Deposit No: 97166 having biological activity;
(c) a polypeptide domain of SEQ ID NO:2 or the encoded sequence included in ATCC Deposit No: 97166;
(d) a polypeptide epitope of SEQ ID NO:2 or the encoded sequence included in ATCC Deposit No: 97166;
(e) a mature form of a secreted protein;
(f) a fall length secreted protein;
(g) a variant of SEQ ID NO:2;
(h) an allelic variant of SEQ ID NO:2; or
(i) a species homologue of the SEQ ID NO:2.

12. The isolated polypeptide of claim 11, wherein the mature form or the full length secreted protein comprises sequential amino acid deletions from either the C-terminus or the N-terminus.

13. An isolated antibody that binds specifically to the isolated polypeptide of claim 11.

14. A recombinant host cell that expresses the isolated polypeptide of claim 11.

15. A method of making an isolated polypeptide comprising:
(a) culturing the recombinant host cell of claim 14 under conditions such that said polypeptide is expressed; and
(b) recovering said polypeptide.

16. The polypeptide produced by claim 15.

17. A method for preventing, treating, or ameliorating a medical condition which comprises administering to a mammalian subject a therapeutically effective amount of the polypeptide of claim 11 or of the polynucleotide of claim 1.

18. Amethod of diagnosing a pathological condition or a susceptibility to a pathological condition in a subject related to expression or activity of a secreted protein comprising:
(a) determining the presence or absence of a mutation in the polynucleotide of claim 1;
(b) diagnosing a pathological condition or a susceptibility to a pathological condition based on the presence or absence of said mutation.

19. Amethod of diagnosing apathological condition or a susceptibility to a pathological condition in a subject related to expression or activity of a secreted protein comprising:
(a) determining the presence or amount of expression of the polypeptide of claim 11 in a biological sample;
(b) diagnosing a pathological condition or a susceptibility to a pathological condition based on the presence or amount of expression of the polypeptide.

20. A method for identifying binding partner to the polypeptide of claim 11 comprising:
(a) contacting the polypeptide of claim 11 with a binding partner; and
(b) determining whether the binding partner effects an activity of the polypeptide.

21. The gene corresponding to the cDNA sequence of SEQ ID NO:2.

22. A method of identifying an activity in a biological assay, wherein the method comprises:
(a) expressing SEQ ID NO:1 in a cell;
(b) isolating the supernatant;
(c) detecting an activity in a biological assay; and
(d) identifying the protein in the supernatant having the activity.

23. The product produced by the method of claim 22.

24. An isolated nucleic acid molecule comprising a polynucleotide having a nucleotide sequence at least 95% identical to a sequence selected from the group consisting of:
(a) a polynucleotide fragment of SEQ ID NO:19;
(b) a polynucleotide encoding a polypeptide fragment of SEQ ID NO:20;
(c) a polynucleotide encoding a polypeptide domain of SEQ ID NO:20;
(d) a polynucleotide encoding a polypeptide epitope of SEQ ID NO:20;
(e) a polynucleotide encoding a polypeptide of SEQ ID NO:20 having biological activity;
(f) a polynucleotide which is a variant of SEQ ID NO:19;
(g) a polynucleotide which is an allelic variant of SEQ ID NO:19;
(h) a polynucleotide which encodes a species homologue of the SEQ ID NO:20;
(i) a polynucleotide capable of hybridizing under stringent conditions to any one of the polynucleotides specified in (a)-(h), wherein said polynucleotide does not hybridize under stringent conditions to a nucleic acid molecule having a nucleotide sequence of only A residues or of only T residues.

25. Anisolatedpolypeptide comprisinganamino acid sequence at least 95% identical to a sequence selected from the group consisting of:
(a) a polypeptide fragment of SEQ ID NO:20;
(b) a polypeptide fragment of SEQ ID NO:20 having biological activity;
(c) a polypeptide domain of SEQ ID NO:20;
(d) a polypeptide epitope of SEQ ID NO:20;
(e) a mature form of a secreted form of SEQ ID No:20;
(f) a full length secreted form of SEQ ID NO:20;
(g) a variant of SEQ ID NO:20;
(h) an allelic variant of SEQ ID NO:20; or
(i) a species homologue of the SEQ ID NO:20.
